(19) United States
(12) Patent Application Publication
(10) Pub. No.: US 2025/0393473 A1
(43) Pub. Date: Dec. 25, 2025

(54) PHOTOELECTRIC CONVERSION ELEMENT, IMAGING ELEMENT, AND OPTICAL SENSOR

(71) Applicant: FUJIFILM CORPORATION, Tokyo (JP)

(72) Inventors: Masaki Morita, Kanagawa (JP); Hiroki Sugiura, Kanagawa (JP); Yasunori Yonekuta, Kanagawa (JP)

(21) Appl. No.: 19/312,469

(22) Filed: Aug. 28, 2025

Related U.S. Application Data
(63) Continuation of application No. PCT/JP2024/009975, filed on Mar. 14, 2024.

(30) Foreign Application Priority Data
Mar. 31, 2023 (JP) 2023-057955

(51) Int. Cl. H10K 85/60; H10K 30/30; H10K 85/20; H10K 85/40

(52) U.S. Cl. CPC H10K 85/6572; H10K 85/211; H10K 85/40; H10K 85/626; H10K 85/654; H10K 85/657; H10K 85/658; H10K 30/30

(57) ABSTRACT

The present invention provides a photoelectric conversion element having excellent responsiveness to green light, and provides an imaging element and an optical sensor each including the photoelectric conversion element. The photoelectric conversion element of the present invention is a photoelectric conversion element including a conductive film, a photoelectric conversion film, and a transparent conductive film in this order, in which the photoelectric conversion film contains a first compound represented by Formula (1) and a second compound which is a compound different from the first compound, and a maximal absorption wavelength λ1 of the first compound and a maximal absorption wavelength λ2 of the second compound satisfy a relationship of an expression (X).

$$- 20 nm \leq λ 1 - λ 2 \leq 20 nm \qquad Expression ( X )$$

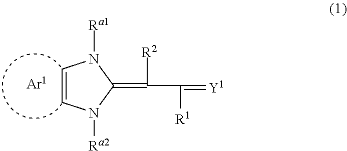

### CROSS-REFERENCE TO RELATED APPLICATIONS

This application is a Continuation of PCT International Application No. PCT/JP2024/009975 filed on Mar. 14, 2024, which claims priority under 35 U.S.C. § 119(a) to Japanese Patent Application No. 2023-057955 filed on Mar. 31, 2023. The above applications are hereby expressly incorporated by reference, in their entirety, into the present application.

### BACKGROUND OF THE INVENTION

### 1. Field of the Invention

The present invention relates to a photoelectric conversion element, an imaging element, and an optical sensor.

### 2. Description of the Related Art

In recent years, development of an element (for example, an imaging element) having a photoelectric conversion film has progressed.

For example, WO2021/141078A discloses, as a photoelectric conversion element having excellent suppression properties of a change in external quantum efficiency during continuous driving and excellent suppression properties of a change in dark current during continuous driving in a case of being applied to panchromatic light, “photoelectric conversion element including a conductive film, a photoelectric conversion film, and a transparent conductive film in this order, in which the photoelectric conversion film contains a first compound which has a maximal absorption wavelength in a wavelength of 500 to 620 nm, does not have an ionic group, and is represented by Formula (1), and a second compound which is different from the first compound and has a maximal absorption wavelength in a wavelength of 450 to 550 nm”.

### SUMMARY OF THE INVENTION

In a case where the present inventors have evaluated responsiveness (response speed) to green light (wavelength of 490 to 600 nm) using the photoelectric conversion element having the configuration disclosed in WO2021/141078A, it is found that the responsiveness does not satisfy the higher requirement level in recent years and further improvement is required.

Therefore, an object of the present invention is to provide a photoelectric conversion element having excellent responsiveness to green light.

Another object of the present invention is to provide an imaging element and an optical sensor, including the above-described photoelectric conversion element.

As a result of conducting an extensive investigation to achieve the objects, the present inventors have found that the objects can be achieved by the following constitution.
[1]A photoelectric conversion element comprising, in the following order:
a conductive film;
a photoelectric conversion film; and
a transparent conductive film,
in which the photoelectric conversion film contains a first compound represented by Formula (1) described later and a second compound which is a compound different from the first compound, and
a maximal absorption wavelength λ1 of the first compound and a maximal absorption wavelength λ2 of the second compound satisfy a relationship of an expression (X),

$$- 20 nm \leq λ 1 - λ 2 \leq 20 nm . \qquad Expression ( X )$$

[2] The photoelectric conversion element according to [1],
in which the first compound is a compound represented by Formula (2) described later.
[3] The photoelectric conversion element according to [2],
in which X¹ and X⁴ in Formula (2) represent a nitrogen atom.
[4] The photoelectric conversion element according to any one of [1] to [3],
in which the first compound is a compound represented by Formula (3) described later.
[5] The photoelectric conversion element according to any one of [1] to [4],
in which the second compound is a compound selected from the group consisting of a compound having an imidazoline skeleton, a pyrromethene boron complex, a subphthalocyanine compound, a squarylium compound, and a compound having a triarylamine skeleton.
[6] The photoelectric conversion element according to any one of [1] to [5],
in which the second compound is a compound represented by Formula (11) described later.
[7] The photoelectric conversion element according to any one of [1] to [6],
in which the photoelectric conversion film further contains an n-type semiconductor material, and
the photoelectric conversion film has a bulk heterojunction structure formed in a state in which the first compound, the second compound, and the n-type semiconductor material are mixed with each other.
[8] The photoelectric conversion element according to [7],
in which the n-type semiconductor material includes fullerenes selected from the group consisting of a fullerene and derivatives of the fullerene.
[9] The photoelectric conversion element according to any one of [1] to [8],
in which the photoelectric conversion film further contains an n-type semiconductor material and a p-type semiconductor material, and
the photoelectric conversion film has a bulk heterojunction structure formed in a state in which the first compound, the second compound, the n-type semiconductor material, and the p-type semiconductor material are mixed with each other.
[10] The photoelectric conversion element according to [9],
in which the n-type semiconductor material includes fullerenes selected from the group consisting of a fullerene and derivatives of the fullerene.
[11] The photoelectric conversion element according to [9] or [10],
in which the p-type semiconductor material has no absorption in a visible light region.
[12] The photoelectric conversion element according to any one of [1] to [11], further comprising:
one or more interlayers between the conductive film and the transparent conductive film, in addition to the photoelectric conversion film.
[13] An imaging element comprising:
the photoelectric conversion element according to any one of [1] to [12].
[14] An optical sensor comprising:
the photoelectric conversion element according to any one of [1] to [12].

According to the present invention, it is possible to provide a photoelectric conversion element having excellent responsiveness to green light.

In addition, it is possible to provide an imaging element and an optical sensor, including the above-described photoelectric conversion element.

### BRIEF DESCRIPTION OF THE DRAWINGS

FIG. 1 is a schematic cross-sectional view showing an example of a configuration of a photoelectric conversion element.

FIG. 2 is a schematic cross-sectional view showing an example of a configuration of another photoelectric conversion element.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, the “substituent” includes a group exemplified as the following substituent W, unless otherwise specified.

### (Substituent W)

Examples of the substituent W include a halogen atom (a fluorine atom, a chlorine atom, a bromine atom, an iodine atom, or the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (may be referred to as a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, and a silyl group. In addition, each of the above-described groups may further have a substituent (for example, one or more groups of each of the above-described groups), as possible. For example, an alkyl group which may have a substituent is also included as the form of the substituent W.

In addition, in a case where the substituent W has a carbon atom, the number of carbon atoms in the substituent W is, for example, 1 to 20.

The number of atoms other than a hydrogen atom in the substituent W is, for example, 1 to 30.

In addition, from the viewpoint of appropriately adjusting vapor deposition suitability, it is also preferable that the first compound, the second compound, the n-type semiconductor material, and/or the p-type semiconductor material, which will be described later, do not have, as a substituent, a carboxy group, a salt of a carboxy group, a phosphoric acid group, a salt of a phosphoric acid group, a sulfonic acid group, a salt of a sulfonic acid group, a hydroxy group, an SH group, an acylamino group, a carbamoyl group, a ureido group, a boronic acid group (—B(OH)₂), and/or —NH₂.

In the present specification, examples of the halogen atom include a fluorine atom, a chlorine atom, a bromine atom, and an iodine atom.

In addition, in the present specification, unless otherwise specified, the number of carbon atoms in the alkyl group is preferably 1 to 20, more preferably 1 to 10, and still more preferably 1 to 6.

The alkyl group may be linear, branched, or cyclic.

Examples of the alkyl group include a methyl group, an ethyl group, a n-propyl group, an i-propyl group, a n-butyl group, a tert-butyl group, a n-hexyl group, and a cyclopentyl group.

In addition, the alkyl group may be a cycloalkyl group, a bicycloalkyl group, or a tricycloalkyl group, and may have a cyclic structure thereof as a partial structure.

In the alkyl group which may have a substituent, a substituent which may be included in the alkyl group is not particularly limited, an example thereof includes the substituent W; and an aryl group (preferably having 6 to 18 carbon atoms and more preferably having 6 carbon atoms), a heteroaryl group (preferably having 5 to 18 carbon atoms and more preferably having 5 and 6 carbon atoms), or a halogen atom (preferably a fluorine atom or a chlorine atom) is preferable.

In addition, examples of the alkylene group in the present specification include an alkylene group in which one hydrogen atom is removed from the above-described alkyl group to form a divalent group.

In the present specification, unless otherwise specified, an alkyl group moiety in the alkoxy group is preferably the above-described alkyl group. An alkyl group moiety in the alkylthio group is preferably the above-described alkyl group.

In the alkoxy group which may have a substituent, examples of the substituent which may be included in the alkoxy group include the same examples as the substituent in the alkyl group which may have a substituent. In the alkylthio group which may have a substituent, examples of the substituent which may be included in the alkylthio group include the same examples as the substituent in the alkyl group which may have a substituent.

In the present specification, the alkenyl group may be any of linear, branched, or cyclic, unless otherwise specified. The number of carbon atoms in the above-described alkenyl group is preferably 2 to 20. In the alkenyl group which may have a substituent, examples of the substituent which may be included in the alkenyl group include the same examples as the substituent in the alkyl group which may have a substituent.

In addition, examples of the alkenylene group in the present specification include an alkenylene group obtained by removing one hydrogen atom from the above-described alkenyl group to form a divalent group.

In the present specification, the alkynyl group may be any of linear, branched, or cyclic, unless otherwise specified. The number of carbon atoms in the above-described alkynyl group is preferably 2 to 20. In the alkynyl group which may have a substituent, examples of the substituent which may be included in the alkynyl group include the same examples as the substituent in the alkyl group which may have a substituent.

In addition, examples of the alkynylene group in the present specification include an alkynylene group obtained by removing one hydrogen atom from the above-described alkynyl group to form a divalent group.

In the present specification, unless otherwise specified, the aryl group is preferably an aryl group having 6 to 18 ring members.

The aryl group may be a monocyclic ring or a polycyclic ring (for example, 2 to 6 rings).

The aryl group is preferably, for example, a phenyl group, a naphthyl group, an anthryl group, or a phenanthrenyl group.

In the aryl group which may have a substituent, the substituent which may be included in the aryl group is not particularly limited, and an example thereof includes the substituent W; and an alkyl group (preferably having 1 to 10 carbon atoms) which may have a substituent is preferable, and a methyl group is more preferable.

In a case where the aryl group which may have a substituent has a plurality of substituents, the plurality of substituents may be bonded to each other to form a ring. In a case where a plurality of substituents are bonded to each other to form a ring, for example, the aryl group which may have a substituent may further form, as a whole, a fluorenyl group (9,9-dimethylfluorenyl group or the like) which may further have a substituent.

In addition, examples of the arylene group in the present specification include an arylene group obtained by removing one hydrogen atom from the ring member atom of the above-described aryl group to form a divalent group.

In the present specification, unless otherwise specified, the heteroaryl group is preferably a heteroaryl group having a monocyclic or polycyclic ring structure, which contains a heteroatom such as a nitrogen atom, a sulfur atom, an oxygen atom, a selenium atom, a tellurium atom, a phosphorus atom, a silicon atom, and/or a boron atom.

The number of carbon atoms in the ring member atoms of the above-described heteroaryl group is not particularly limited, but is preferably 3 to 18 and more preferably 3 to 5.

The number of heteroatoms in the ring member atoms of the heteroaryl group is not particularly limited, but is preferably 1 to 10 more preferably 1 to 4 and still more preferably 1 or 2.

The heteroaryl group may be a monocyclic ring or a polycyclic ring (for example, 2 to 6 rings).

The number of ring members in the heteroaryl group is not particularly limited, but is preferably 5 to 15.

Examples of the heteroaryl group include a furyl group, a pyridyl group, a quinolyl group, an isoquinolyl group, an acridinyl group, a phenanthridinyl group, a pteridinyl group, a pyrazinyl group, a quinoxalinyl group, a pyrimidinyl group, a quinazolyl group, a pyridazinyl group, a cinnolinyl group, a phthalazinyl group, a triazinyl group, an oxazolyl group, a benzoxazolyl group, a thiazolyl group, a benzothiazolyl group, an imidazolyl group, a benzimidazolyl group, a pyrazolyl group, an indazolyl group, an isoxazolyl group, a benzisoxazolyl group, an isothiazolyl group, a benzisothiazolyl group, an oxadiazolyl group, a thiadiazolyl group, a triazolyl group, a tetrazolyl group, a benzofuryl group, a thienyl group, a benzothienyl group, a dibenzofuryl group, a dibenzothienyl group, a pyrrolyl group, an indolyl group, an imidazopyridinyl group, and a carbazolyl group.

In the heteroaryl group which may have a substituent, the substituent which may be included in the heteroaryl group is not particularly limited, and an example thereof includes the substituent W.

In a case where the heteroaryl group which may have a substituent has a plurality of substituents, the plurality of substituents may be bonded to each other to form a ring.

In addition, examples of the heteroarylene group in the present specification include a heteroarylene group obtained by removing one hydrogen atom from the ring member atom of the above-described heteroaryl group to form a divalent group.

In the present specification, unless otherwise specified, the concept of an aromatic ring includes both an aromatic hydrocarbon ring and an aromatic heterocyclic ring.

In a case where the above-described aromatic ring group is monovalent, examples of the aromatic ring group include the above-described aryl group and heteroaryl group.

In a case where the above-described aromatic ring group is m-valent (m is an integer of 2 or more, preferably 2 to 5), examples of the aromatic ring group include a group obtained by removing (m−1) hydrogen atoms from the ring member atom of the above-described aryl group or heteroaryl group.

In the present specification, unless otherwise specified, examples of the silyl group which may have a substituent include a group represented by —Si(R^{S1})(R^{S2})(R^{S3}). R^{S1}, R^{S2}, and R^{S3} each independently represent a hydrogen atom or a substituent, and preferably represent an alkyl group which may have a substituent, an alkoxy group which may have a substituent, an alkylthio group which may have a substituent, an aryl group which may have a substituent, or a heteroaryl group which may have a substituent.

A bonding direction of a divalent group (for example, —CO—O—) denoted in the present specification is not limited unless otherwise specified. For example, in a case where Y in a compound represented by a general formula “X—Y—Z” is —CO—O—, the compound may be “X—O—CO—Z” or “X—CO—O—Z”.

A compound described in the present specification may include a structural isomer, an optical isomer, and an isotope unless otherwise specified. In addition, one kind of structural isomer, optical isomer, and isotope may be included, or two or more kinds thereof may be included.

In the present specification, regarding a compound which may have a geometric isomer (cis-trans isomer), a general formula or a structural formula representing the compound may be described only in the form of either a cis isomer or a trans isomer for convenience. Even in such a case, unless otherwise specified, the form of the compound is not limited to either the cis isomer or the trans isomer, and the compound may be the cis isomer or the trans isomer.

In addition, in the present specification, numerical ranges represented by “to” include numerical values before and after “to” as lower limit values and upper limit values.

In the present specification, a hydrogen atom may be a light hydrogen atom (normal hydrogen atom) or a heavy hydrogen atom (a deuterium atom or the like).

In the present specification, a maximal absorption wavelength of each compound (the first compound, the second compound, and the like) can be calculated from an absorption spectrum measured using a solution obtained by dissolving the compound in chloroform. A concentration of the compound in the above-described solution is adjusted to a concentration such that an absorbance at the maximal absorption wavelength is 0.5 to 1. In addition, in a case where the above-described maximal absorption wavelength is located in a visible light region (wavelength of 400 to 700 nm) and a plurality of maximal absorption wavelengths are observed, the wavelength having the highest absorbance is defined as the maximal absorption wavelength. However, in a case where the compound is not soluble in chloroform, a value measured by using the compound vapor-deposited and formed into a film is defined as the maximal absorption wavelength of the compound.

### [Photoelectric Conversion Element]

Hereinafter, the photoelectric conversion element according to the embodiment of the present invention will be described in detail.

The photoelectric conversion element according to the embodiment of the present invention is a photoelectric conversion element including a conductive film, a photoelectric conversion film, and a transparent conductive film in this order, in which the photoelectric conversion film contains a first compound represented by Formula (1) and a second compound which is different from the first compound, and a maximal absorption wavelength λ1 of the first compound and a maximal absorption wavelength λ2 of the second compound satisfy a relationship of an expression (X) described later.

The mechanism by which the photoelectric conversion element according to the embodiment of the present invention can achieve the above-described problems is not necessarily clear, but the present inventors assume as follows.

The mechanism by which the effect is obtained is not limited by the following supposition. In other words, even in a case where an effect is obtained by a mechanism other than the following, it is included in the scope of the present invention.

The photoelectric conversion element disclosed in WO2021/141078A contains a first compound having a maximal absorption wavelength in a wavelength of 500 to 620 nm and a second compound having a maximal absorption wavelength in a wavelength of 450 to 550 nm, thereby improving element performance (for example, external quantum efficiency) with respect to light in a wide visible light region (panchromatic range). Therefore, although the performance for the panchromatic use application is excellent, it has been difficult to obtain a high level of performance required in recent years for element performance (particularly, responsiveness) with respect to green light, which is an object of the present invention.

In the photoelectric conversion element according to the embodiment of the present invention, the element performance for green light is improved by mixing two kinds of coloring agents having maximal absorption wavelengths close to each other. In general, in a case where two or more coloring agents are mixed, a shape of an absorption spectrum is a broad spectrum obtained by averaging the spectra of the respective coloring agents, and generally, it is difficult to obtain a sharp spectrum. In addition, in many cases, it is difficult to obtain selective element performance for green light only by mixing the two kinds of coloring agents.

On the other hand, surprisingly, in the embodiment of the present invention, it has been confirmed that absorption of the photoelectric conversion element with respect to blue light is reduced by mixing the first compound and the second compound having a maximal absorption wavelength close to that of the first compound, as specific coloring agents. Although the detailed mechanism is not clear, it is considered that the first compound has an asymmetric structure, so that aggregation of coloring agents can be suppressed, and thus the absorption of blue light can be suppressed and the element performance (for example, responsiveness to green light) can be improved selectively for green light.

Hereinafter, the fact that the responsiveness to green light (in particular, responsiveness to light having a wavelength of 560 nm) is more excellent is also simply referred to as “effect of the present invention is more excellent”.

In addition, hereinafter, the first compound and the second compound are also collectively referred to as a specific compound.

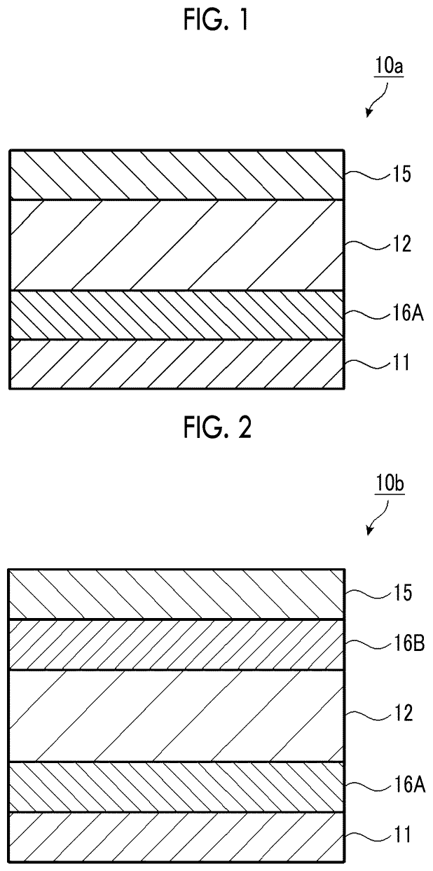

FIG. 1 shows a schematic cross-sectional view of one embodiment of the photoelectric conversion element according to the embodiment of the present invention.

A photoelectric conversion element 10a shown in FIG. 1 has a configuration in which a conductive film (hereinafter, also referred to as a lower electrode) 11 functioning as a lower electrode, an electron blocking film 16A, a photoelectric conversion film 12, and a transparent conductive film (hereinafter, also referred to as an upper electrode) 15 functioning as an upper electrode are laminated in this order.

The above-described photoelectric conversion film 12 contains the above-described first compound and the above-described second compound.

The above-described photoelectric conversion film 12 may be a monolayer type consisting of one layer, or a laminated type consisting of a plurality of layers.

In a case where the above-described photoelectric conversion film 12 is a monolayer type, for example, the above-described photoelectric conversion film 12 may be a mixed layer formed in a state in which the first compound and the second compound are mixed.

FIG. 2 shows a configuration example of another photoelectric conversion element. A photoelectric conversion element 10b shown in FIG. 2 has a configuration in which the electron blocking film 16A, the photoelectric conversion film 12, a hole blocking film 16B, and the upper electrode 15 are laminated on the lower electrode 11 in this order. The lamination order of the electron blocking film 16A, the photoelectric conversion film 12, and the hole blocking film 16B in FIGS. 1 and 2 may be appropriately changed according to the application and the characteristics.

The photoelectric conversion film 12 in FIG. 2 may be a monolayer type photoelectric conversion film 12 consisting of one layer, or a laminated type photoelectric conversion film 12 consisting of a plurality of layers.

In the photoelectric conversion element 10a (or 10b), it is preferable that light is incident to the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of performance and power consumption, the applied voltage is more preferably 1×10⁴ to 1×10⁷ V/cm and still more preferably 1×10⁻³ to 5×10⁶ V/cm.

Regarding the voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case of being incorporated in an imaging element, the voltage can be applied by the same method.

As described in detail below, the photoelectric conversion element 10a (or 10b) can be suitably applied to applications of an imaging element.

Hereinafter, the form of each layer constituting the photoelectric conversion element according to the embodiment of the present invention will be described in detail.

Hereinafter, the form of each layer constituting the photoelectric conversion element according to the embodiment of the present invention will be described in detail.

### <Photoelectric Conversion Film>

As described above, the photoelectric conversion film is a film containing the first compound and the second compound.

### (First Compound)

The first compound will be described.

The first compound is a compound represented by Formula (1), and a maximal absorption wavelength λ1 of the first compound and a maximal absorption wavelength λ2 of a second compound, which will be described later, satisfy a relationship of an expression (X) described later.

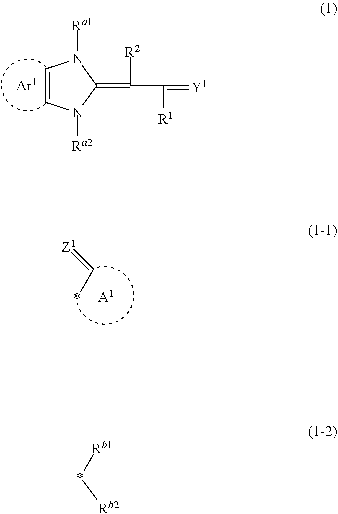

In Formula (1), Y¹ represents a group represented by Formula (1-1) or a group represented by Formula (1-2). Among these, from the viewpoint that the effect of the present invention is more excellent, a group represented by Formula (1-1) is preferable. In Formulae (1-1) and (1-2), * represents a bonding position, and a carbon atom marked with * and a carbon atom bonded to R¹ form a double bond.

That is, the compound represented by Formula (1) is a compound represented by Formula (1-1a) or a compound represented by Formula (1-2a).

The symbols used in Formulae (1-1a) and (1-2a) have the same meanings as the corresponding symbols used in Formula (1).

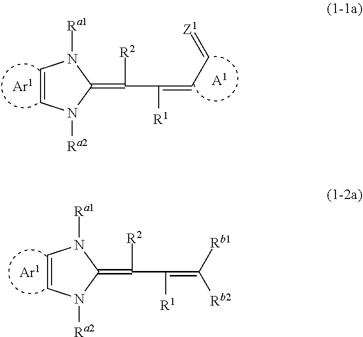

In Formula (1-1), Z¹ represents an oxygen atom, a sulfur atom, ═NR^{Z1}, or ═CR^{Z2}R^{Z3}.

R^{Z1} represents a hydrogen atom or a substituent. R^{Z2} and R^{Z3} each independently represent a cyano group or —COOR^{Z4}. R^{Z4} represents an alkyl group which may have a substituent, an aryl group which may have a substituent, or a heteroaryl group which may have a substituent.

Z¹ is preferably an oxygen atom.

In Formula (1-1), A¹ represents a ring which contains at least two carbon atoms and may have a substituent. The two carbon atoms mean a carbon atom which is bonded to Z¹ specified in Formula (1-1) and a carbon atom which is adjacent to the carbon atom bonded to Z¹ and is specified in Formula (1-1) (a carbon atom forming the double bond with the carbon atom bonded to R¹), and any of the two carbon atoms is an atom constituting A¹.

In addition, in the above-described ring, carbon atoms constituting the ring may be substituted with a carbonyl carbon (>C═O) and/or a thiocarbonyl carbon (>C═S). The carbonyl carbon (>C═O) and the thiocarbonyl carbon (>C═S) as used herein mean a carbonyl carbon and a thiocarbonyl carbon, each of which has, as a constituent, a carbon atom other than the carbon atom bonded to Z¹ among the carbon atoms constituting the ring.

The number of carbon atoms in A¹ is preferably 3 to 30, more preferably 3 to 20, and still more preferably 3 to 15. The above-described number of carbon atoms is a number including two carbon atoms specified in the formula.

A¹ may have a heteroatom, and examples thereof include a nitrogen atom, a sulfur atom, an oxygen atom, a selenium atom, a tellurium atom, a phosphorus atom, a silicon atom, and a boron atom.

The number of heteroatoms in A¹ is preferably 0 to 10, more preferably 0 to 5, and still more preferably 0 to 2. The number of heteroatoms in which the carbon atom constituting the ring represented by A¹ is substituted with the carbonyl carbon (>C═O) or the thiocarbonyl carbon (>C═S) to introduced into the ring (the carbonyl carbon (>C═O) described herein includes the carbonyl carbon specified in Formula (1-1)), and the number of heteroatoms of a substituent of A¹ are not included in the above-described number of heteroatoms.

A¹ may have a substituent, as the substituent, a halogen atom (preferably, a chlorine atom) an alkyl group (may be any of linear, branched, or cyclic; the number of carbon atoms is preferably 1 to 10 and more preferably 1 to 6), an aryl group (the number of carbon atoms is preferably 6 to 18 and more preferably 6 to 12), a heteroaryl group (the number of carbon atoms is preferably 5 to 18 and more preferably 5 or 6), or a silyl group (for example, an alkylsilyl group; an alkyl group in the alkylsilyl group may be any of linear, branched, or cyclic; the number of carbon atoms is preferably 1 to 4 and more preferably 1) is preferable.

A¹ may or may not exhibit aromaticity.

A¹ may have a monocyclic structure or a fused-ring structure, and is preferably a 5-membered ring, a 6-membered ring, or a fused ring containing at least any one of a 5-membered ring or a 6-membered ring. The number of rings forming the fused ring is preferably 1 to 4 and more preferably 1 to 3.

Usually, the ring represented by A¹ is preferably a ring used as an acidic nucleus (specifically, an acidic nucleus of a merocyanine coloring agent), and specific examples thereof include the following.
(a) 1,3-dicarbonyl nucleus: for example, 1,3-indandione nucleus, 1,3-cyclohexanedione, 5,5-dimethyl-1,3-cyclohexanedione, 1,3-dioxane-4,6-dione, and the like;
(b) pyrazolinone nucleus: for example, 1-phenyl-2-pyrazolin-5-one, 3-methyl-1-phenyl-2-pyrazolin-5-one, 1-(2-benzothiazolyl)-3-methyl-2-pyrazolin-5-one, 3-cyano-1-phenyl-2-pyrazolin-5-one, and the like;
(c) isoxazolinone nucleus: for example, 3-phenyl-2-isoxazolin-5-one, 3-methyl-2-isoxazolin-5-one, and the like;
   (d) oxindole nucleus: for example, 1-alkyl-2,3-dihydro-2-oxindole and the like;
(e) 2,4,6-trioxohexahydropyrimidine nucleus: for example, barbituric acid or 2-thibarbituric acid, derivatives thereof, and the like, examples of the derivatives include a 1-alkyl form such as 1-methyl and 1-ethyl, a 1,3-dialkyl form such as 1,3-dimethyl, 1,3-diethyl, and 1,3-dibutyl, a 1,3-diaryl form such as 1,3-diphenyl, 1,3-di(p-chlorophenyl), 1,3-di(p-ethoxycarbonylphenyl), a 1-alkyl-1-aryl form such as 1-ethyl-3-phenyl, and a 1,3-diheteroaryl form such as 1,3-di(2-pyridyl);
(f) 2-thio-2,4-thiazolidinedione nucleus: for example, rhodanine and derivatives thereof, and the like, examples of the derivatives include a 3-alkylrhodanine such as 3-methylrhodanine, 3-ethylrhodanine, and 3-allylrhodanine, a 3-arylrhodanine such as 3-phenylrhodanine, and a 3-heteroaryl rhodanine such as 3-(2-pyridyl)rhodanine;
(g) 2-thio-2,4-oxazolidinedione (2-thio-2,4-(3H,5H)-oxazoledione) nucleus: for example, 3-ethyl-2-thio-2,4-oxazolidinedione and the like;
(h) thianaphthenone nucleus: for example, 3(2H)-thianaphthenone-1,1-dioxide and the like;
(i) 2-thio-2,5-thiazolidinedione nucleus: for example, 3-ethyl-2-thio-2,5-thiazolidinedione and the like;
(j) 2,4-thiazolidinedione nucleus: for example, 2,4-thiazolidinedione, 3-ethyl-2,4-thiazolidinedione, 3-phenyl-2,4-thiazolidinedione, and the like;
(k) thiazolin-4-one nucleus: for example, 4-thiazolinone, 2-ethyl-4-thiazolinone, and the like;
(l) 2,4-imidazolidinedione (hydantoin) nucleus: for example, 2,4-imidazolidinedione, 3-ethyl-2,4-imidazolidinedione, and the like;
(m) 2-thio-2,4-imidazolidinedione (2-thiohydantoine) nucleus: for example, 2-thio-2,4-imidazolidinedione, 3-ethyl-2-thio-2,4-imidazolidinedione, and the like;
(n) imidazolin-5-one nucleus: for example, 2-propylmercapto-2-imidazolin-5-one and the like;
(o) 3,5-pyrazolidinedione nucleus: for example, 1,2-diphenyl-3,5-pyrazolidinedione, 1,2-dimethyl-3,5-pyrazolidinedione, and the like;
(p) benzothiophen-3(2H)-one nucleus: for example, benzothiophen-3(2H)-one, oxobenzothiophen-3(2H)-one, dioxobenzothiophen-3(2H)-one, and the like;
(q) indanone nucleus: for example, 1-indanone, 3-phenyl-1-indanone, 3-methyl-1-indanone, 3,3-diphenyl-1-indanone, 3-(dicyanomethylidene)indan-1-one, 3,3-dimethyl-1-indanone, and the like;
(r) benzofuran-3-(2H)-one nucleus: for example, benzofuran-3-(2H)-one and the like;
(s) 2,2-dihydrophenalene-1,3-dione nucleus and the like; and
(t) pyridone nucleus: for example, 3-cyano-1-ethyl-6-hydroxy-4-methyl-2-pyridone, 3-cyano-1-methyl-6-hydroxy-4-methyl-2-pyridone, 3-cyano-1-ethyl-6-hydroxy-4-trifluoromethyl-2-pyridone, 3-cyano-1-phenyl-6-hydroxy-4-trifluoromethyl-2-pyridone, and the like.

A¹ is preferably a ring having a group represented by Formula (AW), and more preferably a ring having a group represented by any one of Formulae (AW1) to (AW6) described later.

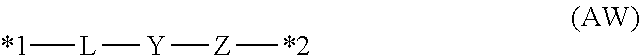

In Formula (AW), *1 represents a bonding position with the carbon atom in —C(═Z¹)-specified in Formula (1-1) (or Formula (1-1a)). *2 represents a bonding position with a carbon atom marked with * in Formula (1-1) (in other words, *2 represents a bonding position with a carbon atom bonded to the carbon atom to which R¹ in Formula (1) is directly bonded to form a double bond).

That is, in a case where A¹ is a ring having a group represented by Formula (AW), the compound represented by Formula (1), in which Y¹ is a group represented by Formula (1-1), (or the compound represented by Formula (1-1a) is a compound represented by Formula (1-1b).

The symbols used in Formula (1-1b) have the same meanings as the corresponding symbols used in Formula (1).

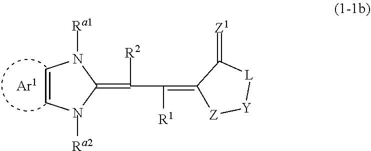

In Formula (AW), L represents a single bond or —NR^{L}—.

R^{L} represents a hydrogen atom or a substituent. Among these, R^{L} is preferably an alkyl group, an aryl group, or a heteroaryl group, and more preferably an alkyl group or an aryl group.

The above-described alkyl group and the above-described aryl group may have a substituent. As the substituent which may be included in the above-described aryl group, an alkyl group (for example, having 1 to 3 carbon atoms) is preferable.

Y represents —CR^{Y1}═CR^{Y2}—, —CS—NR^{Y3}—, —CO—, —CS—, —NR^{Y4}—, —N═CR^{Y5}—, —CO—NR^{Y6}—, or a 1,8-naphthalenedisyl group which may have a substituent; and among these, —CR^{Y1}═CR^{Y2}—, —CO—, or —N═CR^{Y5}— is preferable.

R^{Y1} to R^{Y6} each independently represent a hydrogen atom or a substituent. Among these, R^{Y1} to R^{Y6} are each independently preferably an alkyl group, a cyano group, an aryl group, or a heteroaryl group.

In addition, in a case where Y represents —CR^{Y1}═CR^{Y2}—, R^{Y1} and R^{Y2} may be bonded to each other to form a ring. Examples of the above-described ring include an aromatic ring; and specific examples thereof include a benzene ring, a naphthalene ring, an anthracene ring, and a pyridine ring. The above-described ring may further have a substituent, and such substituents may be bonded to each other to form a ring.

Z represents a single bond, —CO—, —S—, —SO₂—, —CR^{ZA}CR^{ZB}—, or —C(═CR^{Zc}R^{ZD})_; and among these, —CO—, —CR^{ZA}═CR^{ZB}—, or —C(═CR^{Zc}R^{ZD})_is preferable.

R^{ZA} to R^{ZD} each independently represent a hydrogen atom or a substituent.

R^{ZA} to R^{ZD} are each independently preferably an alkyl group, a cyano group, an aryl group, or a heteroaryl group. The above-described alkyl group may have a substituent, and for example, it is also preferable that the alkyl group is an alkyl group (for example, having 1 to 3 carbon atoms) having a halogen atom as a substituent, such as a trifluoromethyl group.

Examples of a more preferred form of the group represented by Formula (AW) include groups represented by any one of Formulae (AW1) to (AW6). Structures of the respective groups in Formulae (AW1) to (AW6) are as described above for the group represented by Formula (AW).

The group represented by any one of Formulae (AW1) to (AW6) is preferably a group represented by any one of Formulae (AW1) to (AW3).

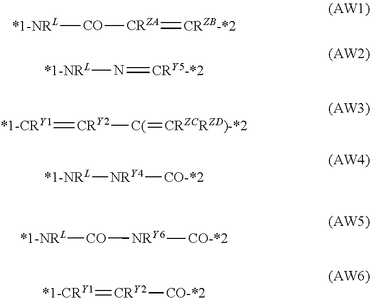

It is also preferable that A¹ has a group represented by Formula (AX). The group represented by Formula (AX) is more preferably a group represented by Formula (AY). In Formulae (AX) and (AY), *1 and *2 have the same meanings as *1 and *2 in Formula (AW).

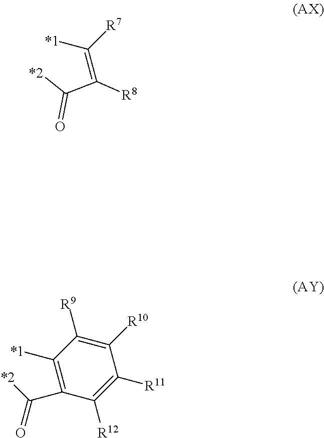

In Formula (AX), R⁷ and R⁸ each independently represent a hydrogen atom or a substituent.

It is preferable that R⁷ and R⁸ are bonded to each other to form a ring; and examples of the ring formed by bonding R⁷ and R⁸ to each other include an aromatic ring, and specific examples thereof include a benzene ring, a pyridazine ring, a pyrazine ring, and a pyridine ring.

It is also preferable that the ring formed by bonding R⁷ and R⁸ to each other further has a substituent. As the substituent, a halogen atom is preferable, and a chlorine atom is more preferable.

In addition, the substituents included in the ring formed by bonding R⁷ and R⁸ to each other may further be bonded to each other to form a ring (benzene ring or the like).

In Formula (AY), *1 and *2 have the same meanings as *1 and *2 in Formula (AX).

In Formula (AY), R⁹ to R¹² each independently represent a hydrogen atom or a substituent. Among these, R⁹ to R¹² are each independently preferably a hydrogen atom or a halogen atom, and more preferably a hydrogen atom or a chlorine atom.

R⁹ and R¹⁰, R¹⁰ and R¹¹, or R¹¹ and R¹² may be bonded to each other to form a ring.

Examples of the above-described ring include an aromatic ring, and specifically, a benzene ring is preferable.

Among these, it is preferable that R¹⁰ and R¹¹ are bonded to each other to form a ring.

The above-described ring may be further substituted with a substituent. Such substituents included in the ring may be bonded to each other to form a ring. In addition, if possible, the substituent included in the ring and one or more of R⁹ to R¹² may be bonded to each other to form one or more rings.

A group formed by bonding the substituents included in the ring to each other may be a single bond.

In Formula (1-2), R^{b1} and R^{b2} each independently represent a cyano group or —COOR^{b3}.

R^{b3} represents an alkyl group which may have a substituent, an aryl group (phenyl group or the like) which may have a substituent, or a heteroaryl group which may have a substituent.

In Formula (1), R¹ and R² each independently represent a hydrogen atom or a substituent. R¹ and R² are preferably a hydrogen atom.

R^{a1} and R^{a2} each independently represent an aryl group which may have a substituent, —C(R^{L1})(R^{L2})(R^{L3}), or a heteroaryl group which may have a substituent. Here, R^{a1} and R² represent groups different from each other.

The above-described aryl group is preferably a phenyl group, a naphthyl group, or a fluorenyl group, and more preferably a phenyl group or a naphthyl group.

In a case where the above-described aryl group is a phenyl group, the phenyl group preferably has a substituent, and the substituent is preferably an alkyl group (preferably having 1 to 3 carbon atoms).

In a case where the above-described aryl group is a phenyl group, the number of substituents included in the phenyl group is preferably 1 to 5 and more preferably 2 or 3.

R^{L1} to R^{L3} in —C(R^{L1})(R^{L2})(R^{L3}) each independently represent an alkyl group which may have a substituent, an aryl group which may have a substituent, a heteroaryl group which may have a substituent, or a hydrogen atom, and two or more of R^{L1} to R^{L3} each independently represent an alkyl group which may have a substituent, an aryl group which may have a substituent, or a heteroaryl group which may have a substituent.

The alkyl group which may have a substituent, the aryl group which may have a substituent, and the heteroaryl group which may have a substituent, which are represented by R^{L1} to R^{L3}, may be bonded to each other to form a ring.

Examples of the above-described ring include a ring formed by bonding alkyl groups which may have a substituent to each other. A substituent in the aryl group which may have a substituent (or the heteroaryl group which may have a substituent) and a substituent in the alkyl group which may have a substituent may be bonded to each other to form a ring.

A substituent in the aryl group which may have a substituent (or the heteroaryl group which may have a substituent) and a substituent in another aryl group which may have a substituent (or another heteroaryl group which may have a substituent) may be bonded to each other to form a ring.

A substituent in the ring formed as described above, and another alkyl group which may have a substituent, a substituent in another aryl group which may have a substituent, or a substituent in another heteroaryl group which may have a substituent may be bonded to form a ring.

As described above, a group formed by bonding the above-described substituent and substituent (for example, the substituent in the aryl group which may have a substituent and the substituent in the heteroaryl group which may have a substituent) to each other may be a single bond.

In a case where the alkyl group which may have a substituent, the aryl group which may have a substituent, and the heteroaryl group which may have a substituent, which are represented by R^{L1} to R^{L3}, may be bonded to each other to form a ring, —C(R^{L1})(R^{L2})(R^{L3}) is preferably a group other than the aryl group and the heteroaryl group.

The alkyl group represented by R^{L1} to R^{L3} may be linear, branched, or cyclic. In the alkyl group represented by R^{L1} to R^{L3}, two alkyl groups may be bonded to each other to form a ring.

For example, the alkyl group represented by R^{L1} and the alkyl group represented by R^{L2} may be bonded to each other to form a ring. Furthermore, a substituent included in the ring (a monocyclic cycloalkane ring or the like), which is formed by bonding the alkyl group represented by R^{L1} and the alkyl group represented by R^{L2} to each other, and the alkyl group represented by R^{L3} may be bonded to each other to form a polycyclic ring (a polycyclic cycloalkane ring or the like).

That is, —C(R^{L1})(R^{L2})(R^{L3}) may be a cycloalkyl group (preferably, a cyclohexyl group) which may have a substituent. The number of ring members in the above-described cycloalkyl group is preferably 3 to 12, more preferably 5 to 8, and still more preferably 6.

The above-described cycloalkyl group may be a monocyclic ring (a cyclohexyl group or the like) or a polycyclic ring (1-adamantyl group or the like).

The above-described cycloalkyl group preferably has a substituent. In a case where the above-described cycloalkyl group has a substituent, a carbon atom adjacent to a carbon atom directly bonded to the nitrogen atom specified in General Formula (1) (that is, the “C” atom specified in “—C(R^{L1})(R^{L2})(R^{L3})”) preferably has the substituent.

Examples of the substituent which may be included in the above-described cycloalkyl group includes an alkyl group (preferably having 1 to 3 carbon atoms).

Substituents included in the above-described cycloalkyl group may be bonded to each other to form a ring, and the ring formed by bonding the substituents to each other may be a ring other than a cycloalkane ring.

R^{a1} and R^{a2} are each independently preferably a group represented by Formula (X), —C(R^{L1})(R^{L2})(R^{L3}), a polycyclic aryl group which may have a substituent, or a polycyclic heteroaryl group which may have a substituent; and more preferably a group represented by Formula (X), —C(R^{L1})(R^{L2})(R^{L3}), or a polycyclic aryl group which may have a substituent.

The group represented by Formula (X) is preferably a group represented by Formula (Z) described later, and more preferably a group represented by Formula (ZB) described later.

The group represented by Formula (X) is shown below. * represents a bonding position. The aromatic ring of B¹ is directly bonded to the nitrogen atom specified in Formula (1).

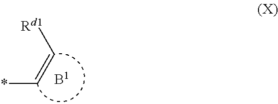

In Formula (X), B¹ represents a monocyclic aromatic ring which may have a substituent other than R^{d1}.

R^{d1} represents an alkyl group, a silyl group, an alkoxy group, an alkylthio group, a cyano group, a halogen atom, an aryl group, a heteroaryl group, an alkenyl group, or an alkynyl group.

Each group represented by R^{d1} may further have a substituent, if possible. The definition of the substituent is the same as that of the above-described substituent W.

Examples of the substituent include an alkyl group, an aryl group, a heteroaryl group, a silyl group, a halogen atom, and a cyano group. The substituent included in R^{d1} and the substituent included in B¹ may be bonded to each other to form a non-aromatic ring.

Examples of the above-described monocyclic aromatic ring include a monocyclic aromatic hydrocarbon ring and a monocyclic aromatic heterocyclic ring. Examples of the aromatic hydrocarbon ring include a benzene ring. Examples of the aromatic heterocyclic ring include a pyrrole ring, a furan ring, a thiophene ring, an imidazole ring, and an oxazole ring.

Among these, from the viewpoint that heat resistance of the photoelectric conversion element is more excellent, an aromatic hydrocarbon ring is preferable, and a benzene ring is more preferable.

The number of carbon atoms in the alkyl group represented by R^{d1} is preferably 1 to 12, more preferably 1 to 6, and still more preferably 1 to 3.

In addition, the above-described alkyl group may be —CH(R^{d3})(R^{d4}) or —C(R^{d3})(R^{d4})(R^{d5}). R^{d3} to R^{d5} each independently represent an aryl group, an alkyl group (for example, having 1 to 3 carbon atoms), or a heteroaryl group.

Examples of the silyl group represented by R^{d1} include a group represented by —Si(R^{p})(R^{q})(R^{r}). R^{p} to R^{r} each independently represent a hydrogen atom or a substituent.

Examples of the substituent represented by R^{p} to R^{r} include an alkyl group (may be any of linear, branched, or cyclic; the number of carbon atoms is preferably 1 to 4), an aryl group, and a heteroaryl group. These groups may further have a substituent.

The number of carbon atoms in the silyl group represented by R^{d1} is preferably 1 to 12, more preferably 1 to 6, and still more preferably 3.

The number of carbon atoms in the alkoxy group represented by R^{d1} is preferably 1 to 12, more preferably 1 to 6, and still more preferably 1 to 3.

The number of carbon atoms in the alkylthio group represented by R^{d1} is preferably 1 to 12, more preferably 1 to 6, and still more preferably 1 to 3.

Examples of the halogen atom represented by R^{d1} include a fluorine atom, an iodine atom, a bromine atom, and a chlorine atom.

The number of carbon atoms in the alkenyl group represented by R^{d1} is preferably 2 to 12, more preferably 2 to 6, and still more preferably 2 or 3.

The number of carbon atoms in the alkynyl group represented by R^{d1} is preferably 2 to 12, more preferably 2 to 6, and still more preferably 2 or 3.

The above-described group represented by Formula (X) is preferably a group represented by Formula (Z).

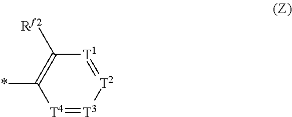

In Formula (Z), R¹ represents an alkyl group, a silyl group, an alkoxy group, an alkylthio group, a cyano group, a halogen atom, an aryl group, a heteroaryl group, an alkenyl group, or an alkynyl group. R^{f2} has the same meaning as R^{d1} in Formula (X), and preferred aspects thereof are also the same.

In Formula (Z), T¹ to T⁴ each independently represent —CR^{e12}═ or a nitrogen atom (═N—). R^{e12} represents a hydrogen atom or a substituent. In a case where a plurality of R^{e12}'s in Formula (Z), R^{e12}'s may be the same or different from each other. In addition, R^{e12} and R^{f2} may be bonded to each other to form a non-aromatic ring.

It is preferable that “at least one of T¹ to T⁴ represents —CR^{e12}═ and at least one of R^{e12}'s represents a substituent”, and it is more preferable that “at least T⁴ represents —CR^{e12}═ and R^{e12} represents an alkyl group, an aryl group, or a heteroaryl group”. In addition, an aspect in which “at least T⁴ represents —CR^{e12}═ and R^{e12} is —CH(R^{d3})(R^{d4}) or —C(R^{d3})(R^{d4})(R^{d5})” may be used.

The —CH(R^{d3})(R^{d4}) and the —C(R^{d3})(R^{d4})(R^{d5}) will be described below.

The definition of the substituent is the same as that of the above-described substituent W. Examples of the substituent include an alkyl group, an aryl group, a heteroaryl group, a silyl group, a halogen atom, and a cyano group. In addition, these groups may further have a substituent (for example, a halogen atom such as a fluorine atom).

The number of carbon atoms in the alkyl group represented by R^{e12} is preferably 1 to 12, more preferably 1 to 6, and still more preferably 1 to 3. In addition, the above-described alkyl group may be —CH(R^{d3})(R^{d4}) or —C(R^{d3})(R^{d4})(R^{d5}). R^{d3} to R^{d5} each independently represent an aryl group, an alkyl group (for example, having 1 to 3 carbon atoms), or a heteroaryl group.

Examples of the silyl group represented by R^{e12} include the silyl group described as the silyl group represented by R^{d1}.

Examples of the halogen atom represented by R^{e12} include a fluorine atom, an iodine atom, a bromine atom, and a chlorine atom.

As the group represented by Formula (X), a group represented by Formula (ZB) is more preferable.

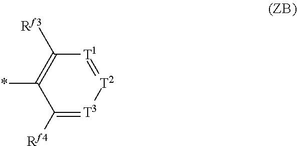

In Formula (ZB), T¹ to T³ each independently represent —CR^{e12}═ or a nitrogen atom. R^{e12} represents a hydrogen atom or a substituent.

R^{e12} in Formula (ZB) is the same as R^{e12} in Formula (Z).

R^{f3} and R^{f4} each independently represent an alkyl group, an aryl group, or a heteroaryl group. The alkyl group is preferably a group represented by —CH(R^{d3})(R^{d4}) or a group represented by —C(R^{d3})(R^{d4})(R^{d5}). R^{d3} to R^{d5} each independently represent an alkyl group (for example, having 1 to 3 carbon atoms), an aryl group, or a heteroaryl group.

These groups may further have a substituent, if possible. The definition of the substituent is the same as that of the above-described substituent W. Examples of the substituent include an alkyl group, an aryl group, a heteroaryl group, a silyl group, a halogen atom, and a cyano group.
* represents a bonding position.

The number of rings constituting the polycyclic aryl group which may have a substituent and the polycyclic heteroaryl group which may have a substituent is 2 or more, preferably 2 to 4, more preferably 2 to 3, and still more preferably 2.

The substituent which may be included in the polycyclic aryl group which may have a substituent or in the polycyclic heteroaryl group which may have a substituent may contain a non-aromatic ring.

As the polycyclic aryl group which may have a substituent, for example, a naphthyl group which may have a substituent is preferable.

The combination of R^{a1} and R^{a2} is not particularly limited as long as they represent groups different from each other, but it is preferable that R^{a1} and R^{a2} represent aryl groups different from each other. The “groups from each other” means that the structures of the groups are different from each other.

Among these, R^{a1} and R^{a2} are preferably the group represented by Formula (X), more preferably the group represented by Formula (Z), and still more preferably the group represented by Formula (ZB).

In Formula (1), Ar¹ represents an aromatic ring which may have a substituent.

The aromatic ring may be a monocyclic ring or a polycyclic ring.

Examples of the aromatic ring include an aromatic hydrocarbon ring and an aromatic heterocyclic ring. Examples of the aromatic hydrocarbon ring include a benzene ring, a naphthalene ring, an anthracene ring, and a phenanthrene ring. Examples of the aromatic heterocyclic ring include a quinoxaline ring, a pyrazine ring, a pyrrole ring, a furan ring, a thiophene ring, an imidazole ring, and an oxazole ring. These rings may be further fused with another ring (which may be a non-aromatic ring).

Among these, Ar is preferably an aromatic heterocyclic ring, and more preferably a quinoxaline ring or a pyrazine ring.

As the substituent included in the aromatic ring represented by Ar¹, an alkyl group or an alkoxy group is preferable.

### Compound Represented by Formula (2)

The compound represented by Formula (1) is preferably a compound represented by Formula (2), and more preferably a compound represented by Formula (3).

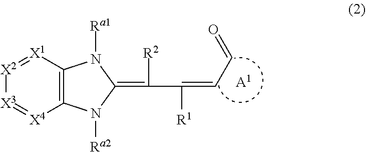

A¹ in Formula (2) has the same meaning as A¹ in Formula (1-1) (or Formula (1-1a)), and preferred aspects thereof are also the same.

R¹ and R² in Formula (2) have the same meanings as R¹ and R² in Formula (1), and preferred aspects thereof are also the same.

R^{a1} and R^{a2} in Formula (2) have the same meanings as R^{a1} and R^{a2} in Formula (1), and preferred aspects thereof are also the same.

In Formula (2), X¹ to X⁴ each independently represent a nitrogen atom (—N═) or —CR^{c1}—.

R^{c1} represents a hydrogen atom or a substituent.

It is preferable that at least two of X¹ to X⁴ are nitrogen atoms, it is more preferable that at least X¹ and X⁴ are nitrogen atoms, and it is still more preferable that only X¹ and X⁴ are nitrogen atoms.

In a case of a plurality of R^{c1}'s, the plurality of R^{c1}'s may be bonded to each other to form a ring. The ring formed by bonding the plurality of R^{c1}'s to each other is preferably an aromatic ring, and more preferably a benzene ring or a pyridine ring. The ring formed by bonding the plurality of R^{c1}'s to each other may further have a substituent.

Compound represented by Formula (3)

The compound represented by Formula (1) is more preferably a compound represented by Formula (3).

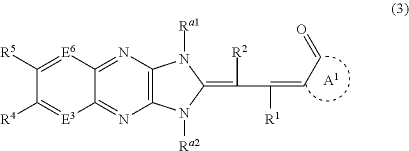

A¹ in Formula (3) has the same meaning as A¹ in Formula (1-1) (or Formula (1-1a)), and preferred aspects thereof are also the same.

R¹ and R² in Formula (3) have the same meanings as R¹ and R² in Formula (1), and preferred aspects thereof are also the same.

E³ represents a nitrogen atom (—N═) or —CR³═.

E⁶ represents a nitrogen atom (—N═) or —CR⁶═.

E³ and E⁶ preferably have “an aspect in which E³ is —CR³=E⁶ is —CR⁶═”, “an aspect in which E³ is —N═ and E⁶ is —CR⁶═”, or “an aspect in which E³ is —CR³═ and E⁶ is —N═”, and more preferably have “an aspect in which E³ is —CR³═ and E⁶ is —CR⁶═”.

R³ to R⁶ each independently represent a hydrogen atom or a substituent.

R³ to R⁶ are each independently preferably a hydrogen atom, an alkoxy group, a silyl group, a chlorine atom, a fluorine atom, a cyano group, or an alkyl group; and more preferably a hydrogen atom, an alkoxy group having 1 to 3 carbon atoms in an alkyl group moiety, a chlorine atom, a fluorine atom, a cyano group, or an alkyl group having 1 to 4 carbon atoms.

In R³ to R⁶, the number of R³ to R⁶ representing a substituent is preferably 0 to 2. In a case where one or more of R³ to R⁶ represent a substituent, it is preferable that R⁴ and/or R⁵ represent a substituent.

R³ and R⁴ in a case where E³ is —CR³═, R⁴ and R⁵, or R⁵ and R⁶ in a case where E⁶ is —CR⁶═ may be each independently bonded to each other to form a ring. The ring formed by bonding R³ and R⁴, R⁴ and R⁵, or R⁵ and R⁶ to each other may be a monocyclic ring or a polycyclic ring, may be aromatic or non-aromatic, and may have a substituent.

The number of ring member atoms in the above-described ring is preferably 5 to 12 and more preferably 5 to 7.

For example, it is also preferable that R³ and R⁴, R⁴ and R⁵, or R⁵ and R⁶ (preferably R⁴ and R⁵) are bonded to each other to form a benzene ring which may further have a substituent. In this case, the benzene ring (the benzene ring which may further have a substituent) is fused to the ring including E³ and E⁶.

R^{a1} and R^{a2} in Formula (3) have the same meanings as R^{a1} and R^{a2} in Formula (1), and preferred aspects thereof are also the same.

### Compound Represented by Formula (4)

The compound represented by Formula (1) may be a compound represented by Formula (4).

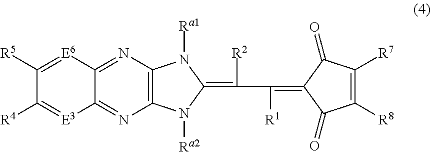

R¹ and R² in Formula (4) have the same meanings as R¹ and R² in Formula (1), and preferred aspects thereof are also the same.

E³ and E⁶ in Formula (4) have the same meanings as E³ and E⁶ in Formula (3), and preferred aspects thereof are also the same.

R³ to R⁶ in Formula (4) have the same meanings as R³ to R⁶ in Formula (3), and preferred aspects thereof are also the same.

R⁷ and R⁸ in Formula (4) have the same meanings as R⁷ and R⁸ in Formula (AX), and preferred aspects thereof are also the same.

R^{a1} and R^{a2} in Formula (4) have the same meanings as R^{a1} and R^{a2} in Formula (1), and preferred aspects thereof are also the same.

### Compound Represented by Formula (4-2)

Examples of a suitable aspect of the compound represented by Formula (4) include a compound represented by Formula (4-2).

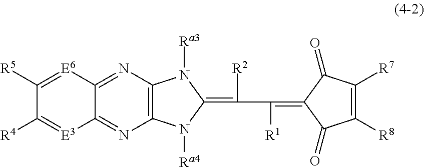

R¹ and R² in Formula (4-2) have the same meanings as R¹ and R² in Formula (1), and preferred aspects thereof are also the same.

E³ and E⁶ in Formula (4-2) have the same meanings as E³ and E⁶ in Formula (3), and preferred aspects thereof are also the same.

R³ to R⁶ in Formula (4-2) have the same meanings as R³ to R⁶ in Formula (3), and preferred aspects thereof are also the same.

R⁷ and R⁸ in Formula (4-2) have the same meanings as R⁷ and R⁸ in Formula (AX), and preferred aspects thereof are also the same.

R^{a3} and R^{a4} in Formula (4-2) each independently represent a group represented by Formula (X), —C(R^{L1})(R^{L2})(R^{L3}), a polycyclic aryl group which may have a substituent, or a polycyclic heteroaryl group which may have a substituent. Here, R^{a3} and R^{a4} represent groups different from each other.

The group represented by Formula (X), —C(R^{L1})(R^{L2})(R^{L3}), the polycyclic aryl group which may have a substituent, and the polycyclic heteroaryl group which may have a substituent, in Ras and R^{a4} of Formula (4-2), have the same meanings as the group represented by Formula (X), —C(R^{L1})(R^{L2})(R^{L3}), the polycyclic aryl group which may have a substituent, and the polycyclic heteroaryl group which may have a substituent, which are respectively described for R^{a1} and R^{a2} in Formula (1); and preferred aspects thereof are also the same.

### Compound Represented by Formula (5)

The compound represented by Formula (1) may be a compound represented by Formula (5).

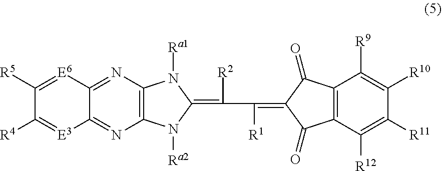

R¹ and R² in Formula (5) have the same meanings as R¹ and R² in Formula (1), and preferred aspects thereof are also the same.

E³ and E⁶ in Formula (5) have the same meanings as E³ and E⁶ in Formula (3), and preferred aspects thereof are also the same.

R³ to R⁶ in Formula (5) have the same meanings as R³ to R⁶ in Formula (3), and preferred aspects thereof are also the same.

R⁹ to R¹² in Formula (5) have the same meanings as R⁹ to R¹² in Formula (AY), and preferred aspects thereof are also the same.

R^{a1} and R^{a2} in Formula (5) have the same meanings as Rai and R^{a2} in Formula (1), and preferred aspects thereof are also the same.

Hereinafter, the compound represented by Formula (1) will be exemplified.

In a case where the compounds exemplified below are applied to Formula (1), the compounds represented by Formula (1) exemplified below include all geometric isomers which can be distinguished based on a C═C double bond constituted by a carbon atom to which R¹ bonds and a carbon atom adjacent thereto. That is, both the cis isomer and the trans isomer, which are distinguished based on the above-described C═C double bond, are included in the compounds represented by Formula (1) exemplified below.

In the following examples, Me represents a methyl group and Ph represents a phenyl group.

In addition, in addition to the exemplary compounds, examples of the compound represented by Formula (1) include compounds described in paragraphs [0091] to [0095] of WO2020/013246A, the contents of which are incorporated herein by reference.

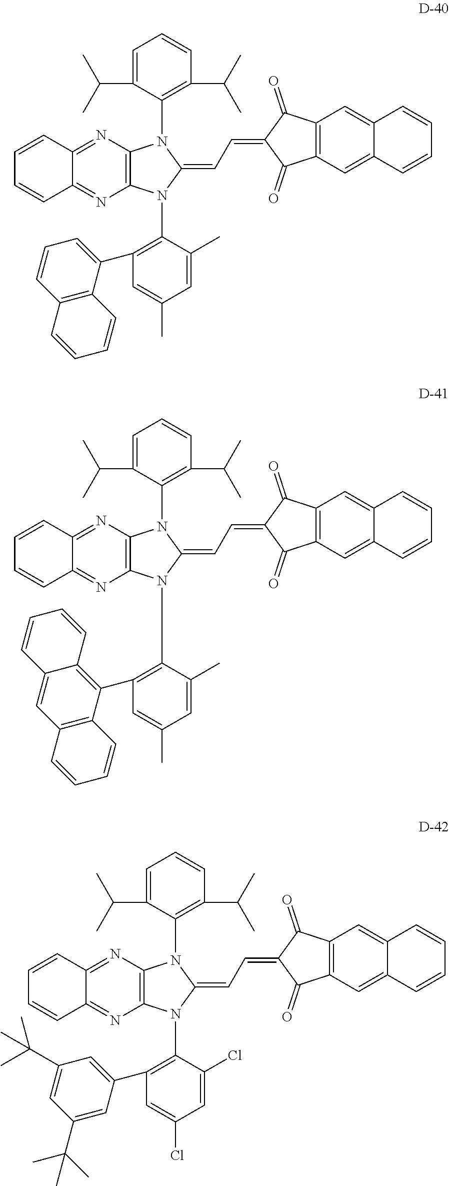 [SMILES: Cc1cc(C)c(N2/C(=C/C=C3C(=O)c4cc5ccccc5cc4C3=O)N(c3c(C(C)C)cccc3C(C)C)c3nc4ccccc4nc32)c(-c2cccc3ccccc23)c1 ; Cc1cc(C)c(N2/C(=C/C=C3C(=O)c4cc5ccccc5cc4C3=O)N(c3c(C(C)C)cccc3C(C)C)c3nc4ccccc4nc32)c(-c2c3ccccc3cc3ccccc23)c1 ; CC(C)c1cccc(C(C)C)c1N1/C(=C\C=C2C(=O)c3cc4ccccc4cc3C2=O)N(c2c(Cl)cc(Cl)cc2-c2cc(C(C)(C)C)cc(C(C)(C)C)c2)c2nc3ccccc3nc21]

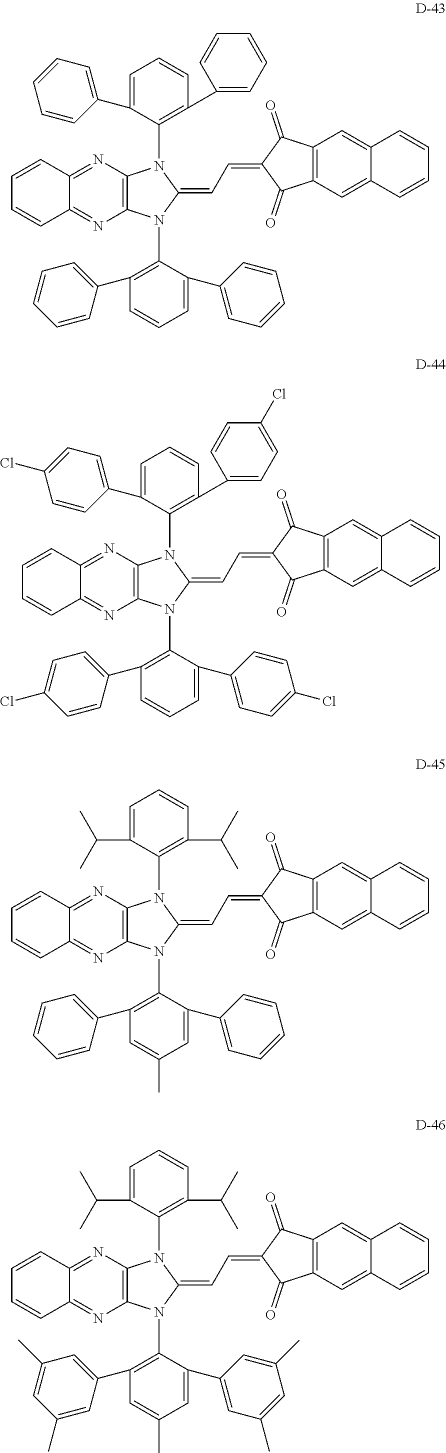 [SMILES: O=C1C(=CC=C2N(c3c(-c4ccccc4)cccc3-c3ccccc3)c3nc4ccccc4nc3N2c2c(-c3ccccc3)cccc2-c2ccccc2)C(=O)c2cc3ccccc3cc21 ; O=C1C(=CC=C2N(c3c(-c4ccc(Cl)cc4)cccc3-c3ccc(Cl)cc3)c3nc4ccccc4nc3N2c2c(-c3ccc(Cl)cc3)cccc2-c2ccc(Cl)cc2)C(=O)c2cc3ccccc3cc21 ; Cc1cc(-c2ccccc2)c(N2/C(=C/C=C3C(=O)c4cc5ccccc5cc4C3=O)N(c3c(C(C)C)cccc3C(C)C)c3nc4ccccc4nc32)c(-c2ccccc2)c1 ; Cc1cc(C)cc(-c2cc(C)cc(-c3cc(C)cc(C)c3)c2N2/C(=C/C=C3C(=O)c4cc5ccccc5cc4C3=O)N(c3c(C(C)C)cccc3C(C)C)c3nc4ccccc4nc32)c1]

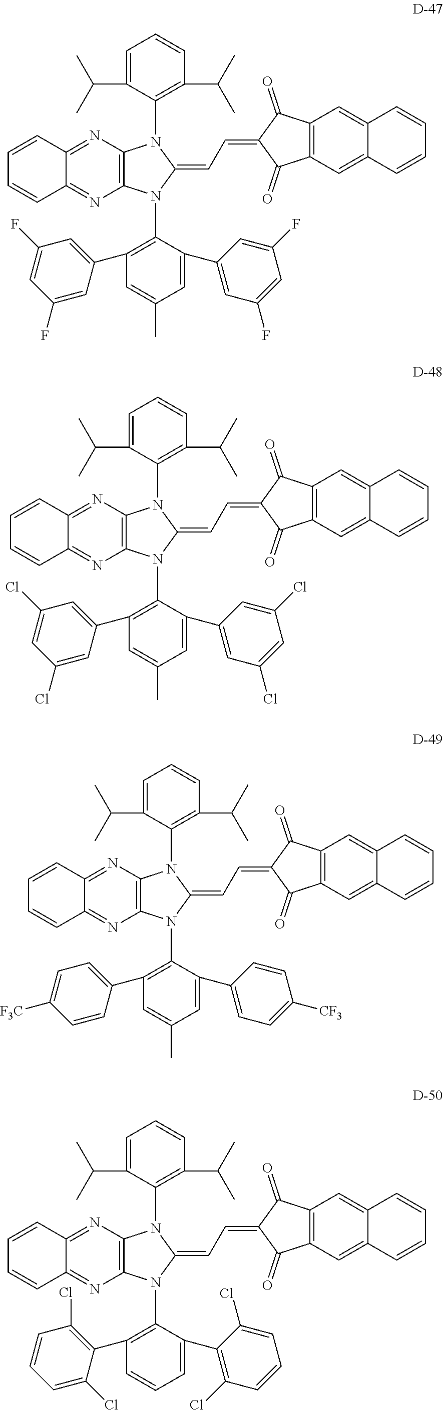 [SMILES: Cc1cc(-c2cc(F)cc(F)c2)c(N2/C(=C/C=C3C(=O)c4cc5ccccc5cc4C3=O)N(c3c(C(C)C)cccc3C(C)C)c3nc4ccccc4nc32)c(-c2cc(F)cc(F)c2)c1 ; Cc1cc(-c2cc(Cl)cc(Cl)c2)c(N2/C(=C/C=C3C(=O)c4cc5ccccc5cc4C3=O)N(c3c(C(C)C)cccc3C(C)C)c3nc4ccccc4nc32)c(-c2cc(Cl)cc(Cl)c2)c1 ; Cc1cc(-c2ccc(C(F)(F)F)cc2)c(N2/C(=C/C=C3C(=O)c4cc5ccccc5cc4C3=O)N(c3c(C(C)C)cccc3C(C)C)c3nc4ccccc4nc32)c(-c2ccc(C(F)(F)F)cc2)c1 ; CC(C)c1cccc(C(C)C)c1N1/C(=C\C=C2C(=O)c3cc4ccccc4cc3C2=O)N(c2c(-c3c(Cl)cccc3Cl)cccc2-c2c(Cl)cccc2Cl)c2nc3ccccc3nc21]

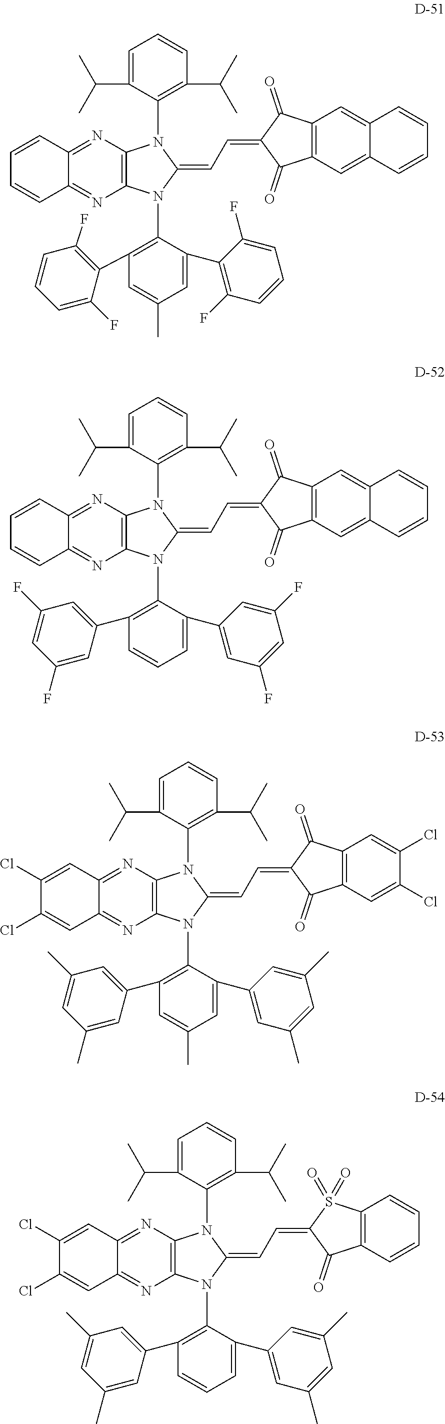 [SMILES: Cc1cc(-c2c(F)cccc2F)c(N2/C(=C/C=C3C(=O)c4cc5ccccc5cc4C3=O)N(c3c(C(C)C)cccc3C(C)C)c3nc4ccccc4nc32)c(-c2c(F)cccc2F)c1 ; CC(C)c1cccc(C(C)C)c1N1/C(=C\C=C2C(=O)c3cc4ccccc4cc3C2=O)N(c2c(-c3cc(F)cc(F)c3)cccc2-c2cc(F)cc(F)c2)c2nc3ccccc3nc21 ; Cc1cc(C)cc(-c2cc(C)cc(-c3cc(C)cc(C)c3)c2N2/C(=C/C=C3C(=O)c4cc(Cl)c(Cl)cc4C3=O)N(c3c(C(C)C)cccc3C(C)C)c3nc4cc(Cl)c(Cl)cc4nc32)c1 ; Cc1cc(C)cc(-c2cccc(-c3cc(C)cc(C)c3)c2N2/C(=C/C=C3\C(=O)c4ccccc4S3(=O)=O)N(c3c(C(C)C)cccc3C(C)C)c3nc4cc(Cl)c(Cl)cc4nc32)c1]

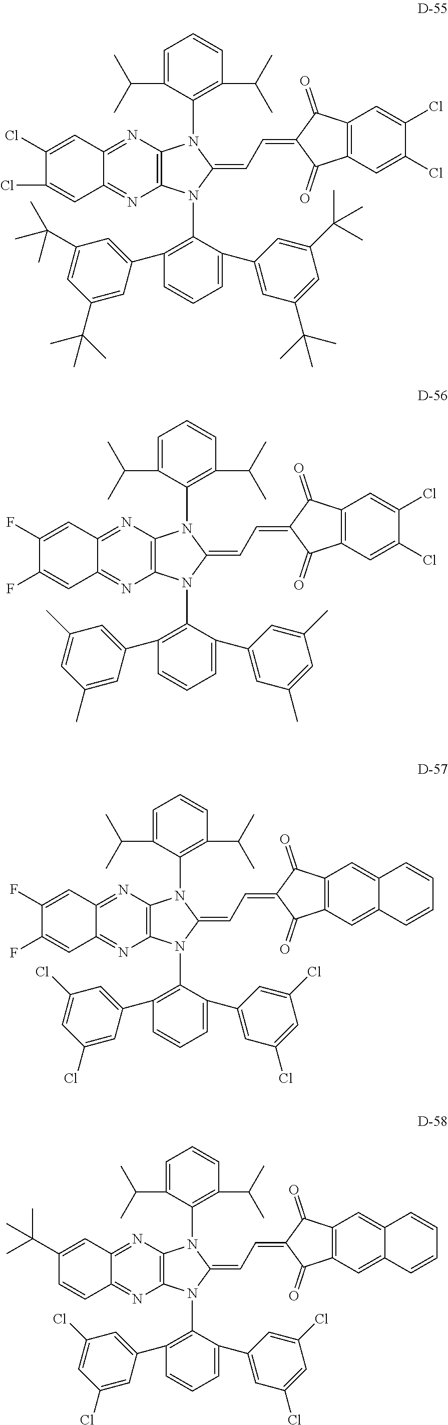

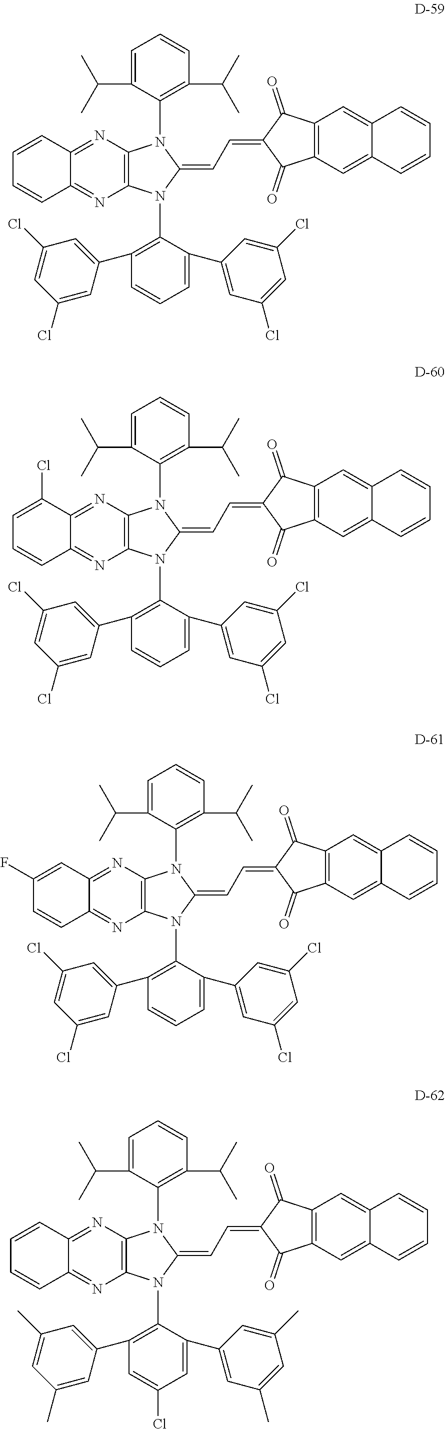 [SMILES: CC(C)c1cccc(C(C)C)c1N1/C(=C\C=C2C(=O)c3cc4ccccc4cc3C2=O)N(c2c(-c3cc(Cl)cc(Cl)c3)cccc2-c2cc(Cl)cc(Cl)c2)c2nc3ccccc3nc21 ; CC(C)c1cccc(C(C)C)c1N1/C(=C\C=C2C(=O)c3cc4ccccc4cc3C2=O)N(c2c(-c3cc(Cl)cc(Cl)c3)cccc2-c2cc(Cl)cc(Cl)c2)c2nc3cccc(Cl)c3nc21 ; CC(C)c1cccc(C(C)C)c1N1/C(=C\C=C2C(=O)c3cc4ccccc4cc3C2=O)N(c2c(-c3cc(Cl)cc(Cl)c3)cccc2-c2cc(Cl)cc(Cl)c2)c2nc3ccc(F)cc3nc21 ; Cc1cc(C)cc(-c2cc(Cl)cc(-c3cc(C)cc(C)c3)c2N2/C(=C/C=C3C(=O)c4cc5ccccc5cc4C3=O)N(c3c(C(C)C)cccc3C(C)C)c3nc4ccccc4nc32)c1]

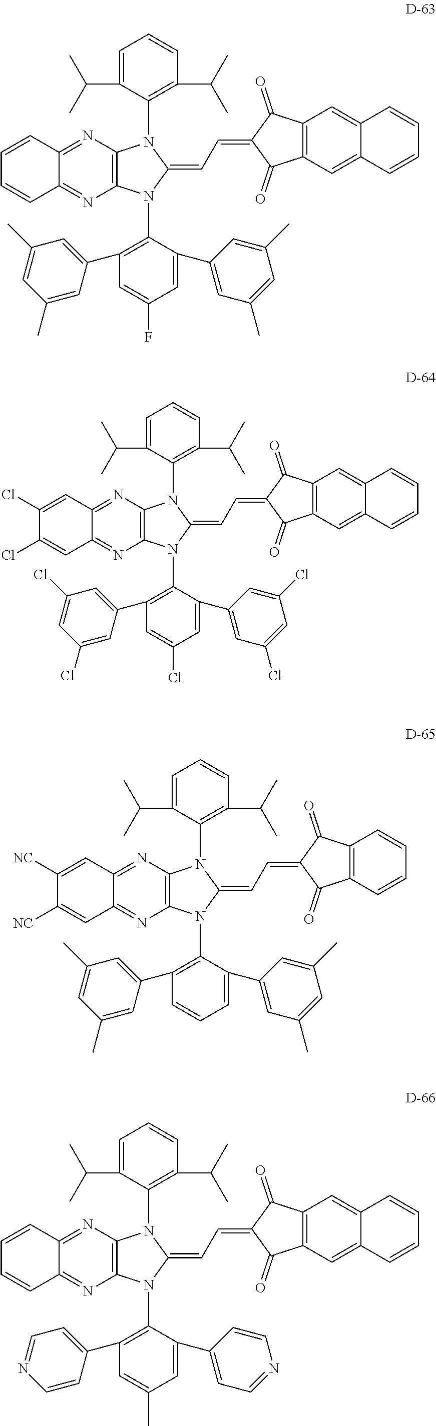 [SMILES: Cc1cc(C)cc(-c2cc(F)cc(-c3cc(C)cc(C)c3)c2N2/C(=C/C=C3C(=O)c4cc5ccccc5cc4C3=O)N(c3c(C(C)C)cccc3C(C)C)c3nc4ccccc4nc32)c1 ; CC(C)c1cccc(C(C)C)c1N1/C(=C\C=C2C(=O)c3cc4ccccc4cc3C2=O)N(c2c(-c3cc(Cl)cc(Cl)c3)cc(Cl)cc2-c2cc(Cl)cc(Cl)c2)c2nc3cc(Cl)c(Cl)cc3nc21 ; Cc1cc(C)cc(-c2cccc(-c3cc(C)cc(C)c3)c2N2/C(=C/C=C3C(=O)c4ccccc4C3=O)N(c3c(C(C)C)cccc3C(C)C)c3nc4cc(C#N)c(C#N)cc4nc32)c1 ; Cc1cc(-c2ccncc2)c(N2/C(=C/C=C3C(=O)c4cc5ccccc5cc4C3=O)N(c3c(C(C)C)cccc3C(C)C)c3nc4ccccc4nc32)c(-c2ccncc2)c1]

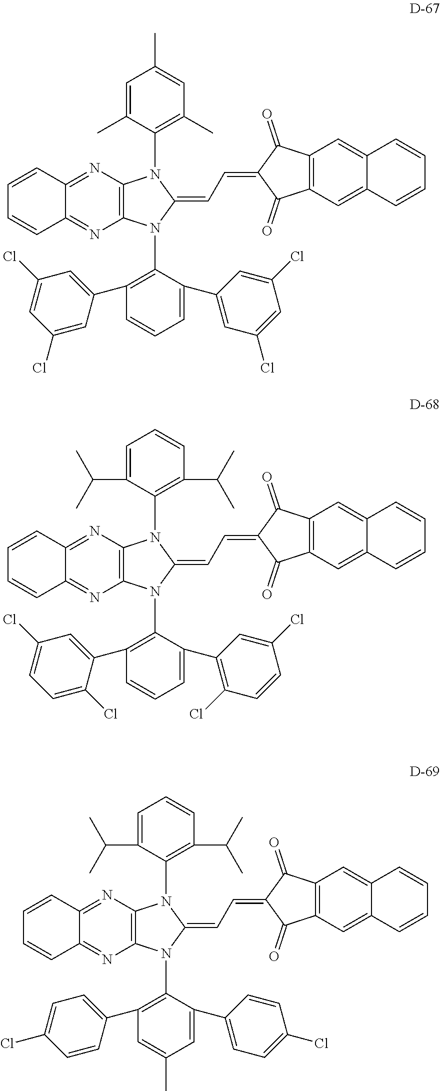 [SMILES: Cc1cc(C)c(N2/C(=C\C=C3C(=O)c4cc5ccccc5cc4C3=O)N(c3c(-c4cc(Cl)cc(Cl)c4)cccc3-c3cc(Cl)cc(Cl)c3)c3nc4ccccc4nc32)c(C)c1 ; CC(C)c1cccc(C(C)C)c1N1/C(=C\C=C2C(=O)c3cc4ccccc4cc3C2=O)N(c2c(-c3cc(Cl)ccc3Cl)cccc2-c2cc(Cl)ccc2Cl)c2nc3ccccc3nc21 ; Cc1cc(-c2ccc(Cl)cc2)c(N2/C(=C/C=C3C(=O)c4cc5ccccc5cc4C3=O)N(c3c(C(C)C)cccc3C(C)C)c3nc4ccccc4nc32)c(-c2ccc(Cl)cc2)c1]

A molecular weight of the first compound is not particularly limited, but is preferably 300 to 1,200. In a case where the molecular weight is 1,200 or less, the vapor deposition temperature does is not high, and the decomposition of the compound hardly occurs. In a case where the molecular weight is 300 or more, the glass transition point of a deposited film is not low, and heat resistance of the photoelectric conversion element is improved.

A maximal absorption wavelength of the first compound is preferably 490 to 600 nm, more preferably 510 to 590 nm, and still more preferably 530 to 590 nm.

From the viewpoint of matching energy levels with the n-type semiconductor material described later, the first compound is preferably a compound having an ionization potential of 5.0 to 6.2 eV in a single film, more preferably a compound having an ionization potential of 5.2 to 6.1 eV, and still more preferably a compound having an ionization potential of 5.4 to 6.0 eV.

In the present specification, the ionization potential is a value measured by AC-2 of a photoelectron spectrometer manufactured by RIKEN KEIKI CO., LTD. for the single film of the compound.

A content of the first compound with respect to the entire photoelectric conversion film (=(Film thickness of first compound in terms of single layer/Film thickness of entire photoelectric conversion film)×100) is preferably 5% to 70% by volume, more preferably 10% to 50% by volume, and still more preferably 15% to 40% by volume.

### (Second Compound)

Next, the second compound will be described.

The second compound is a compound different from the first compound, and is a compound having a maximal absorption wavelength which satisfies a relationship of the expression (X), which represents a relationship between the maximal absorption wavelength λ1 of the first compound and the maximal absorption wavelength λ2 of the second compound.

$$- 20 nm \leq λ 1 - λ 2 \leq 20 nm \qquad Expression ( X )$$

Among these, it is preferable that the maximal absorption wavelength λ1 of the first compound and the maximal absorption wavelength λ2 of the second compound preferably satisfy a relationship of the expression (X-1), and more preferable to satisfy a relationship of the expression (X-2).

$$- 10 nm \leq λ 1 - λ 2 \leq 10 nm \qquad Expression ( X - 1 )$$

$$- 5 nm \leq λ 1 - λ 2 \leq 5 nm \qquad Expression ( X - 2 )$$

More specifically, the second compound is preferably a compound selected from the group consisting of a compound having an imidazoline skeleton, a pyrromethene boron complex, a subphthalocyanine compound, a squarylium compound, and a compound having a triarylamine skeleton; more preferably a compound having an imidazoline skeleton; and still more preferably a compound represented by Formula (11) described later.

### Compound Having Imidazoline Skeleton

Examples of the compound having an imidazoline skeleton include a compound represented by Formula (11).

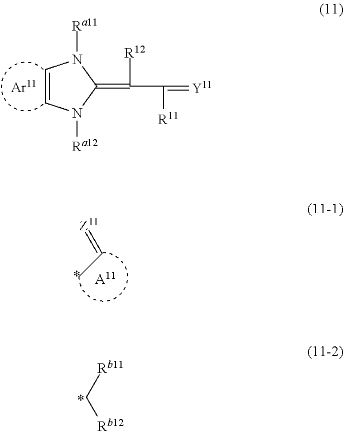

In Formula (11), Y¹ represents a group represented by Formula (11-1) or a group represented by Formula (11-2). Among these, from the viewpoint that the effect of the present invention is more excellent, a group represented by Formula (11-1) is preferable. In Formulae (11-1) and (11-2), * represents a bonding position, and a carbon atom marked with * and a carbon atom bonded to R¹¹ form a double bond.

In Formula (11-1), Z¹¹ represents an oxygen atom, a sulfur atom, ═NR^{Z11}, or ═CR^{Z12}R^{Z13}.

R^{Z11} represents a hydrogen atom or a substituent. R^{Z12} and R^{Z13} each independently represent a cyano group or —COOR^{Z14}. R^{Z14} represents an alkyl group which may have a substituent, an aryl group which may have a substituent, or a heteroaryl group which may have a substituent.

Z¹¹ is preferably an oxygen atom.

In Formula (11-1), A¹¹ represents a ring which contains at least two carbon atoms and may have a substituent. The two carbon atoms mean a carbon atom which is bonded to Z¹¹ specified in Formula (11-1) and a carbon atom which is adjacent to the carbon atom bonded to Z¹¹ and is specified in Formula (11-1) (a carbon atom forming the double bond with the carbon atom bonded to R¹¹), and any of the two carbon atoms is an atom constituting A¹¹.

Specific aspects and suitable aspects of the ring which may have a substituent, represented by A¹¹, are the same as the specific aspect and the suitable aspect of the ring which may have a substituent, represented by A¹.

In Formula (11-2), R^{b11} and R^{b12} each independently represent a cyano group or —COOR^{b13}.

R^{b13} represents an alkyl group which may have a substituent, an aryl group (phenyl group or the like) which may have a substituent, or a heteroaryl group which may have a substituent.

In Formula (11), R¹¹ and R¹² each independently represent a hydrogen atom or a substituent. R¹¹ and R¹² are preferably a hydrogen atom.

R^{a11} and R^{a12} each independently represent an aryl group which may have a substituent, —C(R^{L11})(R^{L12})(R^{L13}), or a heteroaryl group which may have a substituent. Here, R^{a11} and R^{a12} represent groups different from each other.

R^{L11} to R^{L13} in —C(R^{L11})(R^{L12})(R^{L13}) each independently represent an alkyl group which may have a substituent, an aryl group which may have a substituent, a heteroaryl group which may have a substituent, or a hydrogen atom, and two or more of R^{L11} to R^{L13} each independently represent an alkyl group which may have a substituent, an aryl group which may have a substituent, or a heteroaryl group which may have a substituent.

The alkyl group which may have a substituent, the aryl group which may have a substituent, and the heteroaryl group which may have a substituent, which are represented by R^{L11} to R^{L13}, may be bonded to each other to form a ring.

Specific aspects and suitable aspects of R^{a11}, R^{a12}, and R^{L11} to R^{L13} in Formula (11) are the same as the specific aspects and the suitable aspects of R^{a1}, R^{a2}, and R^{L1} to R^{L3} in Formula (1).

In Formula (11), Ar¹¹ represents an aromatic ring which may have a substituent.

Specific aspects and suitable aspects of Ar¹¹ are the same as the specific aspects and the suitable aspects of Ar¹ in Formula (1).

The compound represented by Formula (11) is preferably the above-described compound represented by Formula (2), and more preferably the above-described compound represented by Formula (3). In addition, the compound represented by Formula (11) may be the compound represented by Formula (4) or the compound represented by Formula (5).

Specific examples of the compound represented by Formula (11) include each compound exemplified as the compound represented by Formula (1) described above.

### Pyrromethene Boron Complex

Examples of the pyrromethene boron complex include a compound represented by Formula (P-1).

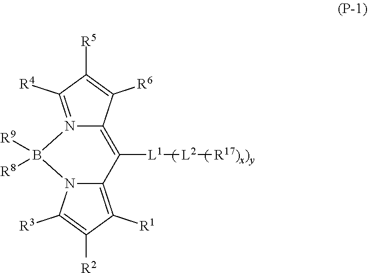

R¹ to R⁶ each independently represent a hydrogen atom or a substituent.

Examples of the substituent include an alkyl group which may have a substituent, an aliphatic heterocyclic group which may have a substituent, an alkenyl group which may have a substituent, an alkynyl group which may have a substituent, a hydroxyl group, a thiol group, an alkoxy group which may have a substituent, an alkylthio group which may have a substituent, an aryl ether group which may have a substituent, an aryl thioether group which may have a substituent, an aryl group which may have a substituent, a heteroaryl group which may have a substituent, a halogen atom, a cyano group, an aldehyde group, a carbonyl group which may have a substituent, a carboxyl group which may have a substituent, an oxycarbonyl group which may have a substituent, a carbamoyl group which may have a substituent, an alkyloxycarbonyl group which may have a substituent, an amino group which may have a substituent, a nitro group, a silyl group which may have a substituent, a siloxanyl group which may have a substituent, a boryl group which may have a substituent, and a phosphine oxide group which may have a substituent.

R⁸ and R⁹ each independently represent an alkyl group which may have a substituent, an aliphatic heterocyclic group which may have a substituent, an alkenyl group which may have a substituent, an alkynyl group which may have a substituent, a hydroxyl group, a thiol group, an alkoxy group which may have a substituent, an alkylthio group which may have a substituent, an aryl ether group which may have a substituent, an aryl thioether group which may have a substituent, an aryl group which may have a substituent, a heteroaryl group which may have a substituent, or a halogen atom.

L¹ represents a single bond or a (y+1)-valent linking group, and is preferably a single bond, an arylene group which may have a substituent, or a heteroarylene group which may have a substituent.

L² represents a single bond, an (x+1)-valent aromatic hydrocarbon ring group, or an (x+1)-valent aromatic heterocyclic group.

x and y each independently represent an integer of 1 to 5.

R¹⁷ represents an electron-withdrawing group.

In addition, in all the above-described groups, as a substituent in a case of being substituted, an alkyl group, an aliphatic heterocyclic group, an alkenyl group, an alkynyl group, a hydroxyl group, a thiol group, an alkoxy group, an alkylthio group, an aryl ether group, an aryl thioether group, an aryl group, a heteroaryl group, a halogen atom, a cyano group, an aldehyde group, a carbonyl group, a carboxyl group, an oxycarbonyl group, a carbamoyl group, an amino group, a nitro group, a silyl group, a siloxanyl group, a boryl group, or a phosphine oxide group is preferable; and furthermore, a specific substituent which is preferred in the description of each substituent is preferable. In addition, these substituents may be further substituted with the above-described substituent.

The alkyl group may be linear, branched, or cyclic.

The number of carbon atoms in the alkyl group is not particularly limited, but from the viewpoint of ease of availability and cost, it is preferably 1 to 20 and more preferably 1 to 8.

Examples of the aryl group include a phenyl group, a biphenyl group, a terphenyl group, a naphthyl group, a fluorenyl group, a phenanthryl group, an anthracenyl group, a pyrenyl group, a fluoranthenyl group, and a triphenylenyl group.

The number of carbon atoms in the aryl group is not particularly limited, but is preferably 6 to 40 and more preferably 6 to 30.

In a case where R¹ to R⁶, R⁸, and R⁹ are an aryl group which may have a substituent, as the aryl group, a phenyl group, a biphenyl group, a terphenyl group, a naphthyl group, a fluorenyl group, a phenanthryl group, or an anthracenyl group is preferable; a phenyl group, a biphenyl group, a terphenyl group, or a naphthyl group is more preferable; a phenyl group, a biphenyl group, or a terphenyl group is still more preferable; and a phenyl group is particularly preferable.

The halogen atom refers to an atom selected from a fluorine atom, a chlorine atom, a bromine atom, and an iodine atom.

Preferred examples of the electron-withdrawing group include a fluorine atom, a fluorine-containing aryl group, a fluorine-containing heteroaryl group, a fluorine-containing alkyl group, an acyl group which may have a substituent, an alkyloxycarbonyl group which may have a substituent, and a cyano group.

From the viewpoint of photostability, L¹ is preferably a single bond or an arylene group which may have a substituent.

L² is preferably an (x+1)-valent aromatic hydrocarbon ring.

Examples of a ring constituting the (x+1)-valent aromatic hydrocarbon ring represented by L² include a benzene ring, a naphthalene ring, and an anthracene ring.

Examples of a ring constituting the (x+1)-valent aromatic heterocyclic ring represented by L² include known rings.

In a case where R¹⁷ is fluorine, x and y are preferably 1 to 5. In a case where R¹⁷ is fluorine, since the fluorine consists of one atom, the decrease in fluorescence quantum yield due to molecular vibration hardly occurs in the first place, and the effect of improving durability is larger, so that in a case where R¹⁷ is fluorine, x and y are preferably 1 to 5.

In addition, one particularly preferred example of the compound represented by Formula (P-1) includes a case in which all of R¹, R³, R⁴, and R⁶ are alkyl groups which may have a substituent, all the alkyl groups may be the same or different from each other, L¹ is a single bond, L² is an aromatic hydrocarbon ring which may have a substituent, x is 5, and y is 1.

Examples of the pyrromethene boron complex represented by Formula (P-1) are shown below, but the present invention is not limited thereto. In addition, examples of the compound represented by Formula (P-1) also include compounds described in paragraphs [0136] to [0149] of WO2016/190283A, the contents of which are incorporated herein by reference.

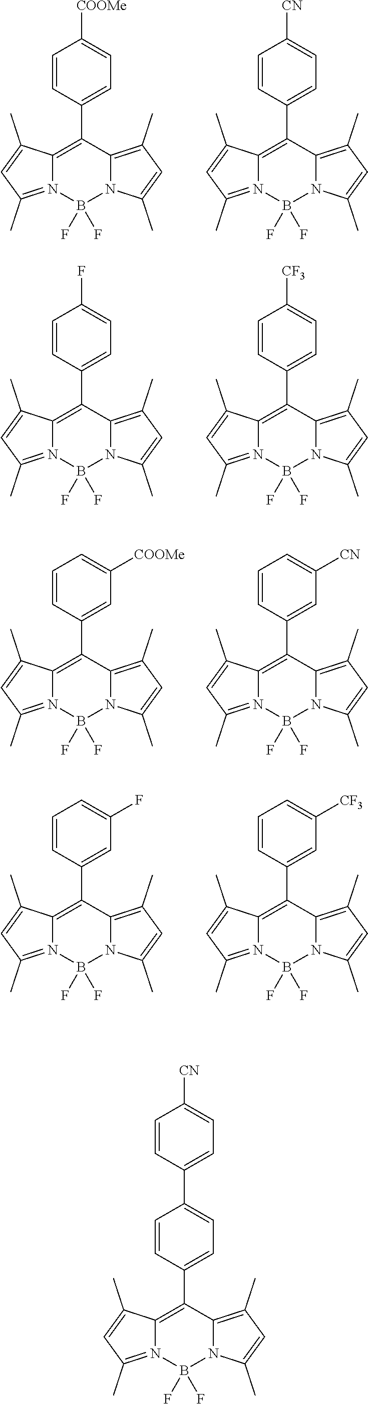

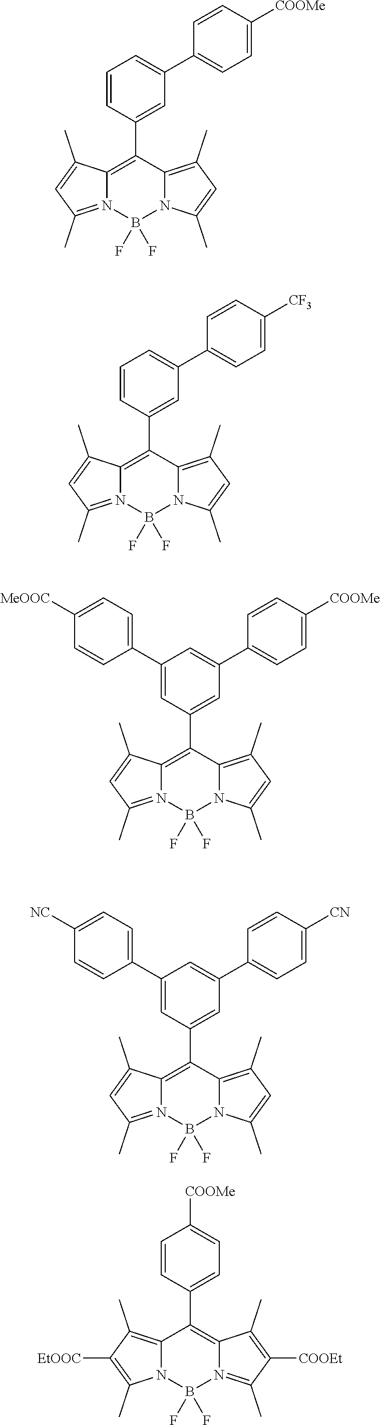

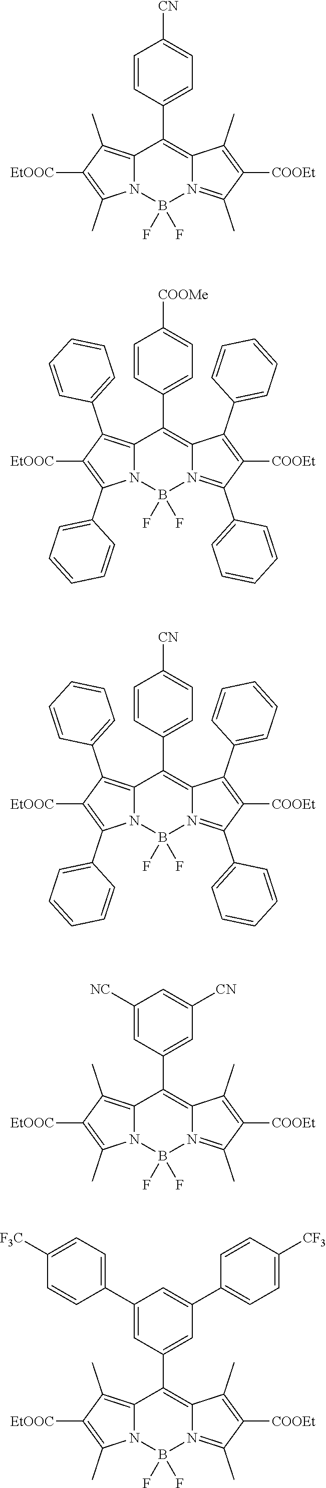

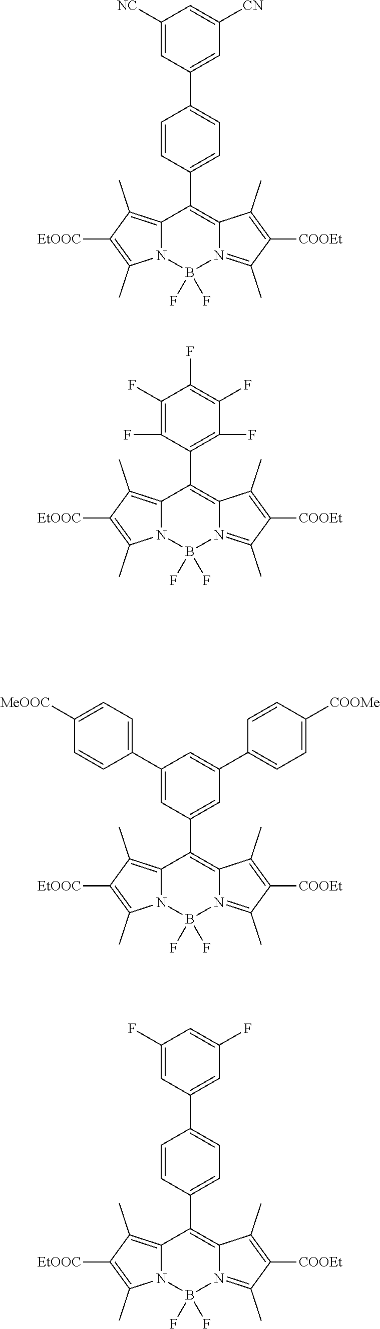

### Subphthalocyanine Compound

Examples of the subphthalocyanine compound include a compound represented by Formula (SP-1).

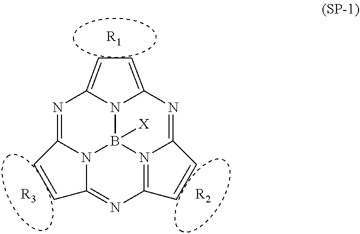

In Formula (SP-1), X represents a halogen atom (preferably a fluorine atom or a chlorine atom), a hydroxy group, a thiol group, an amino group, an alkoxy group which may have a substituent, an aryloxy group which may have a substituent, an alkyl group which may have a substituent, an alkylamine group which may have a substituent, an arylamine group which may have a substituent, an alkylthio group which may have a substituent, or an arylthio group which may have a substituent.

R₁ to R₃ each independently represent a ring structure which may have a substituent. It is preferable that at least one of R₁ to R₃ contains at least one heteroatom in the ring structure.

In Formula (SP-1), one of bonds between boron and nitrogen at the center is a coordinate bond.

In Formula (SP-1), in a case where the ring structure of R₁ to R₃ contains at least one or more heteroatoms, the compound can have suitable light absorption characteristics as a photoelectric conversion film which absorbs green light. Specifically, in a case where the ring structure of R₁ to R₃ contains at least one or more heteroatoms, the compound has light absorption characteristics capable of reducing absorption of light in a long wavelength range and selectively absorbing light in a green light range.

In addition, in Formula (SP-1), it is preferable that at least one of R₁ to R₃ is a ring structure having a substituent. In particular, in a case where at least one of R₁ to R₃ is a ring structure substituted with an electron-withdrawing group, the compound represented by Formula (SP-1) can be synthesized with a higher yield, which is preferable. For example, in Formula (SP-1), at least one of R₁ to R₃ may have a ring structure having a halogen atom as a substituent.

Here, in Formula (SP-1), R₁ to R₃ may be a ring structure in which a part of hydrogen atoms is substituted with a substituent, or may be a ring structure in which all hydrogen atoms are substituted with a substituent. In addition, the substituent may be substituted into the ring structure of R₁ to R₃ such that the compound represented by Formula (SP-1) has symmetry, or may be substituted into the ring structure of R₁ to R₃ such that the compound represented by Formula (SP-1) does not have symmetry.

In addition, in Formula (SP-1), it is preferable that R₁ to R₃ represent a ring structure having a 7-conjugated system structure. In a case where R₁ to R₃ are ring structures having a 7-conjugated system structure, the compound represented by Formula (SP-1) can have a suitable absorption spectrum for absorbing green light having a wavelength of 490 to 600 nm.

From the viewpoint of reducing absorption of blue light, which is in a shorter wavelength range than the green light, in the photoelectric conversion film, the ring structure represented by R₁ to R₃ is preferably an aromatic ring structure. Examples of the aromatic ring constituting the aromatic ring structure include an aromatic hydrocarbon ring and an aromatic heterocyclic ring.

In addition, in Formula (SP-1), R₁ to R₃ may have a ring structure having an arbitrary number of ring-constituting atoms. Furthermore, R₁ to R₃ may have a monocyclic structure or a fused-ring structure. Here, R₁ to R₃ are preferably a ring structure having 3 to 8 ring-constituting atoms, and more preferably a ring structure having 6 ring-constituting atoms.

Furthermore, the heteroatom contained in the ring structure of R₁ to R₃ is preferably a nitrogen atom. In a case where the ring structure of R₁ to R₃ contains a nitrogen atom, the compound represented by Formula (SP-1) is suitable for use in a photoelectric conversion film which absorbs green light, since the absorption region is shifted to the short wavelength side and the absorption of light in a long wavelength region is reduced.

Examples of the ring structure containing a nitrogen atom include a pyridine ring, a pyridazine ring, a pyrimidine ring, a pyrazine ring, a tetrazine ring, a pyrrole ring, and an imidazole ring.

The heteroatom contained in the ring structure of R₁ to R₃ may be contained in the ring structure of R₁ to R₃ such that the compound represented by Formula (SP-1) has symmetry, or may be contained in the ring structure of R₁ to R₃ such that the compound represented by Formula (SP-1) does not have symmetry.

Here, specific examples of the ring structure of the subphthalocyanine compound are shown in the following structural examples (1) to (17). However, the ring structure of the subphthalocyanine compound according to one embodiment of the present disclosure is not limited to the following structural examples (1) to (17).

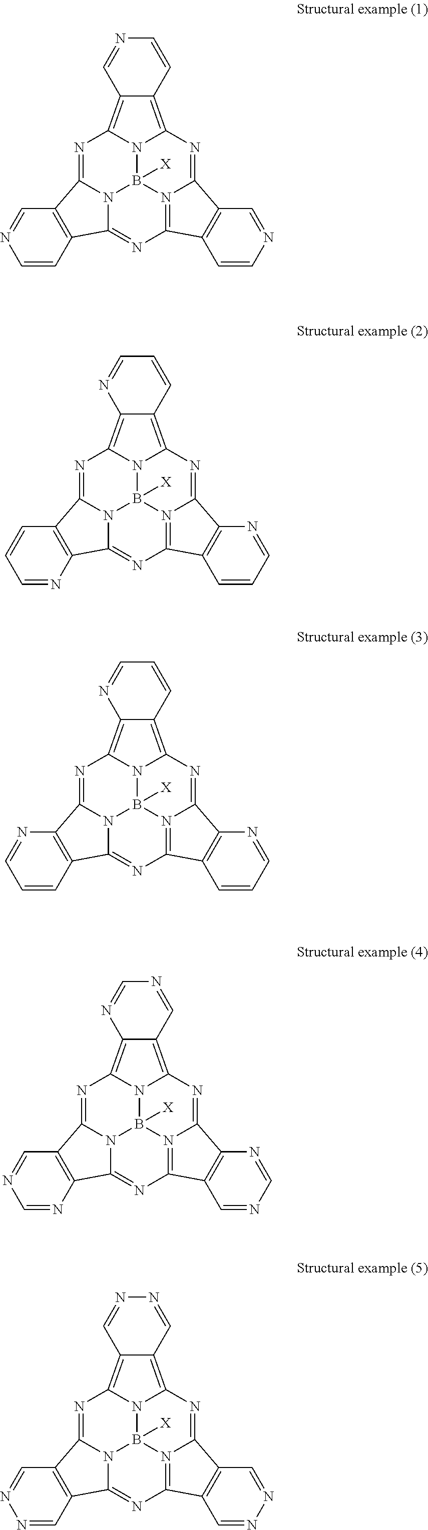

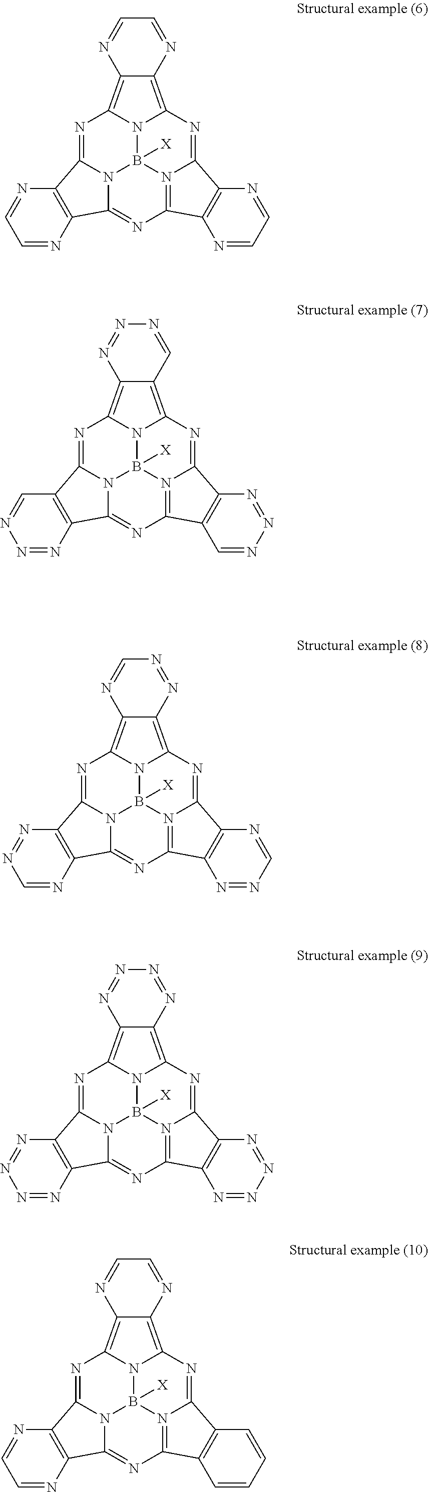

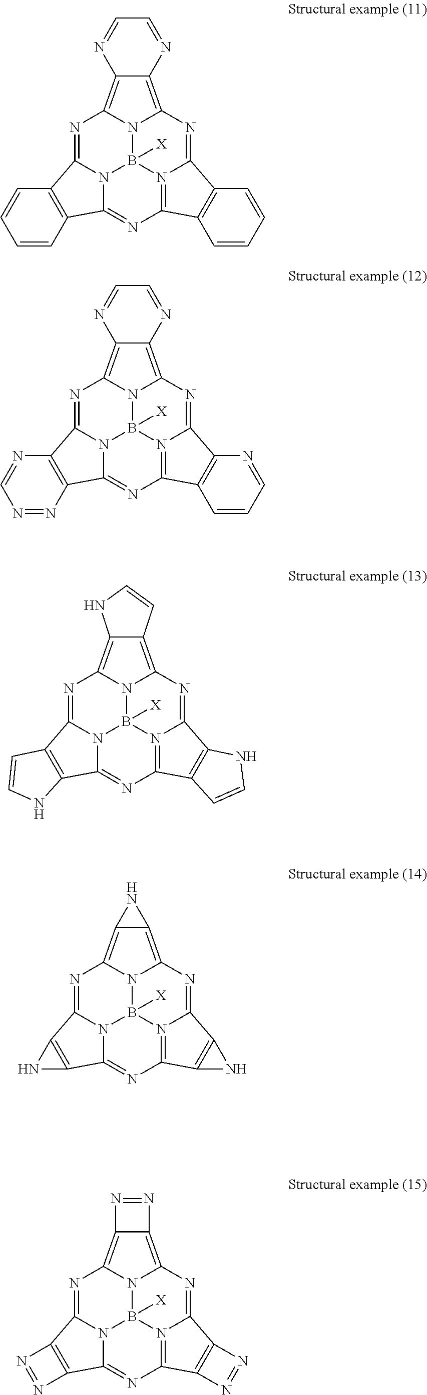

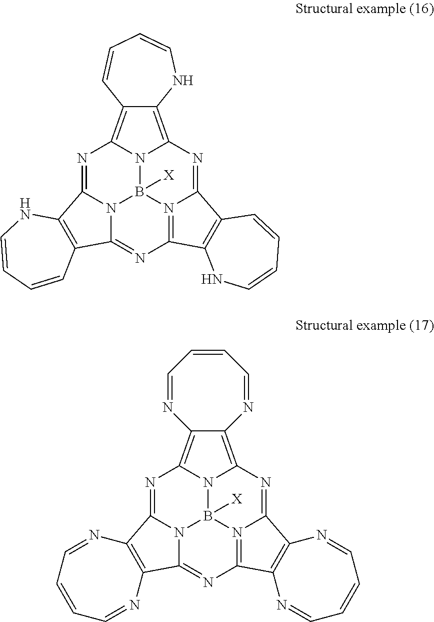

In the above-described structural examples (1) to (17), X is as described above.

### Squarylium Compound

Examples of the squarylium compound include a compound represented by Formula (S-1).

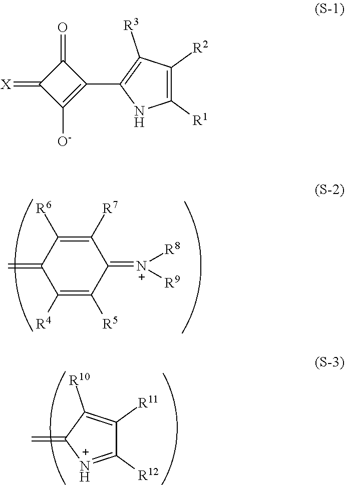

In Formula (S-1), R¹ to R³ each independently represent a hydrogen atom, an alkyl group having 1 to 6 carbon atoms, or an acetyl group.

The alkyl group having 1 to 6 carbon atoms, represented by R¹ to R³, is not limited to any of linear, branched, or cyclic forms as long as it is an alkyl group having 1 to 6 carbon atoms; and specific examples thereof include a methyl group, an ethyl group, a n-propyl group, an i-propyl group, a n-butyl group, an i-butyl group, a sec-butyl group, a tert-butyl group, a n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a n-hexyl group, a 1-methylpentyl group, a 2-methylpentyl group, a 3-methylpentyl group, a 4-methylpentyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1-ethyl-1-methylpropyl group, and a cyclohexyl group.

As R¹ to R³ in Formula (S-1), it is preferable that R¹ and R³ are each an alkyl group having 1 to 6 carbon atoms and R² is a hydrogen atom, an alkyl group having 1 to 6 carbon atoms, or an acetyl group; it is more preferable that R¹ and R³ are each a linear alkyl group having 1 to 6 carbon atoms and R² is a hydrogen atom, a linear alkyl group having 1 to 6 carbon atoms, or an acetyl group; it is still more preferable that R¹ and R³ are each the same linear alkyl group having 1 to 6 carbon atoms and R² is a hydrogen atom, a linear alkyl group having 1 or 6 carbon atoms, or an acetyl group; and it is particularly preferable that R¹ and R³ are each a methyl group and R² is a hydrogen atom, a linear alkyl group having 1 or 2 carbon atoms, or an acetyl group.

In Formula (S-1), X represents a group represented by Formula (S-2) or Formula (S-3), and is preferably a group represented by Formula (S-3).

In Formula (S-2), R⁴ to R⁷ each independently represent a hydrogen atom, a fluorine atom, a chlorine atom, a methyl group, a methoxy group, or a hydroxy group; and it is preferable that one of R⁴ to R⁷ is a fluorine atom, a methyl group, a methoxy group, or a hydroxy group, and the remaining three are hydrogen atoms, it is more preferable that one of R⁴ or R⁶ is a fluorine atom, a methyl group, a methoxy group, or a hydroxy group, and the remaining three are hydrogen atoms, and it is still more preferable that one of R⁴ or R⁶ is a hydroxy group, and the remaining three are hydrogen atoms.

In Formula (S-2), R⁸ and R⁹ each independently represent an alkyl group having 1 to 6 carbon atoms.

Specific examples of the alkyl group having 1 to 6 carbon atoms, represented by R⁸ and R⁹ in Formula (S-2), include the same one as the specific examples of the alkyl group having 1 to 6 carbon atoms, represented by R¹ to R³ in Formula (S-1).

R⁸ and R⁹ in Formula (S-2) are each preferably a linear alkyl group having 1 to 6 carbon atoms, and more preferably a linear alkyl group having 1 to 4 carbon atoms.

In Formula (S-3), R¹⁰ to R¹² each independently represent a hydrogen atom, an alkyl group having 1 to 6 carbon atoms, or an acetyl group.

Specific examples of the alkyl group having 1 to 6 carbon atoms, represented by R¹⁰ to R¹² in Formula (S-3), include the same one as the specific examples of the alkyl group having 1 to 6 carbon atoms, represented by R¹ to R³ in Formula (S-1).

As R¹⁰ to R¹² in Formula (S-3), it is preferable that R¹⁰ and R¹² are each an alkyl group having 1 to 6 carbon atoms and R¹¹ is a hydrogen atom, an alkyl group having 1 to 6 carbon atoms, or an acetyl group; it is more preferable that R¹⁰ and R¹² are each a linear alkyl group having 1 to 6 carbon atoms and R¹¹ is a hydrogen atom, a linear alkyl group having 1 to 6 carbon atoms, or an acetyl group; it is still more preferable that R¹⁰ and R¹² are each the same linear alkyl group having 1 to 6 carbon atoms and R¹¹ is a hydrogen atom, a linear alkyl group having 1 or 6 carbon atoms, or an acetyl group; and it is particularly preferable that R¹⁰ and R¹² are each a methyl group and R¹¹ is a hydrogen atom, a linear alkyl group having 1 or 2 carbon atoms, or an acetyl group.

As the compound represented by Formula (S-1), a combination of preferred ones of R¹ to R¹² and X is more preferable, and a combination of more preferred ones thereof is still more preferable.

Specific examples of the compound represented by Formula (S-1) are shown below, but the present invention is not limited to these specific examples.

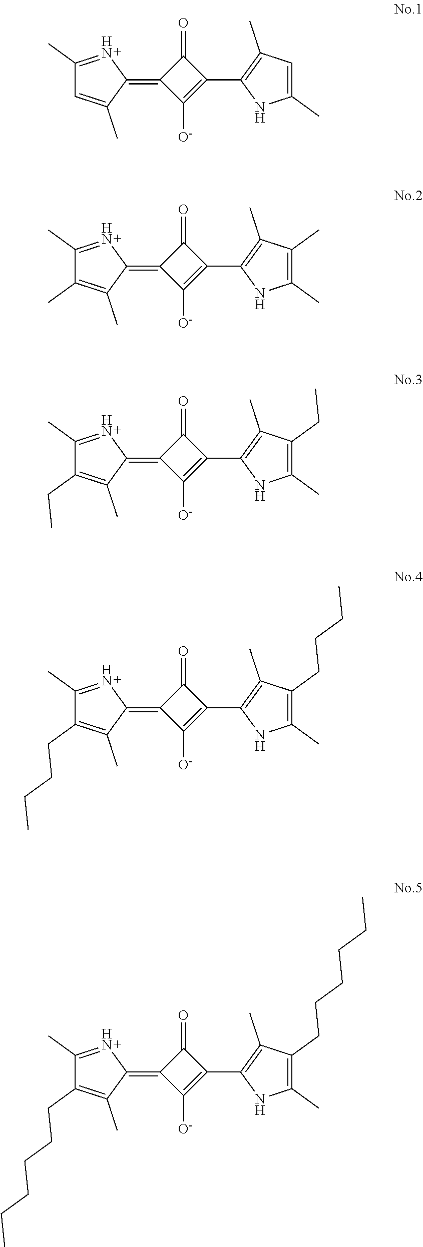

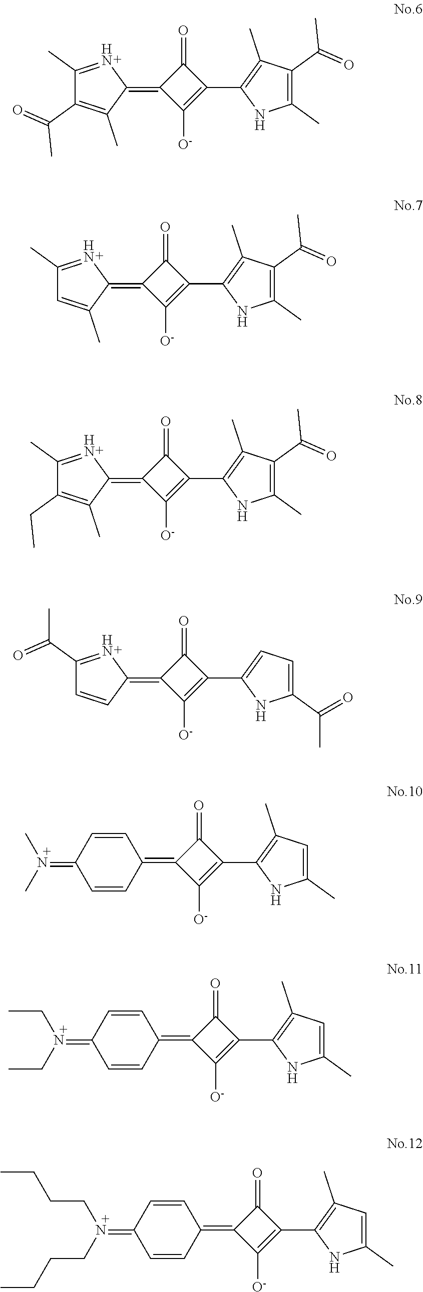

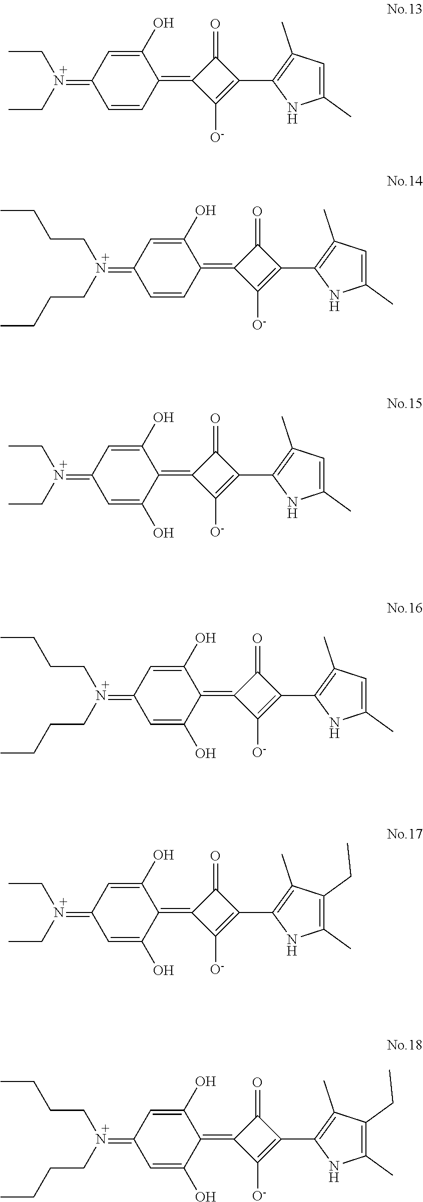

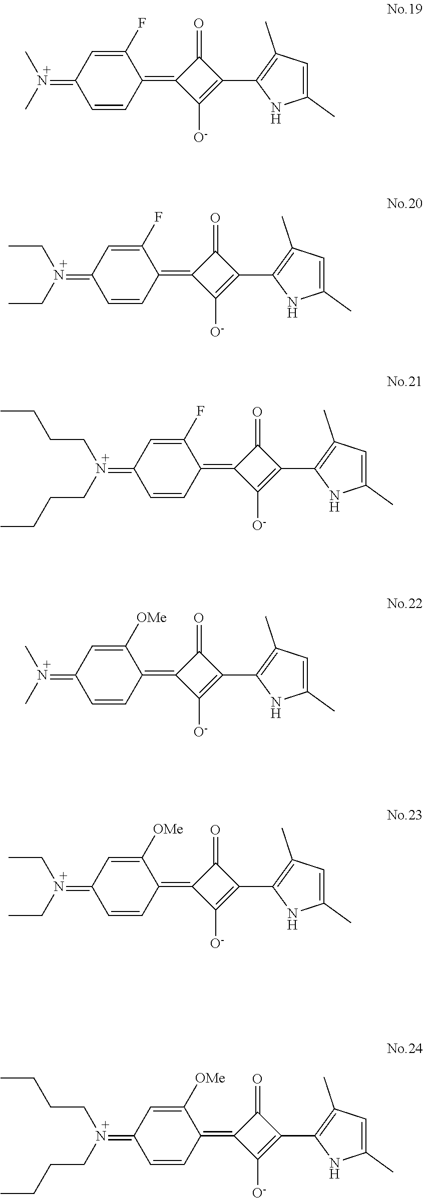

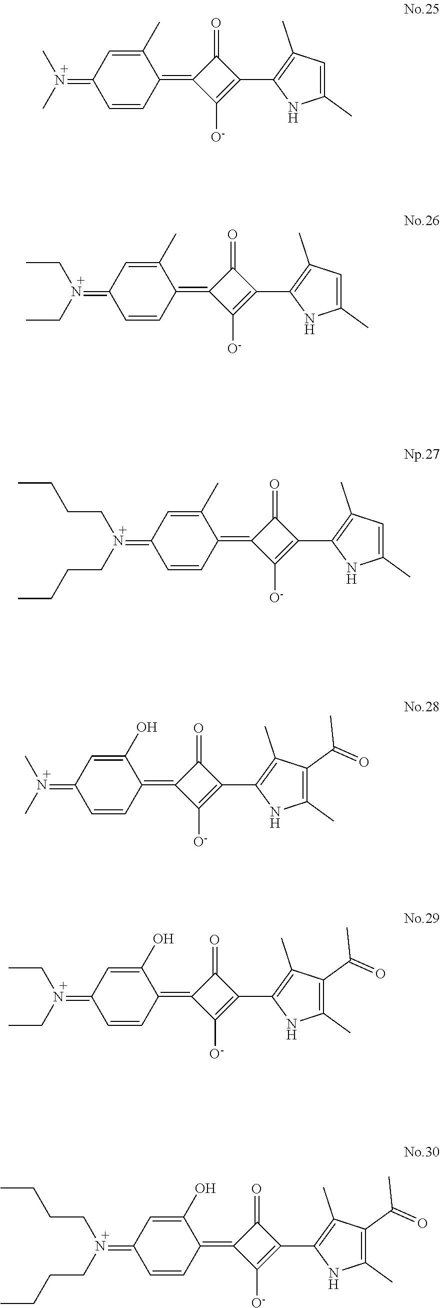

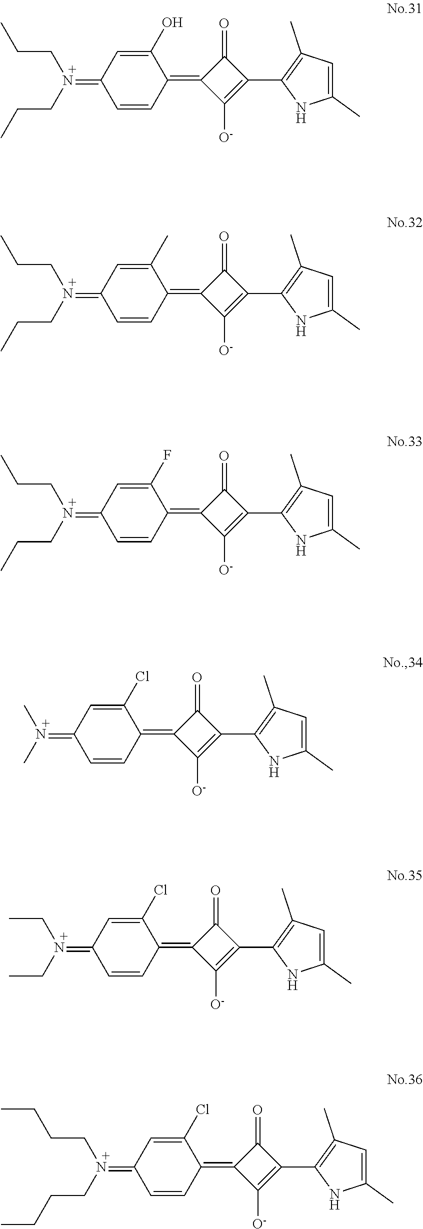

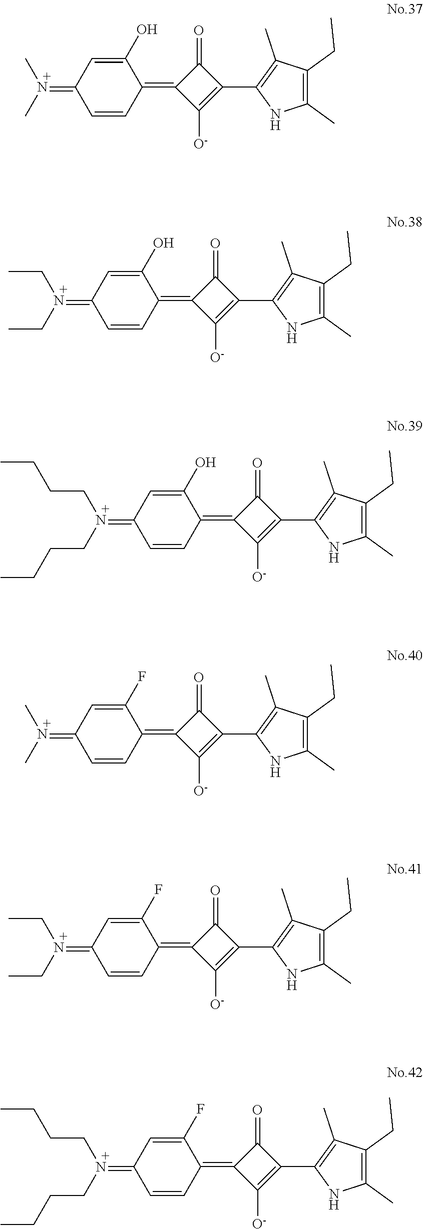

### Compound Having Triarylamine Skeleton

Examples of the compound having a triarylamine skeleton include a compound represented by Formula (T) and a compound represented by Formula (U). As the compound having a triarylamine skeleton, a compound represented by Formula (U) is preferable.

### Compound Represented by Formula (T)

Formula (T) is shown below.

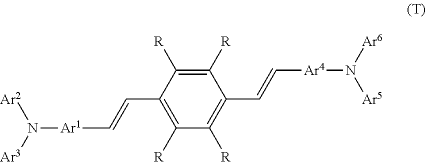

In Formula (T), Ar¹ and Ar⁴ each independently represent an arylene group which may have a substituent or a heteroarylene group which may have a substituent.

Ar², Ar³, Ar⁵, and Ar⁶ each independently represent an aryl group which may have a substituent or a heteroaryl group which may have a substituent.

Four R's each independently represent a hydrogen atom or a substituent (preferably a cyano group).

Specific examples of the compound represented by Formula (T) are shown below, but the present invention is not limited to these specific examples.

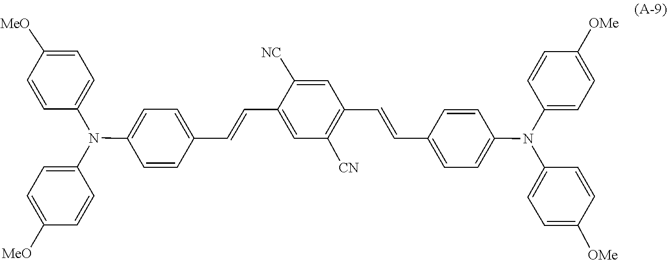 [SMILES: COc1ccc(N(c2ccc(/C=C/c3cc(C#N)c(/C=C/c4ccc(N(c5ccc(OC)cc5)c5ccc(OC)cc5)cc4)cc3C#N)cc2)c2ccc(OC)cc2)cc1]

### Compound Represented by Formula (U)

Formula (U) is shown below.

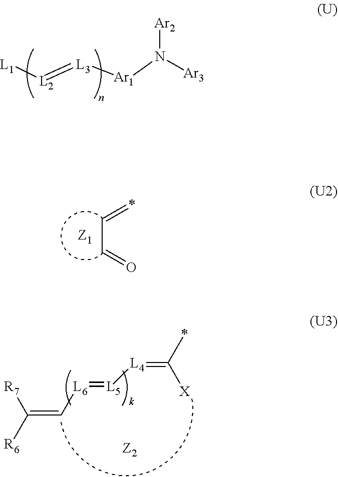

In Formula (U), L₂ and L₃ each independently represent a methine group which may have a substituent.

n represents an integer of 0 to 2.

Ar₁ represents an arylene group which may have a substituent or a heteroarylene group which may have a substituent.

Ar₂ and Ar₃ each independently represent an aryl group which may have a substituent, an alkyl group which may have a substituent, or a heteroaryl group which may have a substituent.

Ar₁ and Ar₂, Ar₂ and Ar₃, or Ar₃ and Ar₁ (preferably, a substituent in an arylene group or a heteroarylene group in Ar₁ and a substituent in an aryl group or a heteroaryl group in Ar₂, a substituent in an aryl group or a heteroaryl group in Ar₂ and a substituent in an aryl group or a heteroaryl group in Ar₃, and a substituent in an arylene group or a heteroarylene group in Ar₁ and a substituent in an aryl group or a heteroaryl group in Ar₂) may be bonded to each other to form a ring.

L₁ represents a methine group which may have a substituent and is bonded to a group represented by Formula (U2), or a group represented by Formula (U3).

In Formula (U2), Z₁ represents a 5-membered ring which is a ring including a carbon atom bonded to L₁ and a carbonyl group adjacent to the carbon atom and may have a substituent, a 6-membered ring which may have a substituent, or a fused ring which includes at least one of a 5-membered ring or a 6-membered ring and may have a substituent. Such a ring is preferably a ring which is generally used as an acidic nucleus in a merocyanine dye.

* represents a bonding position with a methine group which may have a substituent, represented by L₁.

In Formula (U3), X represents a heteroatom.

Z₂ represents a ring containing X, which may be a 5-membered ring, a 6-membered ring, or a 7-membered ring, each of which may have a substituent, or a fused ring which includes at least one of a 5-membered ring, a 6-membered ring, or a 7-membered ring and may have a substituent.

L₄ to L₆ each independently represent a methine group which may have a substituent.

In a case of a plurality of L₅'s and/or a plurality of L₆'s, the plurality of L₅'s and/or the plurality of L₆'s may be the same or different from each other.

R₆ and R₇ each independently represent a hydrogen atom or a substituent. R₆ and R₇ may be bonded to each other to form a ring.

k represents an integer of 0 to 2.

* represents a bonding position to which L₂ or Ar₁ is bonded.

As the compound represented by Formula (U), the compound represented by General Formula (1) in JP2015-118977A can also be used, the contents of which are incorporated herein by reference.

Specific examples of the compound represented by Formula (U) are shown below, but the present invention is not limited to these specific examples.

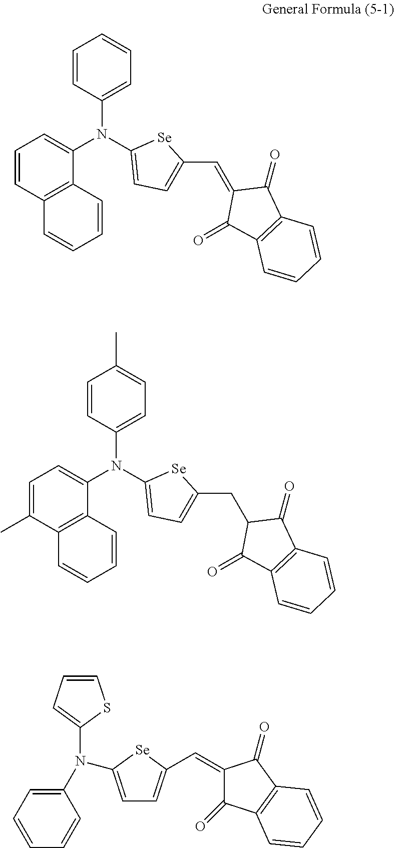 [SMILES: O=C1C(=Cc2ccc(N(c3ccccc3)c3cccc4ccccc34)[se]2)C(=O)c2ccccc21 ; Cc1ccc(N(c2ccc(CC3C(=O)c4ccccc4C3=O)[se]2)c2ccc(C)c3ccccc23)cc1 ; O=C1C(=Cc2ccc(N(c3ccccc3)c3cccs3)[se]2)C(=O)c2ccccc21]

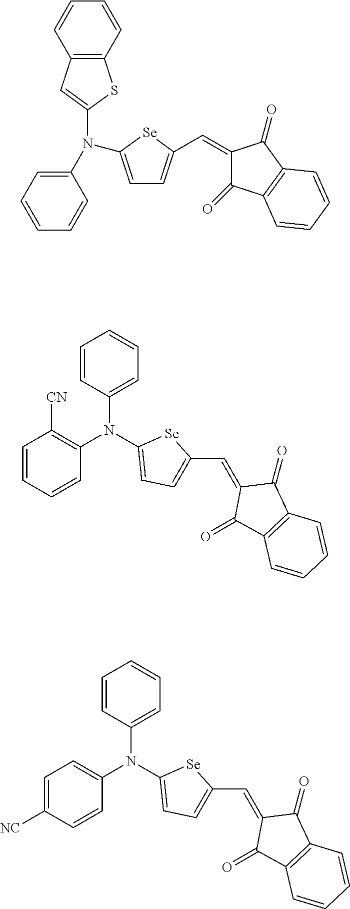 [SMILES: O=C1C(=Cc2ccc(N(c3ccccc3)c3cc4ccccc4s3)[se]2)C(=O)c2ccccc21 ; N#Cc1ccccc1N(c1ccccc1)c1ccc(C=C2C(=O)c3ccccc3C2=O)[se]1 ; N#Cc1ccc(N(c2ccccc2)c2ccc(C=C3C(=O)c4ccccc4C3=O)[se]2)cc1]

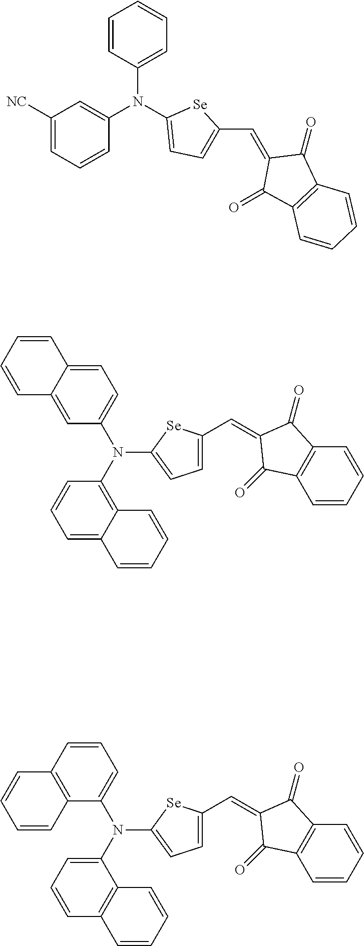 [SMILES: N#Cc1cccc(N(c2ccccc2)c2ccc(C=C3C(=O)c4ccccc4C3=O)[se]2)c1 ; O=C1C(=Cc2ccc(N(c3ccc4ccccc4c3)c3cccc4ccccc34)[se]2)C(=O)c2ccccc21 ; O=C1C(=Cc2ccc(N(c3cccc4ccccc34)c3cccc4ccccc34)[se]2)C(=O)c2ccccc21]

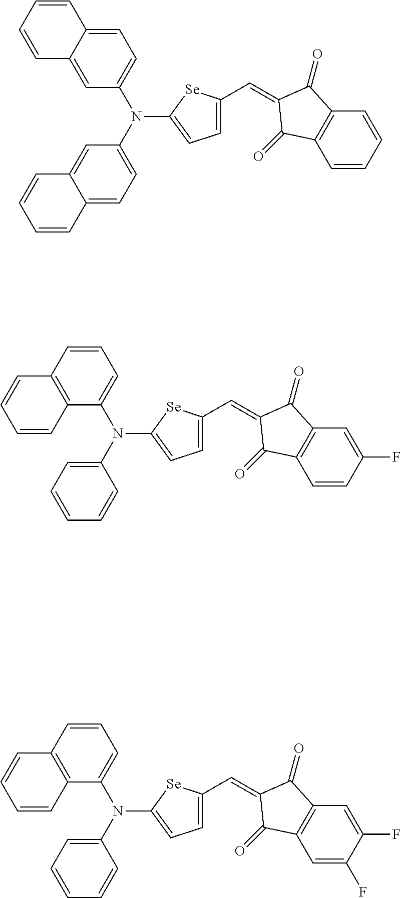 [SMILES: O=C1C(=Cc2ccc(N(c3ccc4ccccc4c3)c3ccc4ccccc4c3)[se]2)C(=O)c2ccccc21 ; O=C1/C(=C\c2ccc(N(c3ccccc3)c3cccc4ccccc34)[se]2)C(=O)c2cc(F)ccc21 ; O=C1C(=Cc2ccc(N(c3ccccc3)c3cccc4ccccc34)[se]2)C(=O)c2cc(F)c(F)cc21]

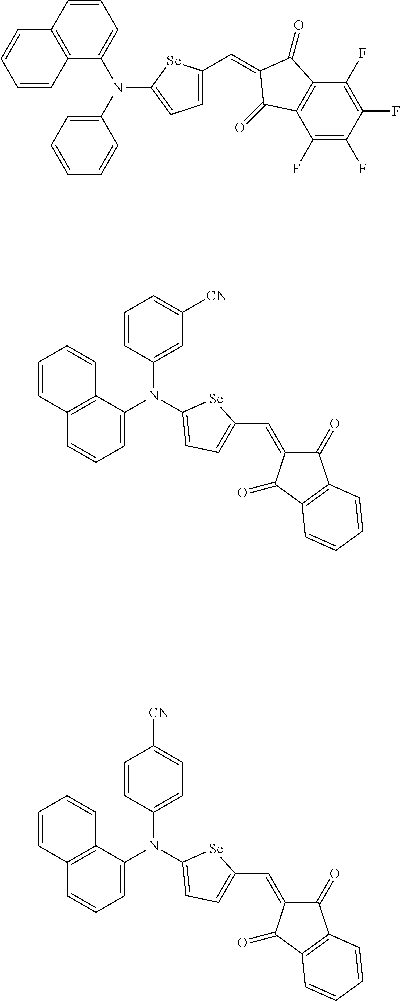 [SMILES: O=C1C(=Cc2ccc(N(c3ccccc3)c3cccc4ccccc34)[se]2)C(=O)c2c(F)c(F)c(F)c(F)c21 ; N#Cc1cccc(N(c2ccc(C=C3C(=O)c4ccccc4C3=O)[se]2)c2cccc3ccccc23)c1 ; N#Cc1ccc(N(c2ccc(C=C3C(=O)c4ccccc4C3=O)[se]2)c2cccc3ccccc23)cc1]

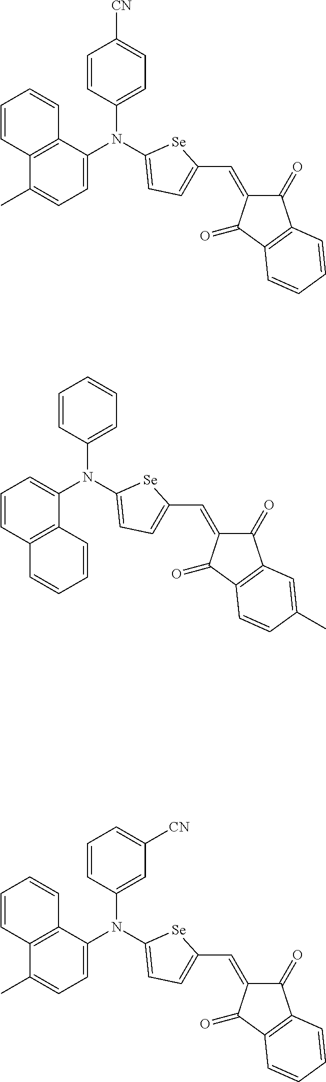 [SMILES: Cc1ccc(N(c2ccc(C#N)cc2)c2ccc(C=C3C(=O)c4ccccc4C3=O)[se]2)c2ccccc12 ; Cc1ccc2c(c1)C(=O)/C(=C/c1ccc(N(c3ccccc3)c3cccc4ccccc34)[se]1)C2=O ; Cc1ccc(N(c2cccc(C#N)c2)c2ccc(C=C3C(=O)c4ccccc4C3=O)[se]2)c2ccccc12]

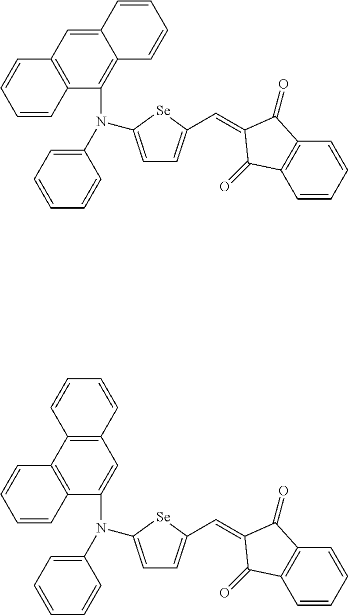 [SMILES: O=C1C(=Cc2ccc(N(c3ccccc3)c3c4ccccc4cc4ccccc34)[se]2)C(=O)c2ccccc21 ; O=C1C(=Cc2ccc(N(c3ccccc3)c3cc4ccccc4c4ccccc34)[se]2)C(=O)c2ccccc21]

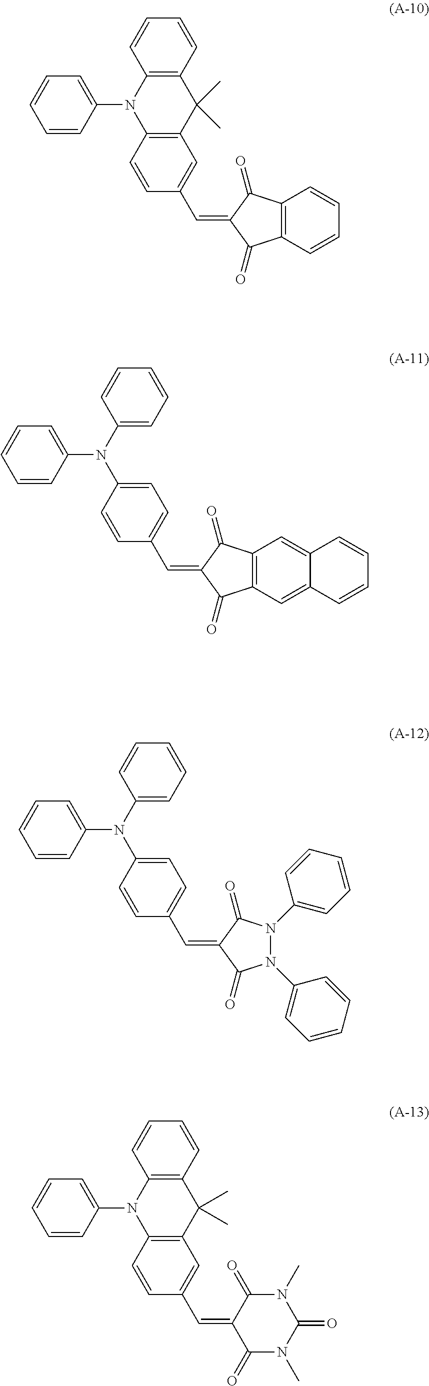 [SMILES: CC1(C)c2ccccc2N(c2ccccc2)c2ccc(C=C3C(=O)c4ccccc4C3=O)cc21 ; O=C1C(=Cc2ccc(N(c3ccccc3)c3ccccc3)cc2)C(=O)c2cc3ccccc3cc21 ; O=C1C(=Cc2ccc(N(c3ccccc3)c3ccccc3)cc2)C(=O)N(c2ccccc2)N1c1ccccc1 ; CN1C(=O)C(=Cc2ccc3c(c2)C(C)(C)c2ccccc2N3c2ccccc2)C(=O)N(C)C1=O]

In addition to the above, compounds represented by General Formula (5-1), General Formula (5-2), General Formula (5-3), General Formula (5-4), or General Formula (5-5) shown below can also be exemplified as the specific examples of the compound having a triarylamine skeleton, represented by Formula (U).

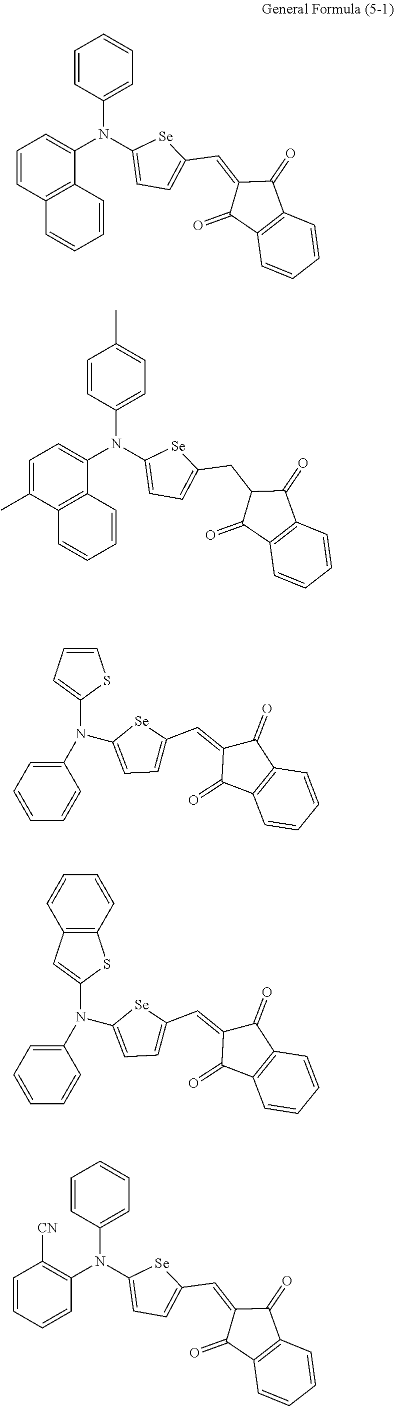

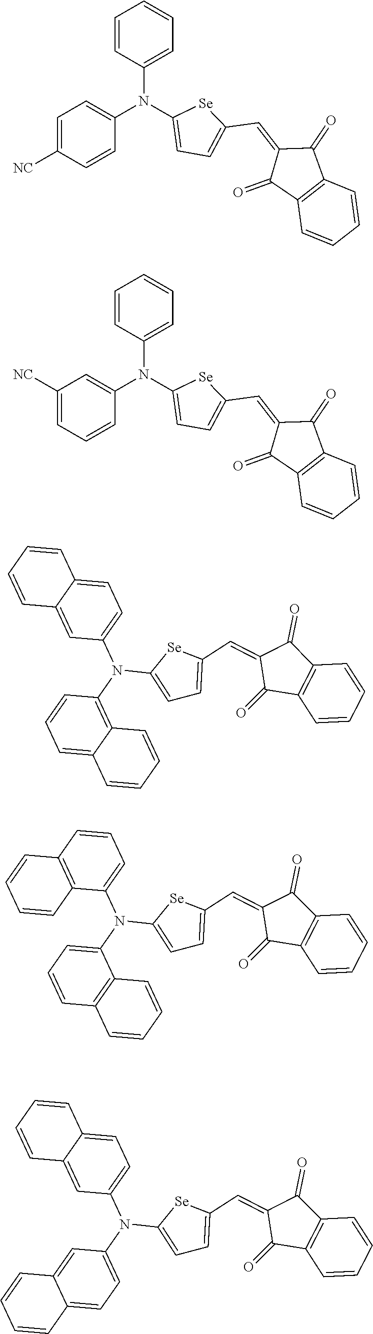

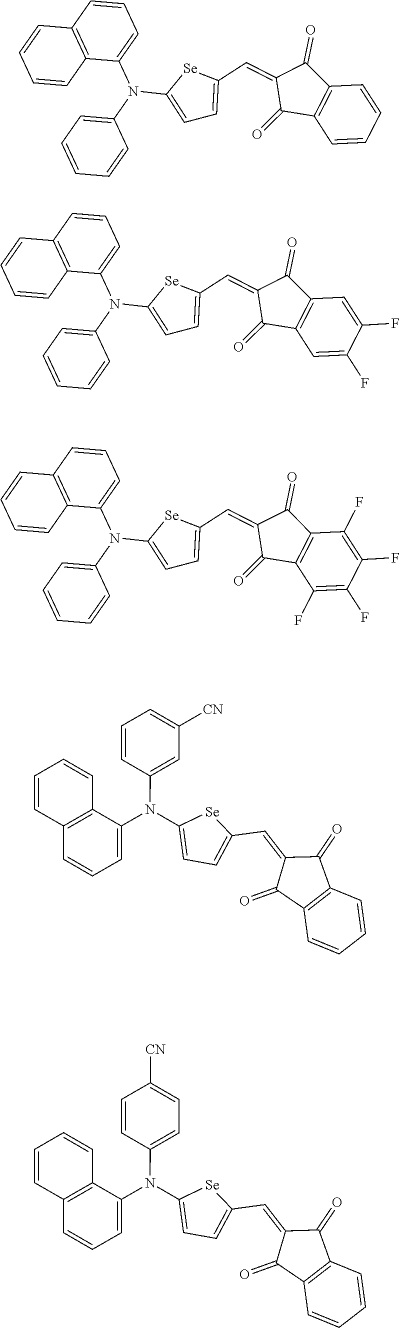

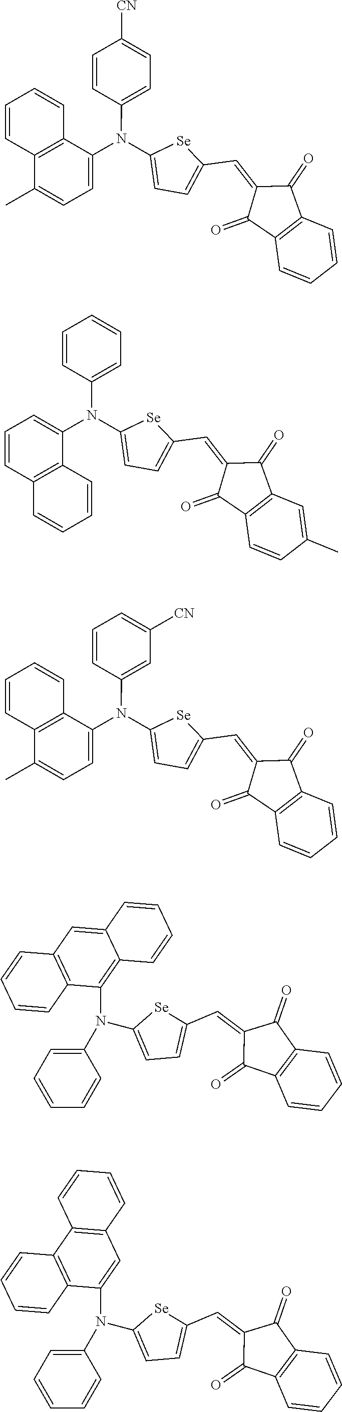

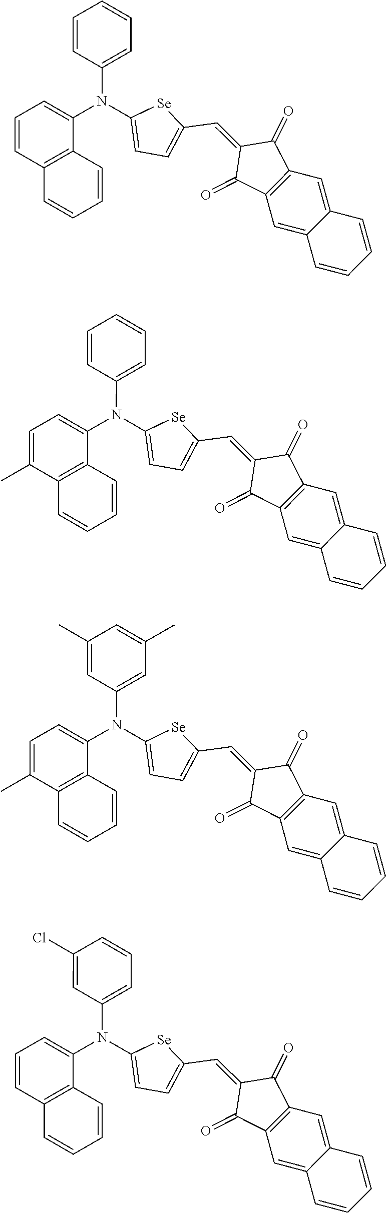

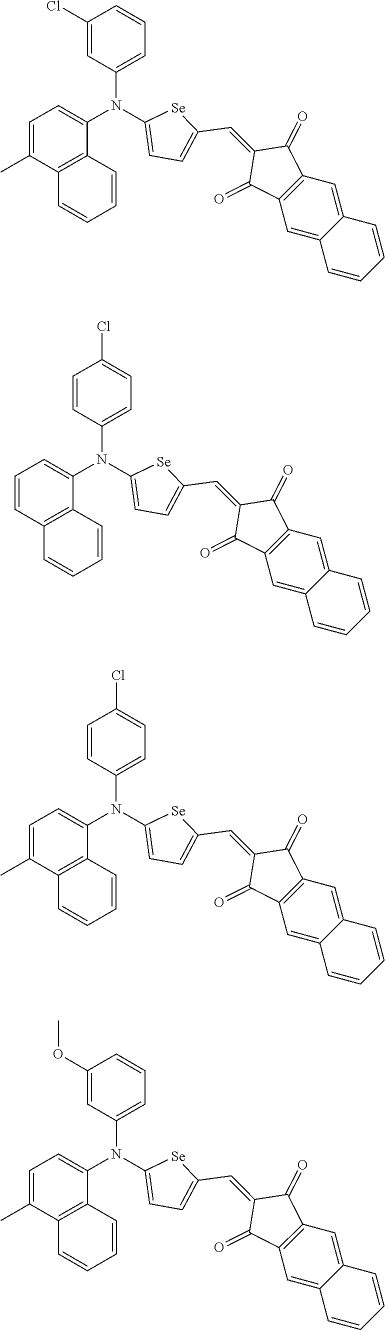

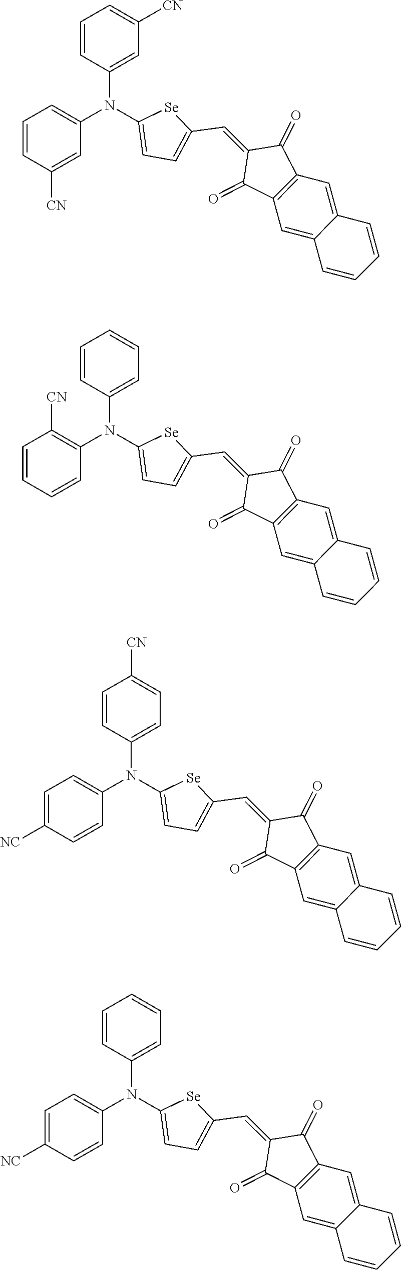

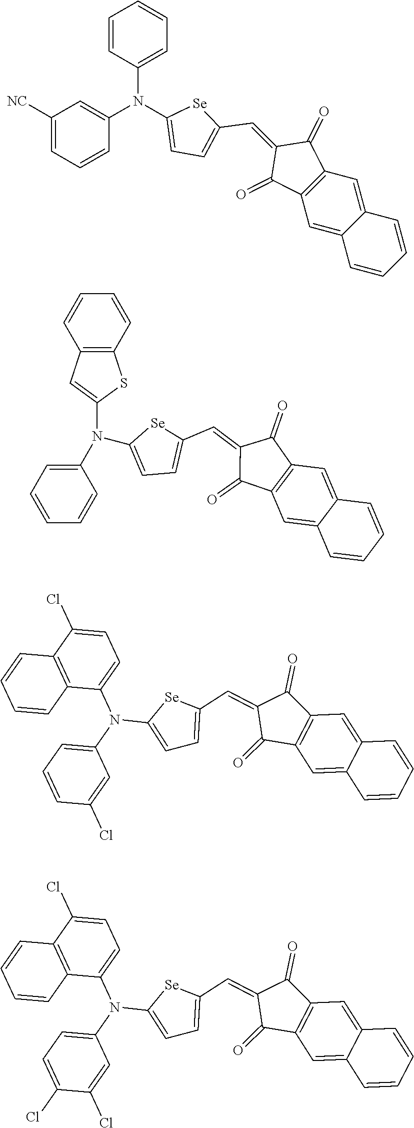

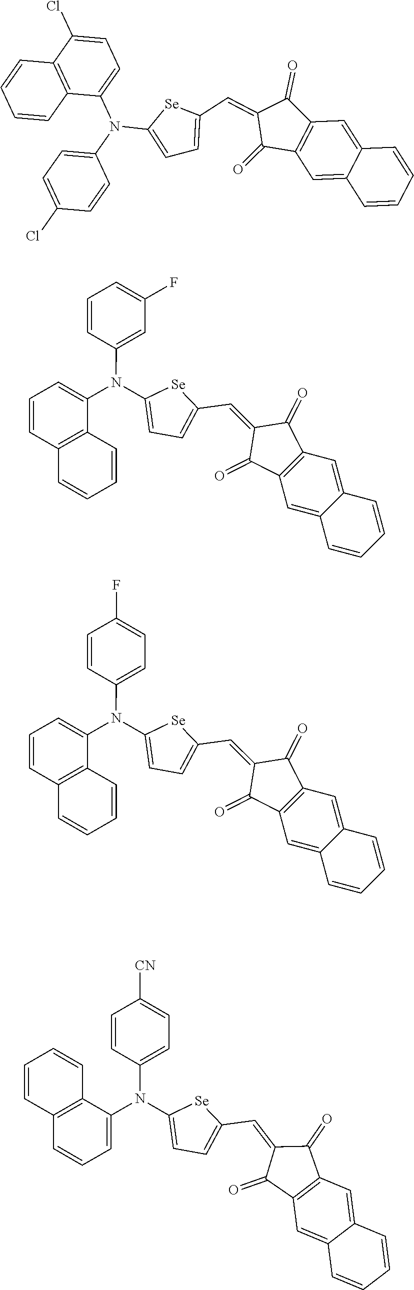

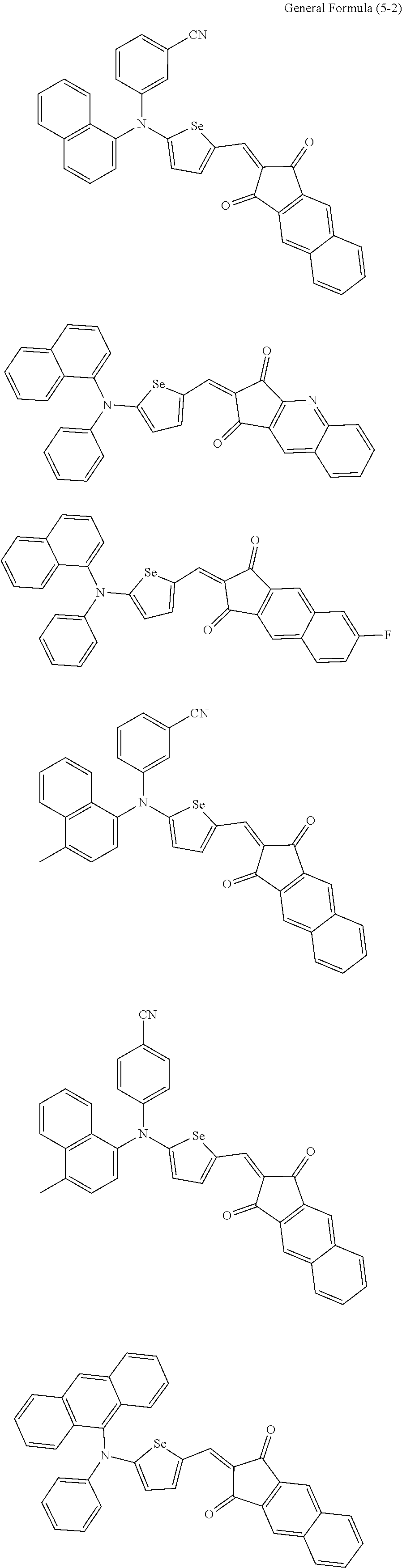

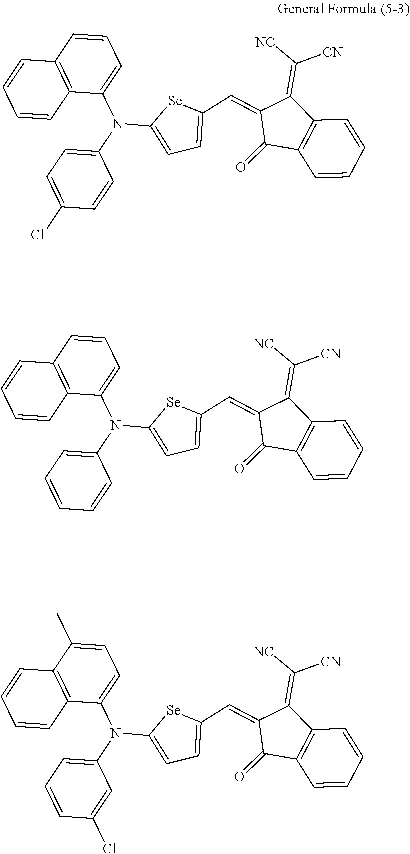

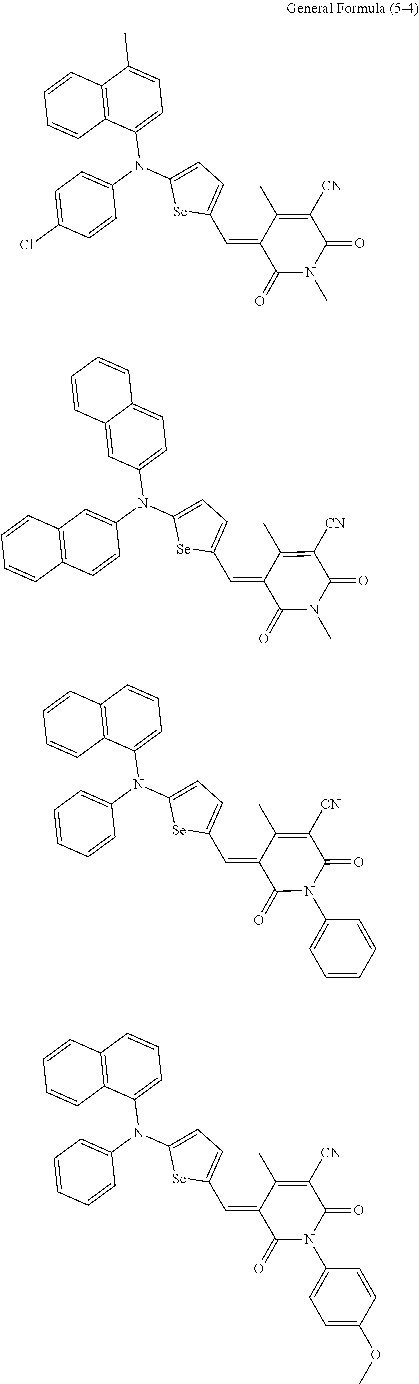

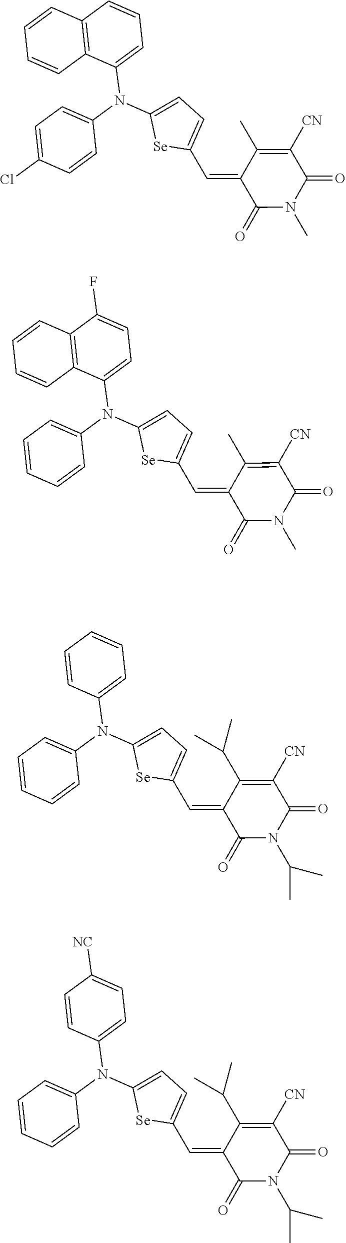

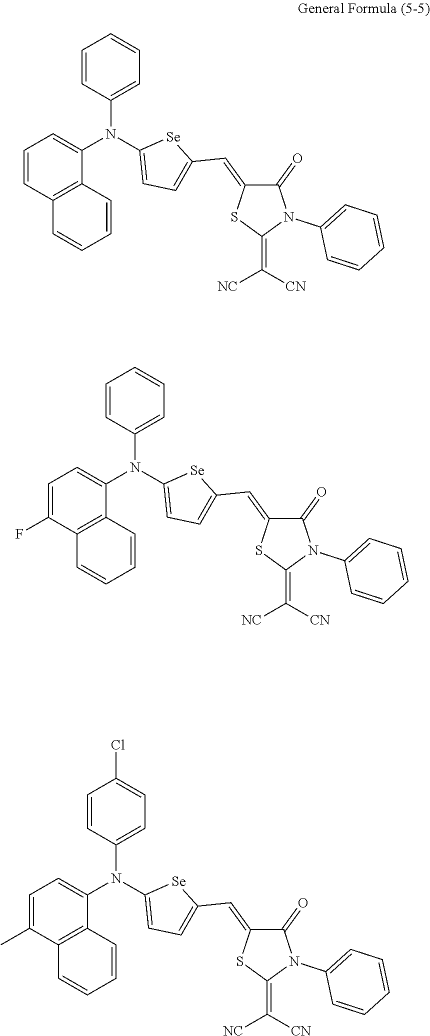

A molecular weight of the second compound is not particularly limited, but is preferably 300 to 1,200. In a case where the molecular weight is 1,200 or less, the vapor deposition temperature does is not high, and the decomposition of the compound hardly occurs. In a case where the molecular weight is 300 or more, the glass transition point of a deposited film is not low, and heat resistance of the photoelectric conversion element is improved.

A maximal absorption wavelength of the second compound is preferably 490 to 600 nm, more preferably 510 to 590 nm, and still more preferably 530 to 590 nm.

From the viewpoint of matching energy levels with the n-type semiconductor material described later, the second compound is preferably a compound having an ionization potential of 5.0 to 6.2 eV in a single film, more preferably a compound having an ionization potential of 5.2 to 6.1 eV, and still more preferably a compound having an ionization potential of 5.4 to 6.0 eV.

In the present specification, the ionization potential is a value measured by AC-2 of a photoelectron spectrometer manufactured by RIKEN KEIKI CO., LTD. for the single film of the compound.

A content of the second compound with respect to the entire photoelectric conversion film (=(Film thickness of second compound in terms of single layer/Film thickness of entire photoelectric conversion film)×100) is preferably 5% to 70% by volume, more preferably 10% to 50% by volume, and still more preferably 15% to 40% by volume.

A ratio of the content of the second compound to the content of the first compound in the entire photoelectric conversion film (Film thickness of second compound in terms of single layer/Film thickness of first compound in terms of single layer) is preferably 10/90 to 90/10, more preferably 30/70 to 70/30, and still more preferably 40/60 to 60/40.

<n-Type Semiconductor Material>

It is preferable that the photoelectric conversion film contains an n-type semiconductor material as a component other than the first compound and the second compound. The n-type semiconductor material is an accentor-type organic semiconductor material (compound), and refers to an organic compound having a property of easily accepting an electron.

More specifically, the n-type semiconductor material refers to an organic compound having excellent electron transport properties than the first compound and the second compound described above. In addition, it is preferable that the n-type semiconductor material has a large electron affinity with respect to both the first compound and the second compound described above.

In the present specification, the electron transport properties (electron carrier mobility) of the compound can be evaluated by, for example, a time-of-flight method (TOF method) or by using a field effect transistor element.

An electron carrier mobility of the n-type semiconductor material is preferably 10⁻⁴ cm²/V·s or more, more preferably 10⁻³ cm²/V·s or more, and still more preferably 10⁻² cm²/V·s or more. The upper limit of the above-described electron carrier mobility is not particularly limited, but for example, from the viewpoint of suppressing flow of a trace amount of current without light irradiation, it is preferably 10 cm²/V·s or less.

In the present specification, a value (value multiplied by −1) of a reciprocal number of LUMO value obtained by the calculation of B3LYP/6-31G(d) using Gaussian '09 (software manufactured by Gaussian, Inc.) as a value of the electron affinity.

The electron affinity of the n-type semiconductor material is preferably 3.0 to 5.0 eV.

Examples of the n-type semiconductor material include fullerenes selected from the group consisting of a fullerene and derivatives thereof; fused aromatic carbocyclic compounds (for example, a naphthalene derivative, an anthracene derivative, a phenanthrene derivative, a tetracene derivative, a pyrene derivative, a perylene derivative, a fluoranthene derivative, and the like); heterocyclic compounds with a 5- to 7-membered ring having at least one of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, and the like); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivatives and oxadiazole derivatives; anthraquinodimethane derivatives; diphenylquinone derivatives; bathocuproine, bathophenanthroline, and derivatives thereof; triazole compounds; distyrylarylene derivatives; metal complexes having a nitrogen-containing heterocyclic compound as a ligand; silole compounds; and compounds described in paragraphs [0056] and [0057] of JP2006-100767A.

Among these, the n-type semiconductor material preferably contains fullerenes selected from the group consisting of a fullerene and a derivative of fullerene.

Here, in a case where the photoelectric conversion element contains the above-described fullerenes, the element performance is expected to be more excellent; but the fullerenes have an absorption wavelength in a blue region, which deteriorates the selectivity to green light and may not have a favorable effect on the effect of the present invention.

However, it has been confirmed that the effect of the present invention can be obtained even in a case where the photoelectric conversion element further contains the fullerenes; and the present inventors speculate that, although the detailed mechanism is unknown, the first compound has an asymmetric structure, which also affects the aggregation of the fullerenes, and the absorption of blue light derived from the fullerenes can also be suppressed.

Examples of the fullerene include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the derivatives of fullerene include compounds in which a substituent is added to the above-described fullerenes. The substituent is preferably an alkyl group, an aryl group, or a heterocyclic group. As the derivatives of fullerene, compounds described in JP2007-123707A are preferable.

In a case where the n-type semiconductor material contains the fullerenes, a content of the fullerenes to the total content of the n-type semiconductor materials in the photoelectric conversion film (=(Film thickness of fullerenes in terms of single layer/Film thickness of total n-type semiconductor material in terms of single layer)×100) is preferably 15% to 100% by volume and more preferably 35% to 100% by volume.

An organic coloring agent may be used as the n-type semiconductor material, in place of the n-type semiconductor material described above or together with the n-type semiconductor material described above.

By using an organic coloring agent as the n-type semiconductor material, it is easy to control an absorption wavelength (maximal absorption wavelength) of the photoelectric conversion element to be within any wavelength range.

Examples of the above-described organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a fulgide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex dye, compounds described in paragraphs [0083] to [0089] of JP2014-82483A, compounds described in paragraphs [0029] to [0033] of JP2009-167348A, compounds described in paragraphs [0197] to [0227] of JP2012-77064A, compounds described in paragraphs [0035] to [0038] of WO2018/105269A, compounds described in paragraphs [0041] to [0043] of WO2018/186389A, compounds described in paragraphs [0059] to [0062] of WO2018/186397A, compounds described in paragraphs [0078] to [0083] of WO2019/009249A, compounds described in paragraphs [0054] to [0056] of WO2019/049946A, compounds described in paragraphs [0059] to [0063] of WO2019/054327A, and compounds described in paragraphs [0086] to [0087] of WO2019/098161A.

In a case where the n-type semiconductor material contains the organic coloring agent, a content of the organic coloring agent described above to the total content of the n-type semiconductor materials in the photoelectric conversion film (=(Film thickness of organic coloring agent in terms of single layer/Film thickness of total n-type semiconductor materials in terms of single layer)×100) is preferably 15% to 100% by volume and more preferably 35% to 100% by volume.

A molecular weight of the n-type semiconductor material is preferably 200 to 1,200 and more preferably 200 to 1,000.

It is preferable that the photoelectric conversion film has a bulk heterojunction structure formed in a state in which the first compound and/or the second compound, and the n-type semiconductor material are mixed with each other.

In addition, it is also preferable that the photoelectric conversion film includes a mixed layer which has a bulk heterojunction structure formed in a state in which the first compound, the second compound, and the n-type semiconductor material are mixed with each other.

The bulk heterojunction structure herein is a layer in which materials constituting the photoelectric conversion film (for example, the first compound and the n-type semiconductor material, the second compound and the n-type semiconductor material, or the first compound, the second compound, and the n-type semiconductor material) are mixed and dispersed in the photoelectric conversion film.

A content of the n-type semiconductor material with respect to the entire photoelectric conversion film (=(Film thickness of n-type semiconductor material in terms of single layer/Film thickness of entire photoelectric conversion film)×100) is preferably 5% to 70% by volume, more preferably 10% to 50% by volume, still more preferably 15% to 40% by volume, and most preferably 20% to 30% by volume.

From the viewpoint of responsiveness of the photoelectric conversion element, the content of the specific compound with respect to the total content of the specific compound (collectively referring to the first compound and the second compound) and the n-type semiconductor material in the entire photoelectric conversion film (=Total film thickness of specific compound in terms of single layer/(Total film thickness of specific compound in terms of single layer+Film thickness of n-type semiconductor material in terms of single layer)×100) is preferably 40% to 90% by volume and more preferably 60% to 90% by volume.

In addition, in a case where the photoelectric conversion film contains a p-type semiconductor material described later, the content of the specific compound in the entire photoelectric conversion film (=Total film thickness of specific compound in terms of single layer/(Total film thickness of specific compound in terms of single layer+Film thickness of n-type semiconductor material in terms of single layer+Film thickness of p-type semiconductor material in terms of single layer)×100) is preferably 15% to 75% by volume and more preferably 25% to 75% by volume.

It is preferable that the photoelectric conversion film is substantially composed of only the specific compound, the n-type semiconductor material contained as desired, and the p-type semiconductor material contained as desired. The term “substantially” is intended to mean that the total content of the specific compound, the n-type semiconductor material, and the p-type semiconductor material is 95% by mass or more with respect to the total mass of the photoelectric conversion film.

The n-type semiconductor material contained in the photoelectric conversion film may be used alone, or two or more thereof may be used in combination.

<p-Type Semiconductor Material>

It is preferable that the photoelectric conversion film contains a p-type semiconductor material as a component other than the first compound and the second compound.

The p-type semiconductor material is a donor-type organic semiconductor material (compound), and refers to an organic compound having a property of easily donating an electron.

More specifically, the p-type semiconductor material refers to an organic compound having excellent hole transport properties than the first compound and the second compound described above.

In the present specification, the hole transport properties (hole carrier mobility) of the compound can be evaluated by, for example, a time-of-flight method (TOF method) or by using a field effect transistor element.

The hole carrier mobility of the p-type semiconductor material is preferably 10⁻⁴ cm²/V·s or more, more preferably 10⁻³ cm²/V·s or more, and still more preferably 10⁻² cm²/V·s or more. The upper limit of the above-described hole carrier mobility is not particularly limited, but for example, from the viewpoint of suppressing flow of a trace amount of current without light irradiation, it is preferably 10 cm²/V·s or less.

In addition, it is also preferable that the p-type semiconductor material has a small ionization potential with respect to both the first compound and the second compound described above.

In addition, it is preferable that the p-type semiconductor material does not have absorption in the visible light region.

It is preferable that the photoelectric conversion film has a bulk heterojunction structure formed in a state in which the first compound and/or the second compound, the p-type semiconductor material, (and preferably the above-described n-type semiconductor material) are mixed with each other.

In addition, it is also preferable that the photoelectric conversion film has a bulk heterojunction structure formed in a state in which the first compound, the second compound, the p-type semiconductor material, (and preferably the above-described n-type semiconductor material) are mixed with each other.

The bulk heterojunction structure herein is a layer in which materials constituting the photoelectric conversion film (for example, the first compound and the p-type semiconductor material, the second compound and the p-type semiconductor material, the first compound, the second compound, and the p-type semiconductor material, the first compound, the n-type semiconductor material, and the p-type semiconductor material, the second compound, the n-type semiconductor material, and the p-type semiconductor material, or the first compound, the second compound, the n-type semiconductor material, and the p-type semiconductor material) are mixed and dispersed in the photoelectric conversion film.

Examples of the p-type semiconductor material include a triarylamine compound (for example, N,N′-bis(3-methylphenyl)-(1,1′-biphenyl)-4,4′-diamine (TPD), 4,4′-bis[N-(naphthyl)-N-phenyl-amino]biphenyl (α-NPD), compounds described in paragraphs [0128] to [0148] of JP2011-228614A, compounds described in paragraphs [0052] to [0063] of JP2011-176259A, compounds described in paragraphs [0119] to [0158] of JP2011-225544A, compounds described in paragraphs [0044] to [0051] of JP2015-153910A, compounds described in paragraphs [0086] to [0090] of JP2012-94660A, and the like), a pyrazoline compound, a styrylamine compound, a hydrazone compound, a polysilane compound, a thiophene compound (for example, a thienothiophene derivative, a dibenzothiophene derivative, a benzodithiophene derivative, a dithienothiophene derivative, a [1]benzothieno[3,2-b]thiophene (BTBT) derivative, a thieno[3,2-f:4,5-f′]bis[1]benzothiophene (TBBT) derivative, compounds described in paragraphs [0031] to [0036] of JP2018-14474A, compounds described in paragraphs [0043] to [0045] of WO2016/194630A, compounds described in paragraphs [0025] to [0037] and [0099] to [0109] of WO2017/159684A, compounds described in paragraphs [0029] to [0034] of JP2017-076766A, compounds described in paragraphs [0015] to [0025] of WO2018/207722A, compounds described in paragraphs [0045] to [0053] of JP2019-54228A, compounds described in paragraphs [0045] to [0055] of WO2019/058995A, compounds described in paragraphs [0063] to [0089] of WO2019/081416A, compounds described in paragraphs [0033] to [0036] of JP2019-80052A, compounds described in paragraphs [0044] to [0054] of WO2019/054125A, compounds described in paragraphs [0041] to [0046] of WO2019/093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having a nitrogen-containing heterocyclic compound as a ligand.

The p-type semiconductor material is also preferably a compound represented by Formula (p1), a compound represented by Formula (p2), a compound represented by Formula (p3), a compound represented by Formula (p4), or a compound represented by Formula (p5).

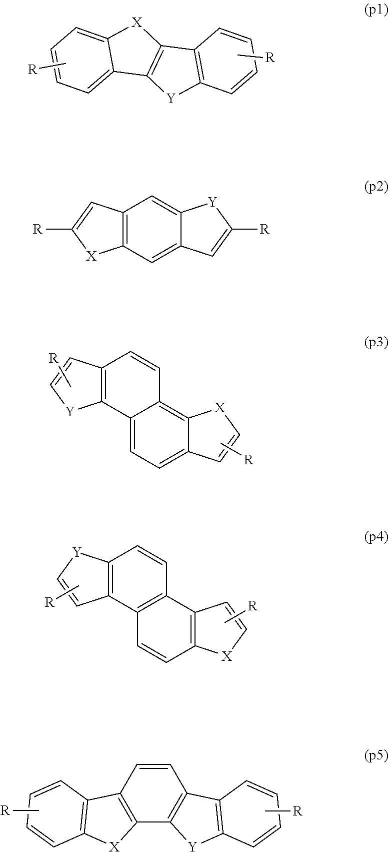

In Formulae (p1) to (p5), two R's each independently represent a hydrogen atom or a substituent (an alkyl group, an alkoxy group, a halogen atom, an alkylthio group, a (hetero)arylthio group, an alkylamino group, a (hetero)arylamino group, a (hetero)aryl group, or the like; these groups may further have a substituent if possible; for example, the (hetero)aryl group may be an aryl-aryl group (that is, a biaryl group, and at least one aryl group constituting the group may be a heteroaryl group) which may further have a substituent).

In addition, as R, a group represented by R in Formula (IX) of WO2019/081416A is also preferable.

X and Y each independently represent —CR²₂—, a sulfur atom, an oxygen atom, —NR²—, or —SiR²₂—.

R² represents a hydrogen atom, an alkyl group (preferably a methyl group or a trifluoromethyl group) which may have a substituent, an aryl group which may have a substituent, or a heteroaryl group which may have a substituent, and two or more R²'s may be the same or different from each other.

Compounds which can be used as the p-type semiconductor material are exemplified below.

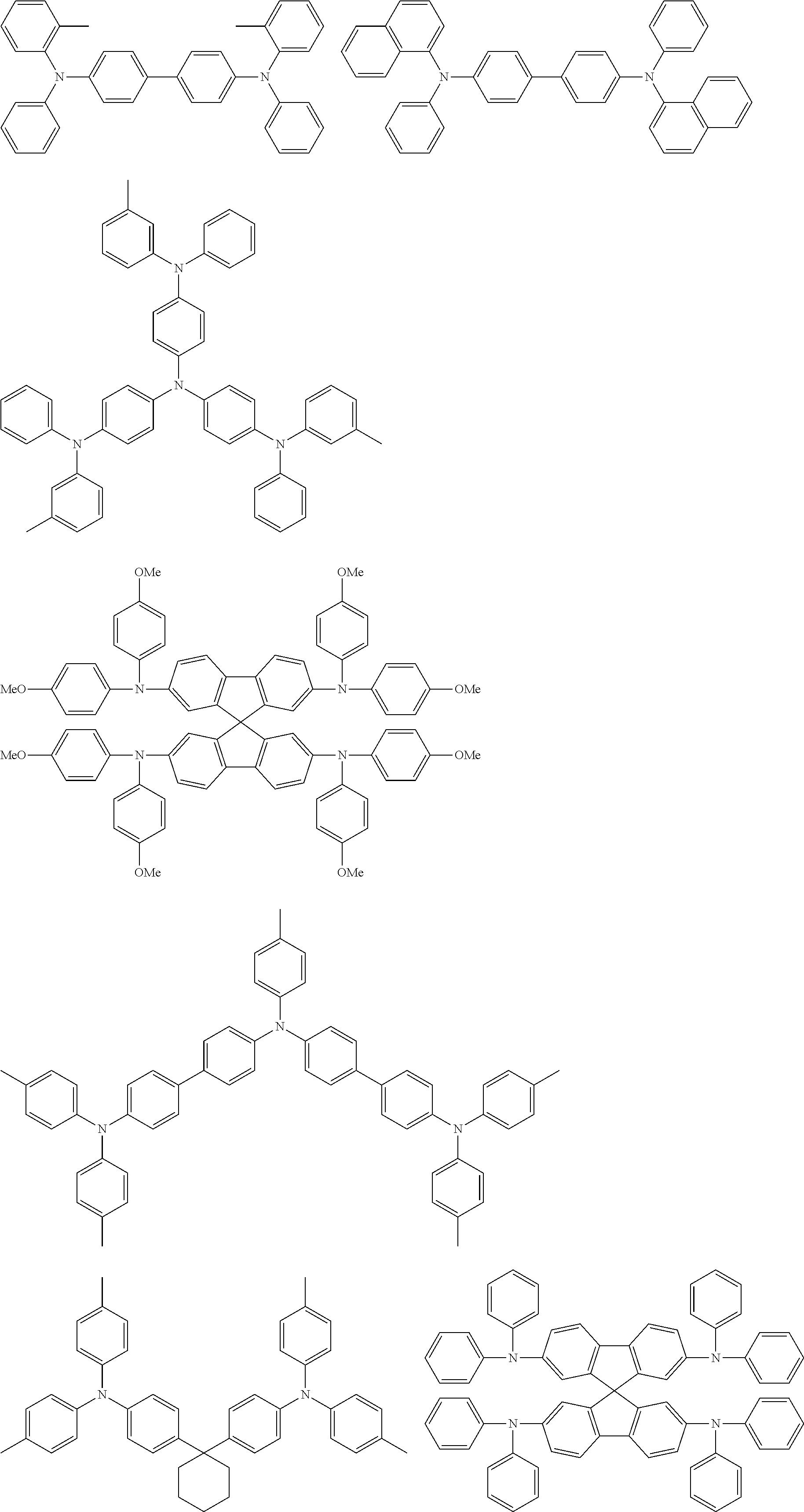

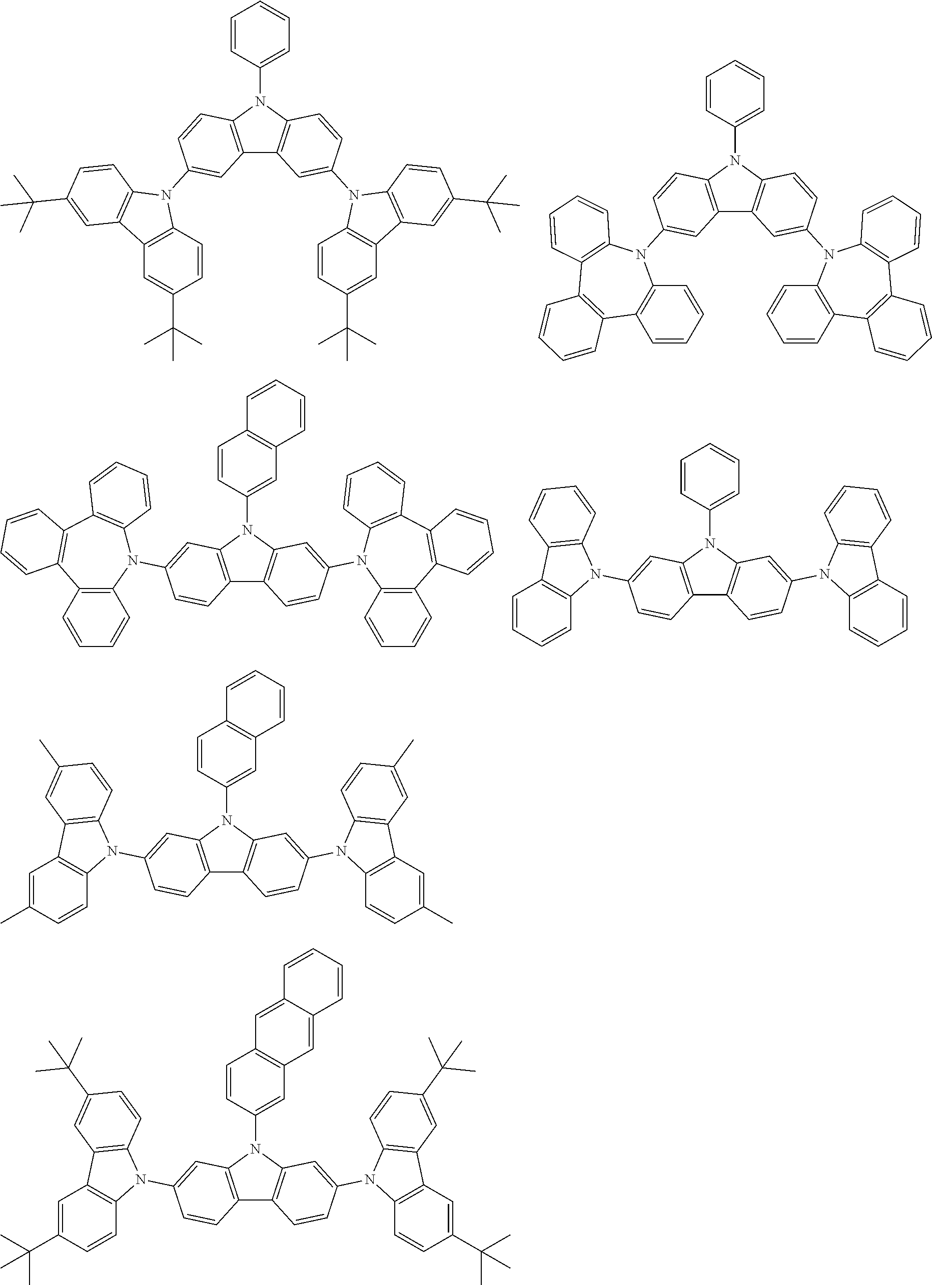

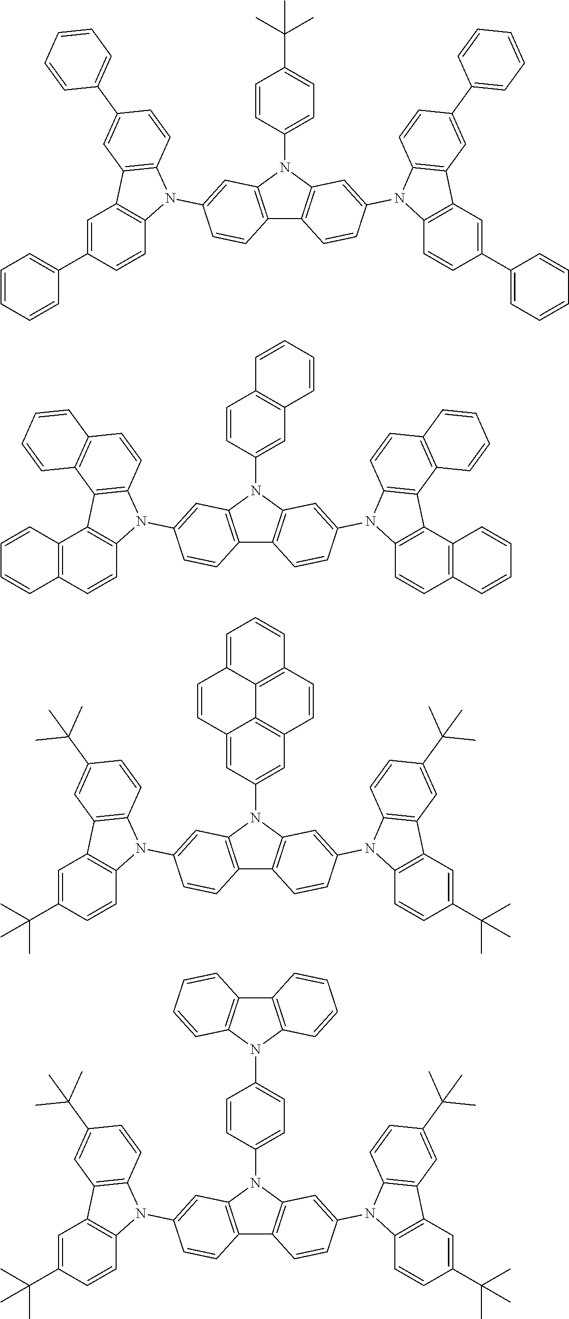

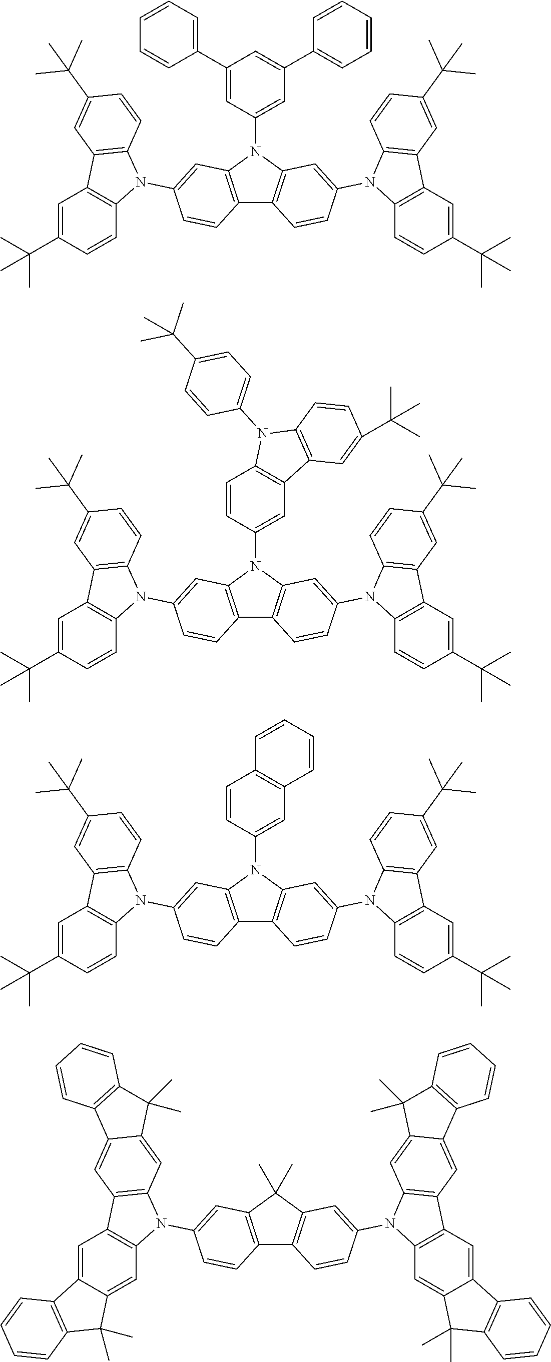

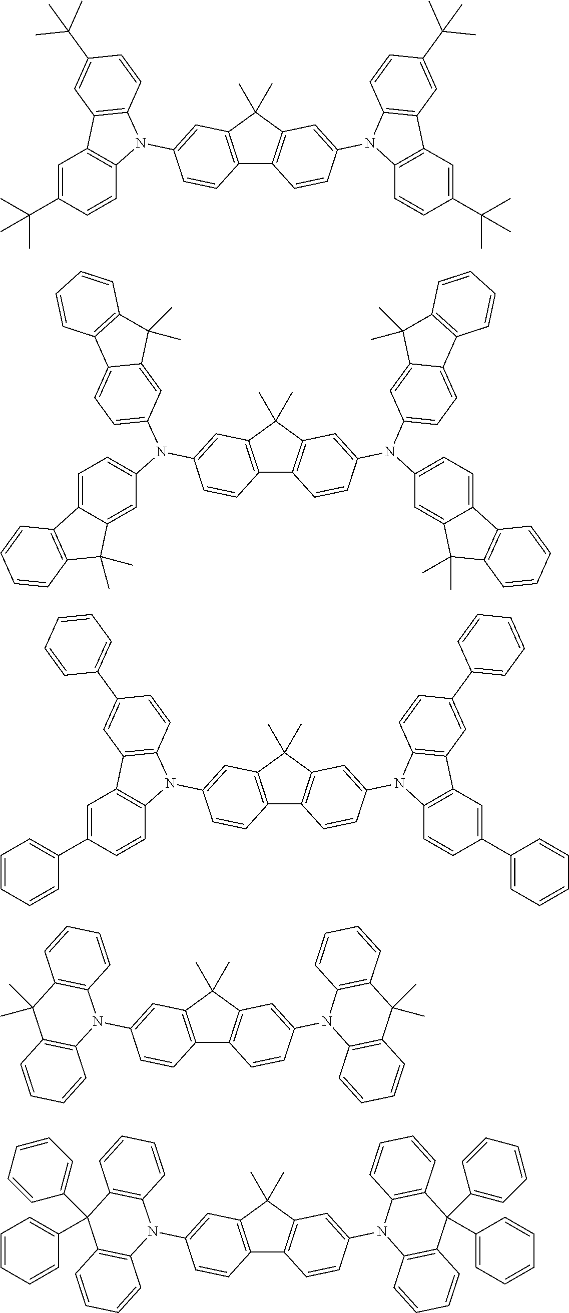

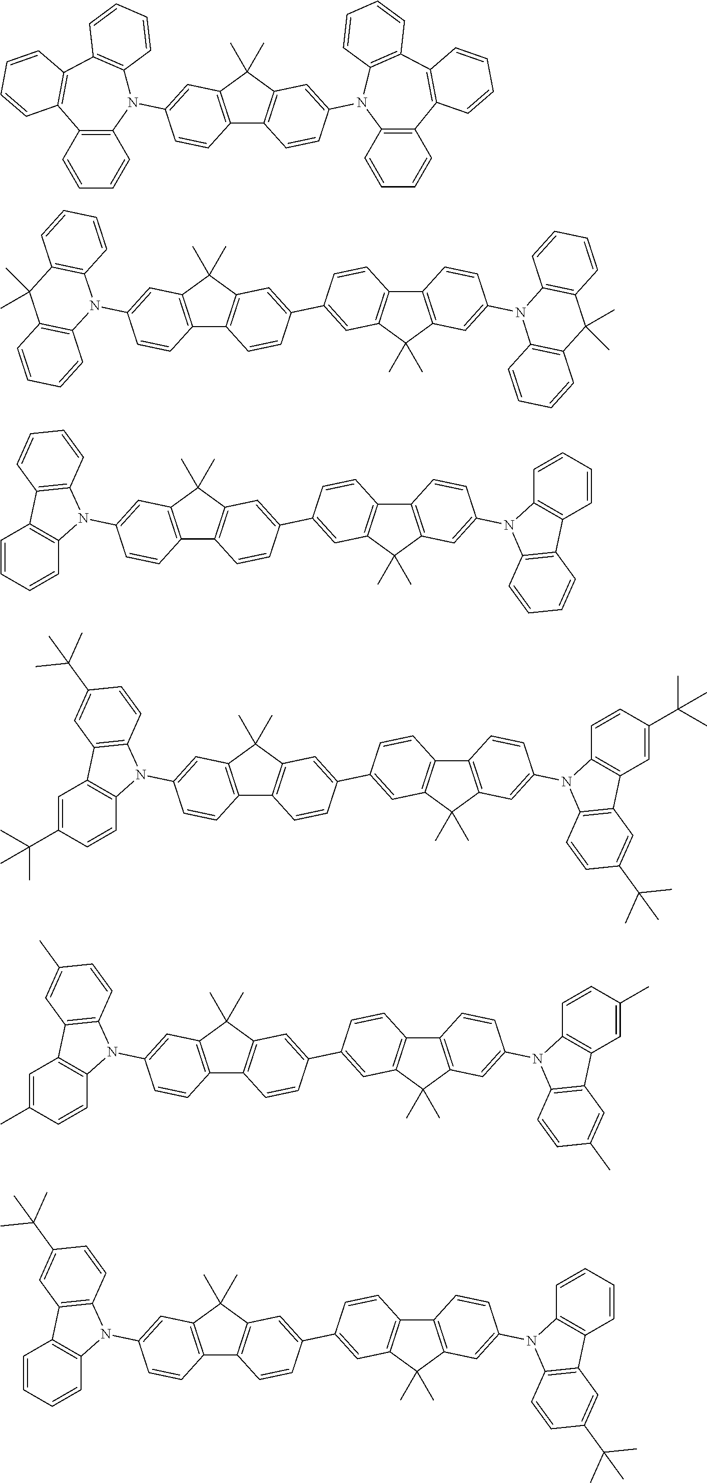

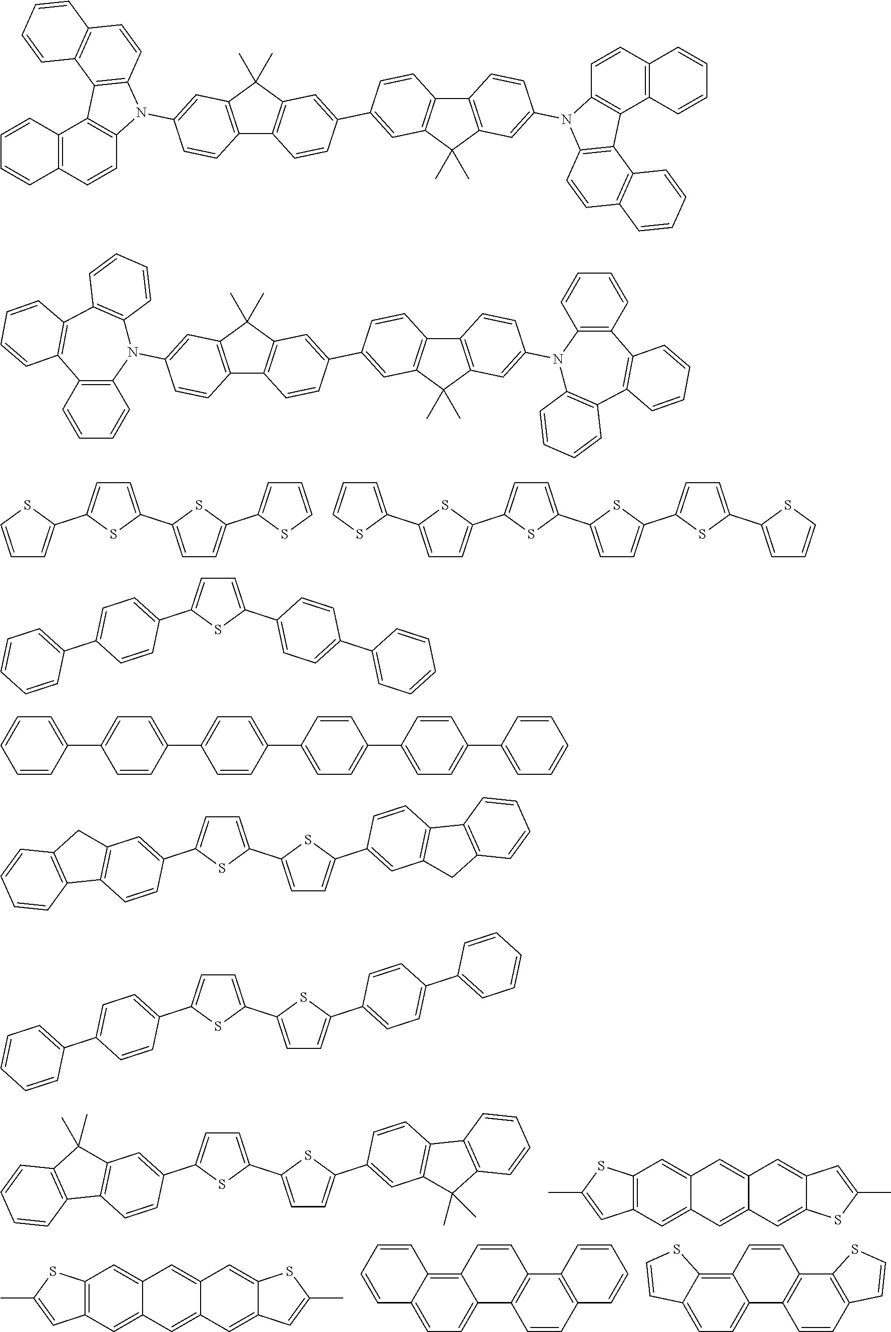

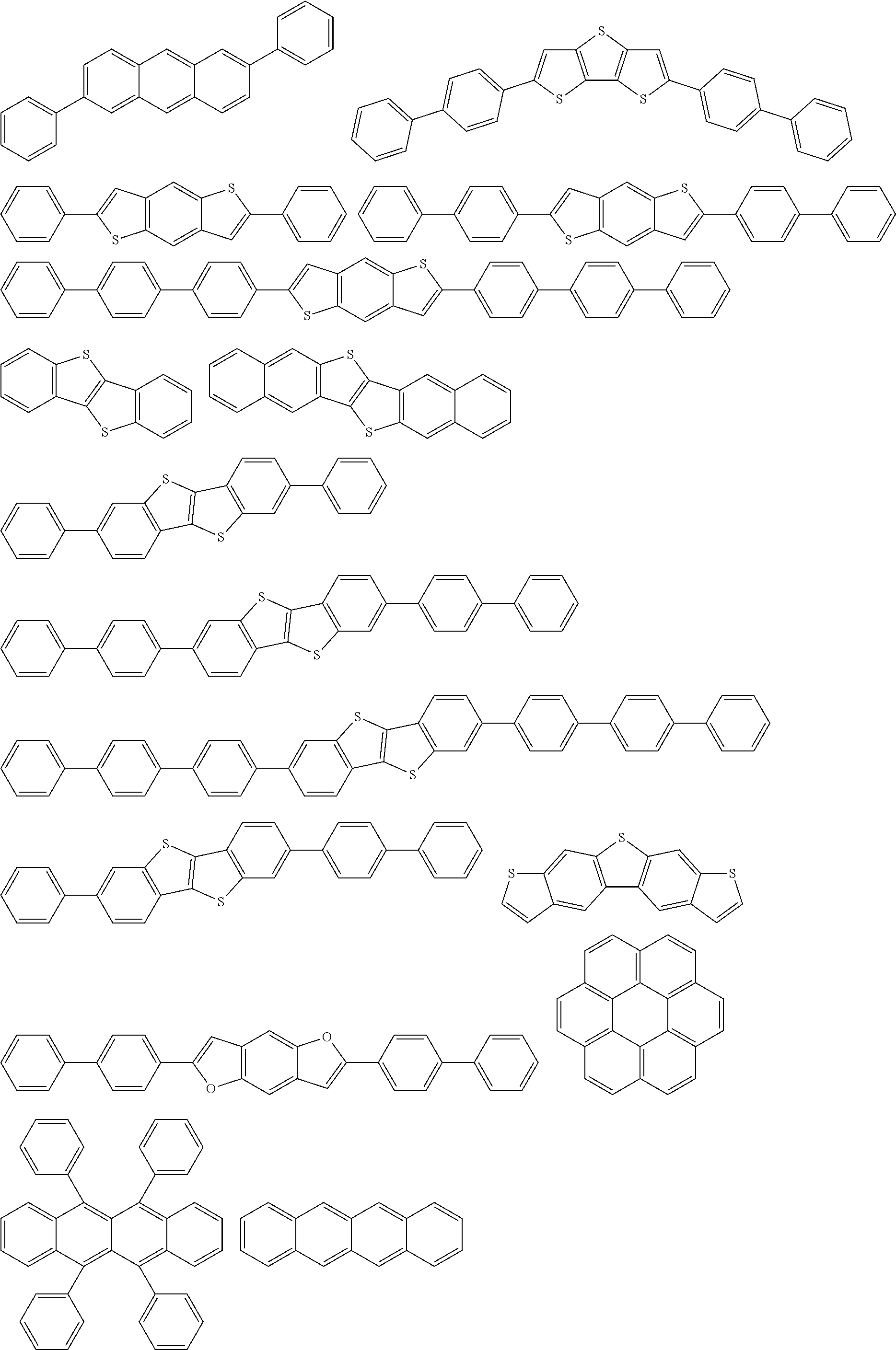

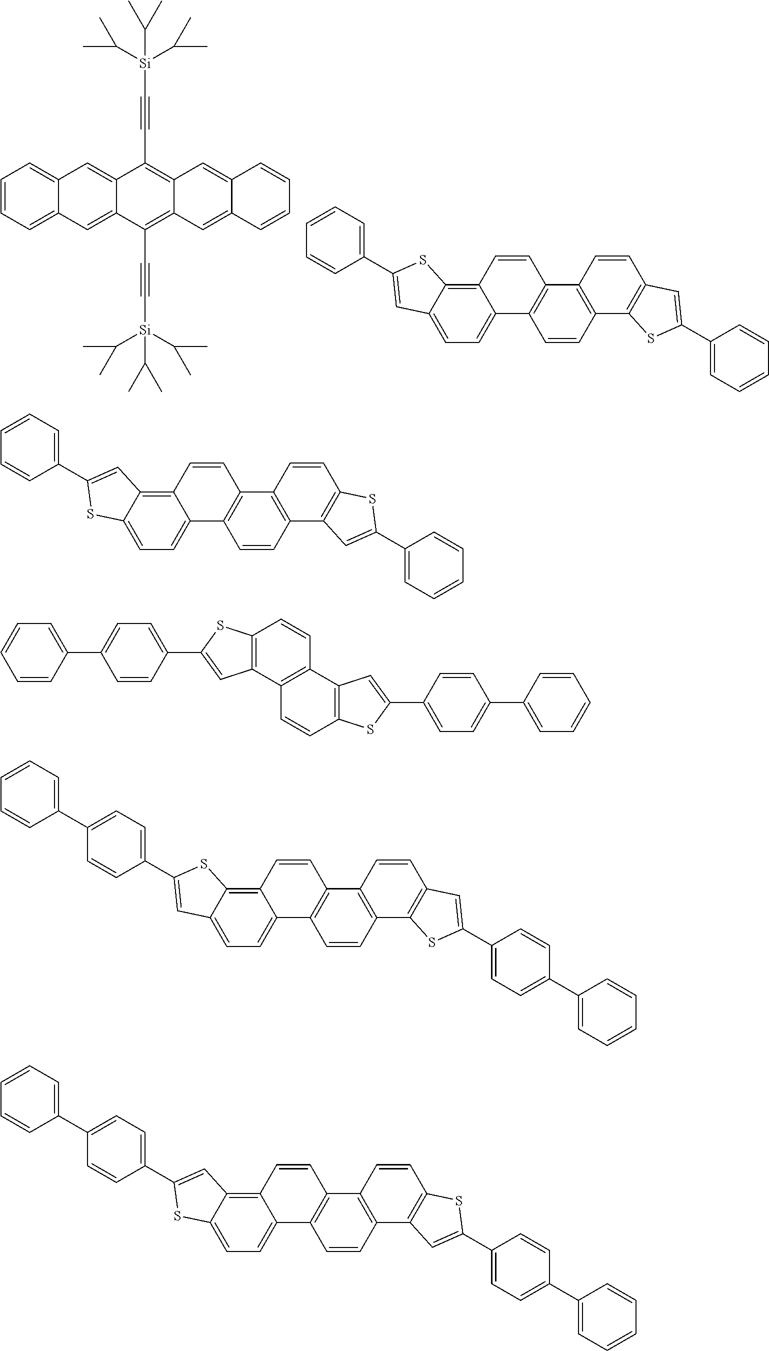

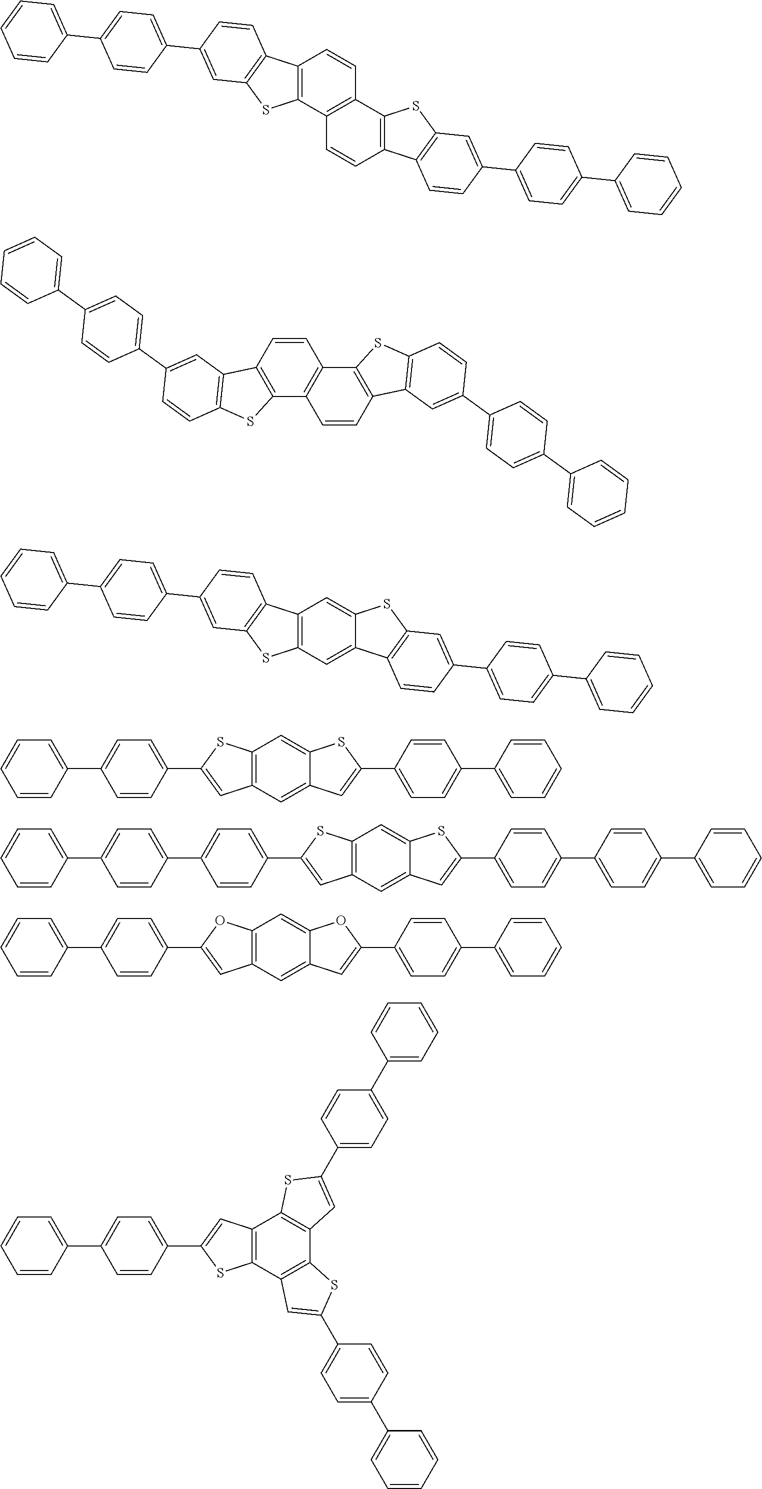

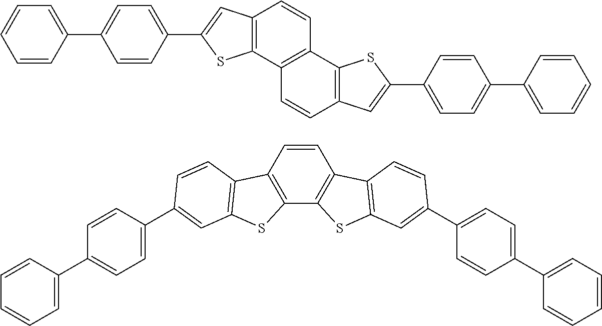

A content of the p-type semiconductor material with respect to the entire photoelectric conversion film (=(Film thickness of p-type semiconductor material in terms of single layer/Film thickness of entire photoelectric conversion film)×100) is preferably 5% to 70% by volume, more preferably 10% to 50% by volume, and still more preferably 15% to 40% by volume.

The p-type semiconductor material contained in the photoelectric conversion film may be used alone, or two or more thereof may be used in combination.

The photoelectric conversion film in the present invention is a non-luminescent film, and has a feature different from an organic light emitting diode (OLED). The non-luminescent film refers to a film having a light emission quantum efficiency of 1% or less, and the light emission quantum efficiency is preferably 0.5% or less and more preferably 0.1% or less.

### <Film Formation Method>

The photoelectric conversion film can be formed mostly by a dry film formation method. Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization. Among these, a vacuum vapor deposition method is preferable. In a case where the photoelectric conversion film is formed by the vacuum vapor deposition method, manufacturing conditions such as a degree of vacuum and a vapor deposition temperature can be set according to the conventional method.

A thickness of the photoelectric conversion film (in a case where the photoelectric conversion film is of a laminated type, a thickness of the entire photoelectric conversion film) is preferably 10 to 1,000 nm, more preferably 50 to 800 nm, and still more preferably 50 to 500 nm.

In addition, in a case where the photoelectric conversion film is of a laminated type, a thickness of each layer is independently preferably 5 to 500 nm, more preferably 25 to 400 nm, and still more preferably 25 to 250 nm.

### <Electrode>

The electrode (the upper electrode (transparent conductive film) 15 and the lower electrode (conductive film) 11) contains a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of a material constituting the upper electrode 15 include conductive metal oxides such as tin oxide doped with antimony, fluorine, or the like (antimony tin oxide (ATO) and fluorine doped tin oxide (FTO)), tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole. Among these, conductive metal oxides are preferable from the viewpoint of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is preferably 100 to 10000Ω/□, and the degree of freedom of the range of the film thickness that can be thinned is large. In addition, as the thickness of the upper electrode (the transparent conductive film) 15 is thinner, the amount of light that the upper electrode absorbs becomes smaller, and the light transmittance usually increases. The increase in the light transmittance causes an increase in light absorbance in the photoelectric conversion film and an increase in the photoelectric conversion ability, which is preferable. Considering suppression of leakage current, increase in resistance value of the thin film, and increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode does not have transparency and reflects light, depending on use. Examples of a material constituting the lower electrode 11 include conductive metal oxides such as tin oxide doped with antimony, fluorine, or the like (ATO and FTO), tin oxide, zinc oxide, indium oxide, indium tin oxide (ITO), and indium zinc oxide (IZO); metals such as gold, silver, chromium, nickel, titanium, tungsten, and aluminum; conductive compounds such as oxides or nitrides of these metals (for example, titanium nitride (TiN)); mixtures or laminates of these metals and conductive metal oxides; and organic conductive materials such as polyaniline, polythiophene, and polypyrrole.

The method of forming electrodes is not particularly limited, and can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance thermal vapor deposition method, a chemical reaction method (such as a sol-gel method), and a coating method with a dispersion of indium tin oxide.

### <Charge Blocking Film: Electron Blocking Film and Hole Blocking Film>

It is also preferable that the photoelectric conversion element according to the embodiment of the present invention includes one or more interlayers between the conductive film and the transparent conductive film, in addition to the photoelectric conversion film. Examples of the above-described interlayer include a charge blocking film. In a case where the photoelectric conversion element includes the film, characteristics (such as photoelectric conversion efficiency and responsiveness) of the obtained photoelectric conversion element are more excellent. Examples of the charge blocking film include an electron blocking film and a hole blocking film. The photoelectric conversion element preferably includes at least an electron blocking film as the interlayer.

Hereinafter, each film will be described in detail.

### (Electron Blocking Film)

The electron blocking film is a donor organic semiconductor material (compound).

The electron blocking film preferably has an ionization potential of 4.8 to 5.8 eV.

In addition, it is preferable that an ionization potential Ip(B) of the electron blocking film, an ionization potential Ip(1) of the first compound, and an ionization potential Ip(2) of the second compound satisfy relationships of Ip(B)≤Ip(1) and Ip(B)≤Ip(2).

As the electron blocking film, for example, a p-type organic semiconductor can be used. The p-type organic semiconductor may be used alone, or two or more thereof may be used in combination.

Examples of the p-type organic semiconductor include a triarylamine compound (for example, N,N′-bis(3-methylphenyl)-(1,1′-biphenyl)-4,4′-diamine (TPD), 4,4′-bis[N-(naphthyl)-N-phenyl-amino]biphenyl (α-NPD), compounds described in paragraphs [0128] to [0148] of JP2011-228614A, compounds described in paragraphs [0052] to [0063] of JP2011-176259A, compounds described in paragraphs [0119] to [0158] of JP2011-225544A, compounds described in paragraphs [0044] to [0051] of JP2015-153910A, compounds described in paragraphs [0086] to [0090] of JP2012-94660A, and the like), a pyrazoline compound, a styrylamine compound, a hydrazone compound, a polysilane compound, a thiophene compound (for example, a thienothiophene derivative, a dibenzothiophene derivative, a benzodithiophene derivative, a dithienothiophene derivative, a [1]benzothieno[3,2-b]thiophene (BTBT) derivative, a thieno[3,2-f:4,5-f′]bis[1]benzothiophene (TBBT) derivative, compounds described in paragraphs [0031] to [0036] of JP2018-14474A, compounds described in paragraphs [0043] to [0045] of WO2016/194630A, compounds described in paragraphs [0025] to [0037] and [0099] to [0109] of WO2017/159684A, compounds described in paragraphs [0029] to [0034] of JP2017-076766A, compounds described in paragraphs [0015] to [0025] of WO2018/207722A, compounds described in paragraphs [0045] to [0053] of JP2019-54228A, compounds described in paragraphs [0045] to [0055] of WO2019/058995A, compounds described in paragraphs [0063] to [0089] of WO2019/081416A, compounds described in paragraphs [0033] to [0036] of JP2019-80052A, compounds described in paragraphs [0044] to [0054] of WO2019/054125A, compounds described in paragraphs [0041] to [0046] of WO2019/093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having a nitrogen-containing heterocyclic compound as a ligand.

Examples of the p-type organic semiconductor include compounds having an ionization potential smaller than that of the n-type semiconductor material; and in a case where this condition is satisfied, the organic coloring agents exemplified as the n-type semiconductor material can be used.

In addition, a polymer material can also be used in the electron blocking film.

Specific examples of the polymer material include a polymer such as phenylenevinylene, fluorene, carbazole, indole, pyrene, pyrrole, picoline, thiophene, acetylene, and diacetylene, and derivatives thereof.

The electron blocking film may be configured by a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film, so that the photoelectric conversion efficiency increases. Examples of the inorganic material which can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### (Hole Blocking Film)

The hole blocking film is an acceptor-type organic semiconductor material (compound), and the above-described n-type semiconductor material can be used.

A method for manufacturing the charge blocking film is not particularly limited, and examples thereof include a dry film formation method and a wet film formation method. Examples of the dry film formation method include a vapor deposition method and a sputtering method. The vapor deposition method may be a physical vapor deposition (PVD) method or a chemical vapor deposition (CVD) method, and a physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method; and an inkjet method is preferable from the viewpoint of high-precision patterning.

Each thickness of the charge blocking films (the electron blocking film and the hole blocking film) is preferably 3 to 200 nm, more preferably 5 to 100 nm, and still more preferably 5 to 30 nm.

### <Substrate>

The photoelectric conversion element may further include a substrate. The type of the substrate to be used is not particularly limited, and examples thereof include a semiconductor substrate, a glass substrate, and a plastic substrate.

A position of the substrate is not particularly limited, and in general, the conductive film, the photoelectric conversion film, and the transparent conductive film are laminated on the substrate in this order.

### <Sealing Layer>

The photoelectric conversion element may further include a sealing layer. The performance of the photoelectric conversion material may deteriorate significantly due to the presence of deterioration factors such as water molecules. The deterioration can be prevented by coating and sealing the entirety of the photoelectric conversion film with a sealing layer such as diamond-like carbon (DLC) and ceramics such as metal oxide, metal nitride, or metal nitride oxide, which are dense and into which water molecules do not permeate.

The material of the sealing layer may be selected and the sealing layer may be produced according to the description in paragraphs [0210] to [0215] of JP2011-082508A.

### [Imaging Element]

Examples of the application of the photoelectric conversion element includes an imaging element including a photoelectric conversion element. The imaging element is an element which converts optical information of an image into the electric signal, and is usually an element in which a plurality of photoelectric conversion elements are arranged in a matrix on the same plane, optical signals are converted into electric signals in each photoelectric conversion element (a pixel), and the electric signals can be sequentially output to the outside of the imaging elements for each pixel. Therefore, each pixel is composed of one or more photoelectric conversion elements and one or more transistors.

The imaging element is mounted on an imaging element such as a digital camera and a digital video camera, an electronic endoscope, and an imaging module such as a cellular phone.

The photoelectric conversion element according to the embodiment of the present invention is also preferably used in an optical sensor including the photoelectric conversion element according to the embodiment of the present invention. The above-described photoelectric conversion element may be used alone as the optical sensor, or may be used as a line sensor in which the photoelectric conversion elements are linearly arranged or as a two-dimensional sensor in which the photoelectric conversion elements are arranged in plane.

### EXAMPLES

Hereinbelow, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### [Materials Used for Photoelectric Conversion Film]

Various components contained in the photoelectric conversion film are shown below.

### [First Compound or Second Compound]

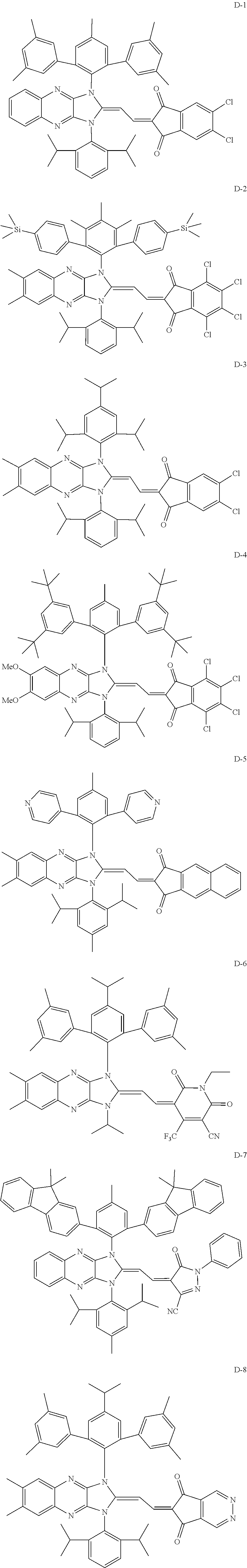

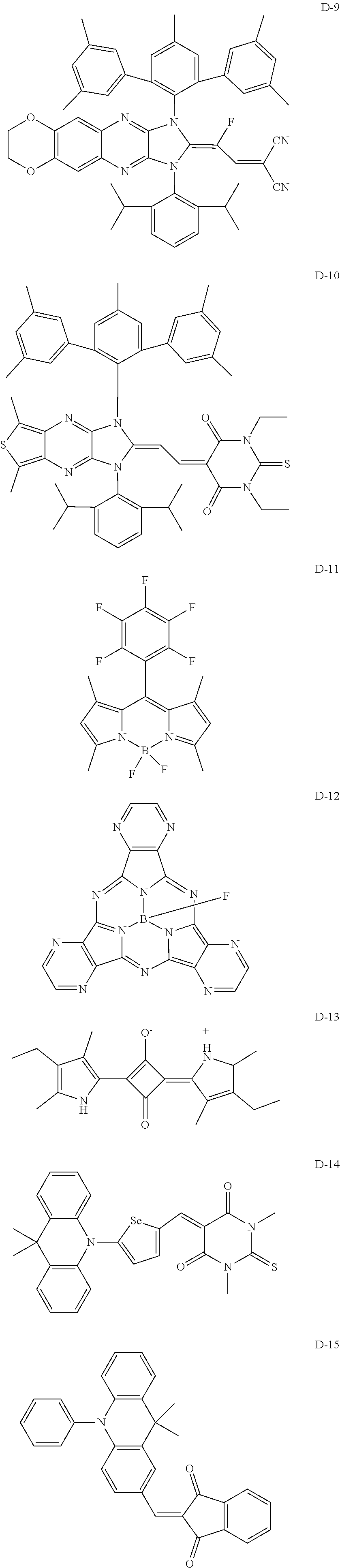

[n-Type Organic Semiconductor]
C60: fullerene (C₆₀)
   [p-Type Organic Semiconductor]

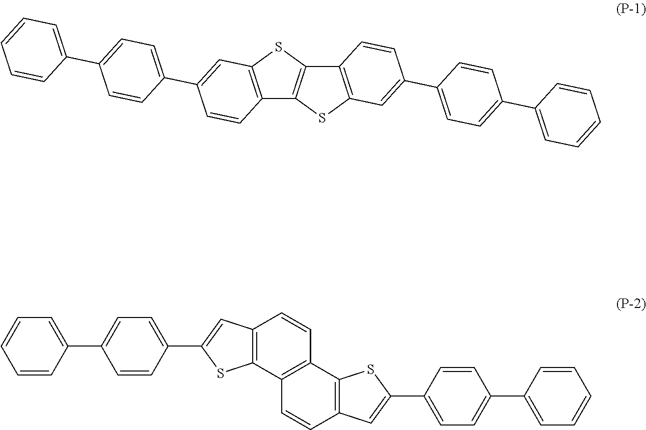 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

### [Evaluation]

A photoelectric conversion element was produced using each of the above-described materials, and each evaluation was carried out.

### <Production of Photoelectric Conversion Element>

A photoelectric conversion element having the form of FIG. 2 was produced using the various components shown above. Here, the photoelectric conversion element included a lower electrode 11, an electron blocking film 16A, a photoelectric conversion film 12, a hole blocking film 16B, and an upper electrode 15.

Specifically, an amorphous ITO was formed into a film on a glass substrate by a sputtering method to form the lower electrode 11 (thickness: 30 nm), and the compound (C-1) was further formed into a film on the lower electrode 11 by a vacuum thermal vapor deposition method to form the electron blocking film 16A (thickness: 30 nm).

Subsequently, in a state in which a temperature of the glass substrate was controlled to 25° C., the glass substrate was subjected to a vacuum vapor deposition method to form a film by co-vapor deposition of the first compound and the second compound shown in Table 1 on the electron blocking film 16A, and the n-type organic semiconductor (fullerene (C₆₀)) and the p-type organic semiconductor were subjected to co-vapor deposition by a vacuum vapor deposition method at a component ratio shown in Table 1 to form a film on the electron blocking film 16A. As a result, the photoelectric conversion film 12 having a bulk heterojunction structure with 240 nm was formed. In this case, a film formation rate of the photoelectric conversion film 12 was set to 1.0 Å/sec.

Furthermore, the compound (C-2) was vapor-deposited on the photoelectric conversion film 12 to form the hole blocking film 16B (thickness: 10 nm). Amorphous ITO was formed into a film on the hole blocking film 16B by a sputtering method to form the upper electrode 15 (transparent conductive film) (thickness: 10 nm). After the SiO film was formed as a sealing layer on the upper electrode 15 by a vacuum vapor deposition method, an aluminum oxide (Al₂O₃) layer was formed thereon by an atomic layer chemical vapor deposition (ALCVD) method. The obtained laminate was heated in a glove box at 150° C. for 30 minutes to obtain a photoelectric conversion element.

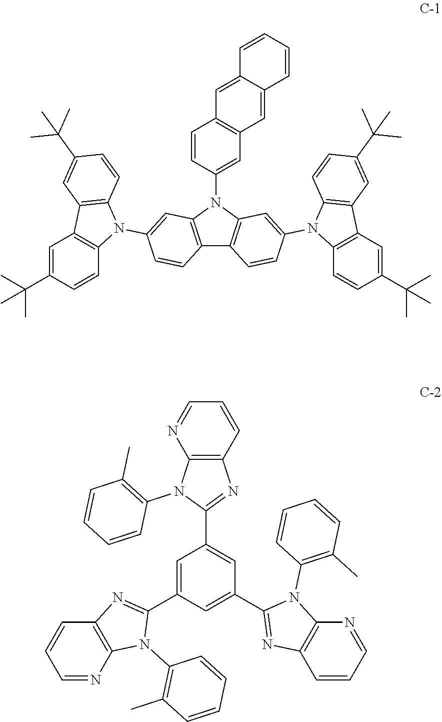 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### <Dark Current>

A dark current of each obtained photoelectric conversion element was measured by the following method. A voltage was applied to the lower electrode and the upper electrode of each of the photoelectric conversion elements with an electric field strength of 2.5×10⁵ V/cm, and a current value (dark current) in a dark place was measured. As a result, it was confirmed that all of the photoelectric conversion elements had a dark current of 50 nA/cm² or less, which indicates that all of the photoelectric conversion elements had a sufficiently low dark current.

### <Evaluation of Photoelectric Conversion Efficiency (External Quantum Efficiency)>

The driving of each of the obtained photoelectric conversion elements was confirmed. A voltage was applied to each photoelectric conversion element with an electric field strength of 2.0×10⁵ V/cm. Thereafter, light is emitted from the upper electrode (transparent conductive film) side to perform incident photon-to-current conversion efficiency (IPCE) measurement, and photoelectric conversion efficiency (external quantum efficiency) at each of a wavelength of 460 nm and a wavelength of 560 nm was extracted. The photoelectric conversion efficiency was measured using a constant energy quantum efficiency measuring device manufactured by OPTEL Co., LTD. The amount of light emitted was 50 W/cm².

In a case where the photoelectric conversion efficiency of the photoelectric conversion element of Example 1-1 at each wavelength was standardized as 1, the photoelectric conversion efficiency of each photoelectric conversion element was obtained and evaluated in the following categories based on the obtained photoelectric conversion efficiency. It was confirmed that each of the photoelectric conversion elements exhibited a photoelectric conversion efficiency of 40% or more at a measurement wavelength of 560 nm and had an external quantum efficiency of a certain level or higher as a photoelectric conversion element.

### (Evaluation Standard: Measurement Wavelength of 460 nm)

A: less than 1.1
B: 1.1 or more and less than 1.2
C: 1.2 or more

In practice, B or higher is preferable, and A is most preferable.

### (Evaluation Standard: Measurement Wavelength of 560 nm)

A: 0.9 or more
B: 0.7 or more and less than 0.9
C: less than 0.7

In practice, B or higher is preferable, and A is most preferable.

### <Evaluation of Response Speed (Responsiveness)>

Responsiveness of each of the obtained photoelectric conversion elements was evaluated. A voltage was applied to each photoelectric conversion element with an electric field strength of 2.0×10⁵ V/cm. Thereafter, a light emitting diode (LED) was turned on for an instant to emit light from the upper electrode (transparent conductive film) side, a photocurrent at a wavelength of 560 nm was measured with an oscilloscope, and a rise time until the signal intensity rose from 0% (immediately before a timing of moment when no irradiation was performed) to 97% was measured.

Next, the rise time of each photoelectric conversion element at a wavelength of 560 nm was obtained in a case where the rise time of the photoelectric conversion element of Example 1-1 was standardized as 1, and the responsiveness of each of the photoelectric conversion elements was evaluated in the following categories based on the obtained rise time.

### (Evaluation Standard)

AA: less than 0.9
A: 0.9 or more and less than 1.1
B: 1.1 or more and less than 2.0
C: 2.0 or more

In practice, B or higher is preferable, and AA is most preferable.

Table 1 shows each component used in the production of each photoelectric conversion element, the component ratio thereof, and the evaluation results of each photoelectric conversion element.

In Table 1, the numerical values in the column of “Component ratio” indicate the respective component ratios (volume ratios) in the order of first compound:second compound:n-type organic semiconductor:p-type organic semiconductor.

In Table 1, the column of “Difference in maximal absorption wavelength between compound 1 and compound 2” indicates the absolute value of the difference in maximal absorption wavelength between the first compound and the second compound, which is defined as follows.
A: 10 nm or less
B: more than 10 nm and 20 nm or less
C: more than 20 nm

TABLE 1

| | Material used for producing element | | | | | | | | |
|---|---|---|---|---|---|---|---|---|---|
| | | | | | Difference | | | | |
| | | | | | in maximal | | | | |
| | | | | | absorption | | Photoelectric | | |
| | | | | | wavelength | | conversion | | |
| | First | Second | n-type organic | p-type organic | between compound | Component | efficiency | | |
| | compound | compound | semiconductor | semiconductor | 1 and compound 2 | ratio | 460 nm | 560 nm | Responsiveness |
| Example 1-1 | (D-1) | (D-2) | C₆₀ | — | A | 1:1:1:0 | A | A | A |
| Example 1-2 | (D-1) | (D-2) | C₆₀ | (P-1) | A | 0.5:0.5:1:1 | A | A | AA |
| Example 1-3 | (D-1) | (D-2) | C₆₀ | (P-2) | A | 0.5:0.5:1:1 | A | A | AA |
| Example 1-4 | (D-1) | (D-3) | C₆₀ | (P-1) | A | 0.5:0.5:1:1 | A | A | AA |
| Example 1-5 | (D-1) | (D-4) | C₆₀ | (P-1) | A | 0.5:0.5:1:1 | A | A | AA |
| Example 1-6 | (D-2) | (D-4) | C₆₀ | (P-1) | A | 0.5:0.5:1:1 | A | A | AA |
| Example 1-7 | (D-1) | (D-5) | C₆₀ | (P-1) | A | 0.5:0.5:1:1 | A | A | AA |
| Example 1-8 | (D-1) | (D-6) | C₆₀ | (P-1) | A | 0.5:0.5:1:1 | A | A | AA |
| Example 1-9 | (D-1) | (D-7) | C₆₀ | (P-1) | A | 0.5:0.5:1:1 | A | A | AA |
| Example 1-10 | (D-1) | (D-8) | C₆₀ | (P-1) | A | 0.5:0.5:1:1 | A | A | AA |
| Example 1-11 | (D-1) | (D-9) | C₆₀ | (P-1) | A | 0.5:0.5:1:1 | A | A | AA |
| Example 1-12 | (D-1) | (D-10) | C₆₀ | (P-1) | B | 0.5:0.5:1:1 | A | A | A |
| Example 1-13 | (D-1) | (D-11) | C₆₀ | (P-1) | B | 0.9:0.1:1:1 | A | A | B |
| Example 1-14 | (D-1) | (D-12) | C₆₀ | (P-1) | A | 0.9:0.1:1:1 | A | A | B |
| Example 1-15 | (D-8) | (D-13) | C₆₀ | (P-1) | A | 0.9:0.1:1:1 | A | A | B |
| Example 1-16 | (D-1) | (D-14) | C₆₀ | (P-1) | A | 0.9:0.1:1:1 | A | A | B |
| Comparative | (D-8) | — | C₆₀ | — | — | 1:0:1:0 | C | C | C |
| Example 1-1 | | | | | | | | | |
| Comparative | (D-8) | — | C₆₀ | (P-1) | — | 1:0:1:1 | C | B | C |
| Example 1-2 | | | | | | | | | |
| Comparative | (D-1) | — | C₆₀ | (P-1) | — | 1:0:1:1 | B | A | C |
| Example 1-3 | | | | | | | | | |
| Comparative | (D-1) | (D-15) | C₆₀ | (P-1) | C | 0.9:0.1:1:1 | C | A | C |
| Example 1-4 | | | | | | | | | |

As shown in the table, it was found that the photoelectric conversion element according to the embodiment of the present invention had excellent responsiveness to green light.

From the comparison of Examples 1-2 to 1-12, it was found that, in a case where the absolute value of the difference between the maximal absorption wavelengths of the first compound and the second compound was 10 nm or less, the effect of the present invention was more excellent.

From the comparison of Examples 1-12 to 1-16, it was found that, in a case where the second compound was the compound represented by Formula (11), the effect of the present invention was more excellent.

From the comparison between Example 1-1 and Example 1-2, it was found that, in a case where the photoelectric conversion film in the photoelectric conversion element contained the p-type semiconductor material, the effect of the present invention was more excellent.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: hole blocking film

What is claimed is:

1. A photoelectric conversion element comprising, in the following order:
a conductive film;
a photoelectric conversion film; and
a transparent conductive film,
wherein the photoelectric conversion film contains a first compound represented by Formula (1) and a second compound which is a compound different from the first compound, and
a maximal absorption wavelength λ1 of the first compound and a maximal absorption wavelength λ2 of the second compound satisfy a relationship of an expression (X),
$$- 20 nm \leq λ 1 - λ 2 \leq 20 nm \qquad Expression ( X )$$ 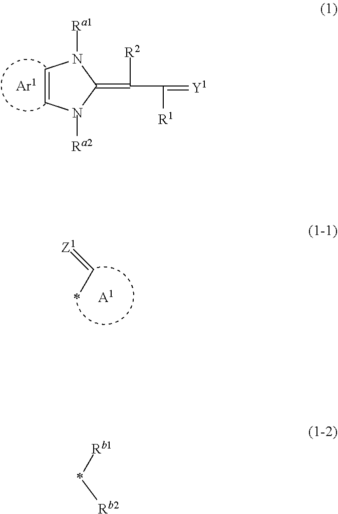
in Formula (1),
Ar¹ represents an aromatic ring which may have a substituent,
R^{a1} and R^{a2} each independently represent an aryl group which may have a substituent, —C(R^{L1})(R^{L2})(R^{L3}), or a heteroaryl group which may have a substituent, provided that R^{a1} and R^{a2} represent groups different from each other,
R^{L1} to R^{L3} each independently represent an alkyl group which may have a substituent, an aryl group which may have a substituent, a heteroaryl group which may have a substituent, or a hydrogen atom, and two or more of R^{L1} to R^{L3} each independently represent an alkyl group which may have a substituent, an aryl group which may have a substituent, or a heteroaryl group which may have a substituent, the alkyl group which may have a substituent, the aryl group which may have a substituent, and the heteroaryl group which may have a substituent, which are represented by R^{L1} to R^{L3}, may be bonded to each other to form a ring,
R¹ and R² each independently represent a hydrogen atom or a substituent, and
Y¹ represents a group represented by Formula (1-1) or a group represented by Formula (1-2),
in Formula (1-1),
Z¹ represents an oxygen atom, a sulfur atom, ═NR^{Z1}, or ═CR^{Z2}R^{Z3},
R^{Z1} represents a hydrogen atom or a substituent, R^{Z2} and R^{Z3} each independently represent a cyano group or —COOR^{Z4}, R^{Z4} represents an alkyl group which may have a substituent, an aryl group which may have a substituent, or a heteroaryl group which may have a substituent, and
A¹ represents a ring which contains at least two carbon atoms and may have a substituent,
in Formula (1-2),
R^{b1} and R^{b2} each independently represent a cyano group or —COOR^{b3},
R^{b3} represents an alkyl group which may have a substituent, an aryl group which may have a substituent, or a heteroaryl group which may have a substituent, and
* represents a bonding position.

2. The photoelectric conversion element according to claim 1,
wherein the first compound is a compound represented by Formula (2), 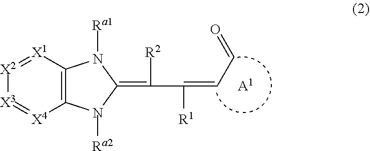
in Formula (2),
X¹ to X⁴ each independently represent a nitrogen atom or —CR^{c1}—,
R^{c1} represents a hydrogen atom or a substituent,
in a case of a plurality of R^{c1}'s, the plurality of R^{c1}'s may be bonded to each other to form a ring,
R^{a1} and R^{a2} each independently represent an aryl group which may have a substituent, —C(R^{L1})(R^{L2})(R^{L3}), or a heteroaryl group which may have a substituent, provided that R^{a1} and R^{a2} represent groups different from each other,
R^{L1} to R^{L3} each independently represent an alkyl group which may have a substituent, an aryl group which may have a substituent, a heteroaryl group which may have a substituent, or a hydrogen atom, and two or more of R^{L1} to R^{L3} each independently represent an alkyl group which may have a substituent, an aryl group which may have a substituent, or a heteroaryl group which may have a substituent, the alkyl group which may have a substituent, the aryl group which may have a substituent, and the heteroaryl group which may have a substituent, which are represented by R^{L1} to R^{L3}, may be bonded to each other to form a ring,
R¹ and R² each independently represent a hydrogen atom or a substituent, and
A¹ represents a ring which contains at least two carbon atoms and may have a substituent.

3. The photoelectric conversion element according to claim 2,
wherein X¹ and X⁴ in Formula (2) represent a nitrogen atom.

4. The photoelectric conversion element according to claim 1,
wherein the first compound is a compound represented by Formula (3), 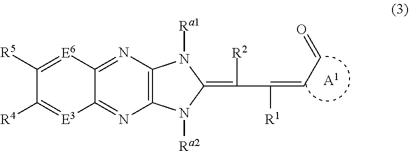
in Formula (3),
E³ represents a nitrogen atom or —CR³═,
E⁶ represents a nitrogen atom or —CR⁶═,
R¹ to R⁶ each independently represent a hydrogen atom or a substituent,
R³ and R⁴, R⁴ and R⁵, or R⁵ and R⁶ may be each independently bonded to each other to form a ring,
R^{a1} and R^{a2} each independently represent an aryl group which may have a substituent, —C(R^{L1})(R^{L2})(R^{L3}), or a heteroaryl group which may have a substituent, provided that R^{a1} and R^{a2} represent groups different from each other,
R^{L1} to R^{L3} each independently represent an alkyl group which may have a substituent, an aryl group which may have a substituent, a heteroaryl group which may have a substituent, or a hydrogen atom, and two or more of R^{L1} to R^{L3} each independently represent an alkyl group which may have a substituent, an aryl group which may have a substituent, or a heteroaryl group which may have a substituent, the alkyl group which may have a substituent, the aryl group which may have a substituent, and the heteroaryl group which may have a substituent, which are represented by R^{L1} to R^{L3}, may be bonded to each other to form a ring, and
A¹ represents a ring which contains at least two carbon atoms and may have a substituent.

5. The photoelectric conversion element according to claim 1,
wherein the second compound is a compound selected from the group consisting of a compound having an imidazoline skeleton, a pyrromethene boron complex, a subphthalocyanine compound, a squarylium compound, and a compound having a triarylamine skeleton.

6. The photoelectric conversion element according to claim 1,
wherein the second compound is a compound represented by Formula (11), 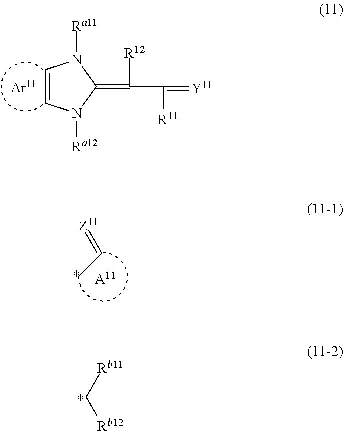
in Formula (11),
Ar¹¹ represents an aromatic ring which may have a substituent,
R^{a11} and R^{a12} each independently represent an aryl group which may have a substituent, —C(R^{L11})(R^{L12})(R^{L13}), or a heteroaryl group which may have a substituent, provided that R^{a11} and R^{a12} represent groups different from each other,
R^{L11} to R^{L13} each independently represent an alkyl group which may have a substituent, an aryl group which may have a substituent, a heteroaryl group which may have a substituent, or a hydrogen atom, and two or more of R^{L11} to R^{L13} each independently represent an alkyl group which may have a substituent, an aryl group which may have a substituent, or a heteroaryl group which may have a substituent, the alkyl group which may have a substituent, the aryl group which may have a substituent, and the heteroaryl group which may have a substituent, which are represented by R^{L11} to R^{L13}, may be bonded to each other to form a ring, and
R¹¹ and R¹² each independently represent a hydrogen atom or a substituent, and
Y¹¹ represents a group represented by Formula (11-1) or a group represented by Formula (11-2),
in Formula (11-1),
Z¹¹ represents an oxygen atom, a sulfur atom, ═NR^{Z11}, or ═CR^{Z12}R^{Z13}, R^{Z11} represents a hydrogen atom or a substituent, R^{Z12} and R^{Z13} each independently represent a cyano group or —COOR^{Z14}, R^{Z14} represents an alkyl group which may have a substituent, an aryl group which may have a substituent, or a heteroaryl group which may have a substituent, and
A¹¹ represents a ring which contains at least two carbon atoms and may have a substituent,
in Formula (11-2),
R^{b11} and R^{b12} each independently represent a cyano group or —COOR^{b13},
R^{b13} represents an alkyl group which may have a substituent, an aryl group which may have a substituent, or a heteroaryl group which may have a substituent, and
* represents a bonding position.

7. The photoelectric conversion element according to claim 1,
wherein the photoelectric conversion film further contains an n-type semiconductor material, and
the photoelectric conversion film has a bulk heterojunction structure formed in a state in which the first compound, the second compound, and the n-type semiconductor material are mixed with each other.

8. The photoelectric conversion element according to claim 7,
wherein the n-type semiconductor material includes fullerenes selected from the group consisting of a fullerene and derivatives of the fullerene.

9. The photoelectric conversion element according to claim 1,
wherein the photoelectric conversion film further contains an n-type semiconductor material and a p-type semiconductor material, and
the photoelectric conversion film has a bulk heterojunction structure formed in a state in which the first compound, the second compound, the n-type semiconductor material, and the p-type semiconductor material are mixed with each other.

10. The photoelectric conversion element according to claim 9,
wherein the n-type semiconductor material includes fullerenes selected from the group consisting of a fullerene and derivatives of the fullerene.

11. The photoelectric conversion element according to claim 9,
wherein the p-type semiconductor material has no absorption in a visible light region.

12. The photoelectric conversion element according to claim 1, further comprising:
one or more interlayers between the conductive film and the transparent conductive film, in addition to the photoelectric conversion film.

13. An imaging element comprising:
the photoelectric conversion element according to claim 1.

14. An optical sensor comprising:
the photoelectric conversion element according to claim 1.

15. The photoelectric conversion element according to claim 5,
wherein the second compound is a compound represented by Formula (11), 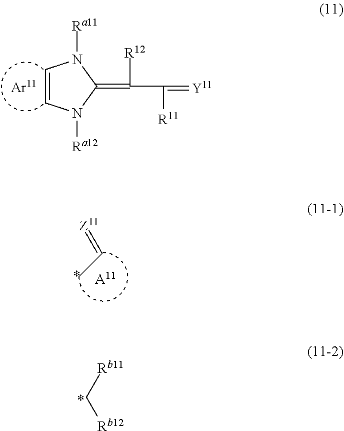
in Formula (11),
Ar¹¹ represents an aromatic ring which may have a substituent,
R^{a11} and R^{a12} each independently represent an aryl group which may have a substituent, —C(R^{L11})(R^{L12})(R^{L13}), or a heteroaryl group which may have a substituent, provided that R^{a11} and R^{a12} represent groups different from each other,
R^{L11} to R^{L13} each independently represent an alkyl group which may have a substituent, an aryl group which may have a substituent, a heteroaryl group which may have a substituent, or a hydrogen atom, and two or more of R^{L11} to R^{L13} each independently represent an alkyl group which may have a substituent, an aryl group which may have a substituent, or a heteroaryl group which may have a substituent, the alkyl group which may have a substituent, the aryl group which may have a substituent, and the heteroaryl group which may have a substituent, which are represented by R^{L11} to R^{L13}, may be bonded to each other to form a ring, and
R¹¹ and R¹² each independently represent a hydrogen atom or a substituent, and
Y¹¹ represents a group represented by Formula (11-1) or a group represented by Formula (11-2),
in Formula (11-1),
Z¹¹ represents an oxygen atom, a sulfur atom, ═NR^{Z11}, or ═CR^{Z12}R^{Z13}, R^{Z11} represents a hydrogen atom or a substituent, R^{Z12} and R^{Z13} each independently represent a cyano group or —COOR^{Z14}, R^{Z14} represents an alkyl group which may have a substituent, an aryl group which may have a substituent, or a heteroaryl group which may have a substituent, and
A¹¹ represents a ring which contains at least two carbon atoms and may have a substituent,
in Formula (11-2),
R^{b11} and R^{b12} each independently represent a cyano group or —COOR^{b13},
R^{b13} represents an alkyl group which may have a substituent, an aryl group which may have a substituent, or a heteroaryl group which may have a substituent, and
* represents a bonding position.

16. The photoelectric conversion element according to claim 5,
wherein the photoelectric conversion film further contains an n-type semiconductor material, and
the photoelectric conversion film has a bulk heterojunction structure formed in a state in which the first compound, the second compound, and the n-type semiconductor material are mixed with each other.

17. The photoelectric conversion element according to claim 16,
wherein the n-type semiconductor material includes fullerenes selected from the group consisting of a fullerene and derivatives of the fullerene.

18. The photoelectric conversion element according to claim 5,
wherein the photoelectric conversion film further contains an n-type semiconductor material and a p-type semiconductor material, and
the photoelectric conversion film has a bulk heterojunction structure formed in a state in which the first compound, the second compound, the n-type semiconductor material, and the p-type semiconductor material are mixed with each other.

19. The photoelectric conversion element according to claim 18,
wherein the n-type semiconductor material includes fullerenes selected from the group consisting of a fullerene and derivatives of the fullerene.

20. The photoelectric conversion element according to claim 5,
wherein the p-type semiconductor material has no absorption in a visible light region.
